(19) United States
(12) Patent Application Publication
(10) Pub. No.: US 2026/0274930 A1
(43) Pub. Date: Sep. 17, 2026

(54) ABETA-TARGETING PROTEINS AND METHODS OF USE

(71) Applicant: Denali Therapeutics Inc., South San Francisco, CA (US)

(72) Inventors: Kapil G. GADKAR, Redwood City, CA (US); Nathalie KHOURY, Sunnyvale, CA (US); Michelle E. PIZZO, Pacifica, CA (US); Joy Yu ZUCHERO, San Mateo, CA (US)

(21) Appl. No.: 19/168,034

(22) PCT Filed: Mar. 22, 2024
(86) PCT No.: PCT/US2024/021179

§ 371 Date: Sep. 23, 2025

Related U.S. Application Data
(60) Provisional application No. 63/492,124, filed on Mar. 24, 2023.

(51) Int. Cl. C07K 16/18; A61K 39/00; A61P 25/28; C07K 16/28

(52) U.S. Cl. CPC C07K 16/18; A61P 25/28; C07K 16/283; C07K 16/2881; A61K 2039/505; C07K 2317/31; C07K 2317/526; C07K 2317/55; C07K 2317/565; C07K 2317/71; C07K 2317/92; C07K 2319/00

(57) ABSTRACT

Described are Abeta-targeting proteins comprising an Abeta-binding region, a transferrin receptor (TfR)-binding region that specifically binds a TfR with an affinity of about 900 nM to about 10,000 nM, an FcγR-binding region. Also described are method of making the Abeta-targeting proteins and methods of using the Ab eta-targeting proteins to treat neurodegenerative disorders.

### RELATED APPLICATION

The application claims the benefit of priority to U.S. Provisional Application No. 63/492,124, filed on Mar. 24, 2023. The entire contents of the foregoing application are incorporated herein by reference.

### SEQUENCE LISTING

The Sequence Listing written in file 590729_SeqListing_ST26.xml is 34 kilobytes in size, was created Feb. 28, 2023, and is hereby incorporated by reference.

### BACKGROUND

Alzheimer's disease (AD) is a progressive neurodegenerative disorder clinically characterized by cognitive impairment, behavioral disturbances, psychiatric symptoms, and disability in activities of daily living. These clinical manifestations constitute AD dementia.

One of the hallmarks of the disease is the presence of amyloid beta plaques. It is hypothesized that the accumulation of Abeta plaques resulting from an imbalance between Abeta production and Abeta clearance in the brain contributes to pathogenesis.

Development of drugs to treat diseases such as AD, which require delivery to the brain, is complicated by the need to transport the drug across the blood brain barrier. While several potential disease-modifying drug candidates are currently under investigation, there are currently no therapies that reliably modify the course of Alzheimer's disease. Antibodies to Abeta, while offering the potential to provide some therapeutic benefit, suffer from side-effects, including edema (VE) and microhemorrhages (mH). Thus, there is a need in the art for improved therapeutics and methods for treating neurodegenerative disorders such as Alzheimer's disease.

### SUMMARY

Described are Abeta-targeting proteins comprising: (a) an Abeta-binding region, (b) a transferrin receptor (TfR)-binding region that specifically binds TfR with an affinity of about 900 nM to about 10,000 nM, and (c) an FcγR-binding region. The Abeta-targeting proteins are able to cross the blood brain barrier and bind to amyloid beta. The TfR-binding region facilitates transport of the Abeta-targeting protein across the blood brain barrier, thus facilitating delivery to the brain. Contrary to prediction, a TfR-binding region that specifically binds TfR with an affinity of about 900 nM to about 10,000 nM facilitates delivery of an amyloid beta-binding polypeptide to the brain at a higher initial rate compared to the initial rate of delivery of an amyloid beta-binding polypeptide linked to a TfR-binding polypeptide having a higher affinity for TfR. In some embodiments, the TfR-binding region specifically binds TfR with an affinity of about 900 nM to about 8500 nM, about 900 nM to about 6500 nM, about 900 nM to about 5000 nM, about 900 nM to about 3500 nM, about 900 nM to about 2500 nM, about 900 nM to about 1600 nM, or about 900 nM to about 1300 nM. In some embodiments, the TfR-binding region specifically binds TfR with an affinity of about 1000 nM to about 1200 nM. In some embodiments, the TfR-binding region specifically binds TfR with an affinity of about 1100 nM.

In some embodiments, the TfR-binding region specifically binds an apical domain of TfR. In some embodiments, the Abeta-targeting proteins are configured such that the TfR-binding region binds the TfR without inhibiting binding of transferrin to TfR. In some embodiments, the TfR is human TfR1.

TfR-binding can be provided by, e.g., a TfR-binding polypeptide, a TfR-binding polypeptide linked to an Fc polypeptide, a first Fc polypeptide comprising a CH3 domain modified to bind to TfR, an anti-TfR antibody or antigen-binding fragment thereof, or an anti-TfR antibody or antigen-binding fragment thereof linked to an Fc polypeptide. The Fc polypeptide can be derived from an IgG, e.g., a human IgG1, IgG2, IgG3, or IgG4. In some embodiments, TfR-binding is provided by an Fc polypeptide comprising a CH3 domain having amino acid substitutions at least six, seven, eight, nine, or ten of the positions selected from 380, 384, 386, 387, 388, 398, 390, 413, 415, 416, and 421 (e.g., N384Y, Q386T, P387E, E388W, N389V, D413T, S415E, R416E, and N421F), according to EU numbering scheme. In some embodiments, TfR-binding is provided by an Fc polypeptide comprising a CH3 domain having amino acid substitutions at eight, nine, or ten of the positions selected from 380, 384, 386, 387, 388, 389, 390, 413, 415, 416, and 421. In some embodiments, TfR-binding is provided by an Fc polypeptide comprising a CH3 domain having a tyrosine or phenylalanine at position 384, a threonine or asparagine at position 386, an aspartate at position 387, a tryptophan at position 388, a serine, threonine, or valine at position 389, a serine or asparagine at position 390, a threonine or serine at position 413, a serine or glutamine at position 415, a glutamate at position 416, and a phenylalanine or tyrosine at position 421, according to EU numbering scheme. An exemplary CH3 domain modified to specifically bind a TfR with an affinity of about 900 nM to about 10,000 nM (e.g., about 900 nM to about 2500 nM or about 900 nM to about 1300 nM) is provided by amino acids 348-453 or 348-454 of any of SEQ ID NOs:8, 10 and 17-31. In some embodiments, the TfR-binding region comprises a CH3 domain having any of the sets of substitutions listed in Table 1 and having an amino acid sequence having at least 85% identity, at least 90% identity, or at least 95% identity to amino acids 348-453 or 348-454 of any of SEQ ID NOs: 8, 10, and 17-31. In some embodiments, an Fc polypeptide having a TfR-binding region comprises a modified CH3 domain having at least 85% identity, at least 90% identity, or at least 95% identity to amino acids 348-454 of SEQ ID NO: 8 or 10 and having a tyrosine (Y) at position 384, a threonine (T) at position 386, a glutamate (E) at position 387, a tryptophan (W) at position 388, a valine (V) at position 389, a threonine (T) at position 413, a glutamate (E) at position 415, a glutamate (E) at position 416, and a phenylalanine (F) at position 421, according to EU numbering scheme. In some embodiments, an Fc polypeptide having a TfR-binding region comprises a modified CH3 domain having the amino acid sequence of amino acids 348-453 or 348-454 of any one of SEQ ID NOs: 8, 10, and 17-31. Where TfR binding is provided by an antigen-binding fragment of an anti-TfR antibody, the fragment can be, but is not limited to, a F(ab)2 fragment, a Fab fragment, or a single chain variable fragment (scFv).

FcγR-binding can be provided by an Fc polypeptide. The Fc polypeptide can be derived from an IgG, e.g., a human IgG1, IgG2, IgG3, or IgG4. In some embodiments, the Fc polypeptide providing FcγR-binding does not contain any modifications that reduce FcγR binding.

In some embodiments, an Abeta-targeting protein comprises an Fc dimer, wherein the dimer comprises a first Fc polypeptide and a second Fc polypeptide. In some embodiments, the Fc dimer is monovalent for TfR binding (i.e., either the first or the second Fc polypeptide, but not both, contain a TfR-binding region).

In some embodiments, the Abeta-targeting protein is configured to have reduced binding to an FcγR, when the Abeta-targeting protein is bound to TfR. Reduced binding to FcγR when the Abeta-targeting protein is bound to TfR can be accomplished by using an Fc dimer wherein a first Fc polypeptide of the Fc dimer contains a CH3 domain modified to bind to TfR and one or more amino acid substitutions that reduce binding to an FcγR, and wherein a second Fc polypeptide of the Fe dimer does not bind TfR and does not contain any modifications that reduce FcγR binding. Mutations that reduced FcγR binding include, but are not limited to L234A, L235A, and P329G substitutions, according to EU numbering scheme (e.g., L234A and L235A substitutions or L234A, L235A, and P329G substitutions).

Abeta-targeting proteins comprising an Fc dimer may have mutations in the Fc dimer polypeptides that promote heterodimerization. Such mutations include, but are not limited to, knob and hole mutations. A knob mutation can be a T366W substitution, according to EU numbering scheme. Hole mutations can be, but are not limited to T366S, T368A, and Y407V substitutions, according to EU numbering scheme. One Fc polypeptide of an Fc dimer can be modified to have a knob mutation, while the other Fc polypeptide of the Fc dime is modified to have hole mutations.

An exemplary Fc polypeptide having a TfR-binding region and one or more substitutions that reduce binding to an FcγR suitable for use in forming an Abeta-targeting protein is provided by amino acids 228-453 or 228-454 of any one of SEQ ID NOs:8, 10, and 17-31. In some embodiments, the Fc polypeptide having a TfR-binding region comprises a polypeptide having an amino acid sequence having at least 85% identity, at least 90% identity, or at least 95% identity to amino acids 228-453 or 228-454 of any of SEQ ID NOs: 8, 10, and 17-31 and having the amino acids at positions 384, 386, 387, 388, 389, 390, 413, 415, 416, and 421 (according to EU numbering scheme) as shown in Table 1. In some embodiments, an Fc polypeptide having a TfR-binding region comprises a polypeptide having at least 85% identity, at least 90% identity, or at least 95% identity to amino acids 228-454 of SEQ ID NO:8 or 10 and having an alanine at position 234, an alanine at position 235, optionally a glycine at position 329, optionally a tryptophan at position 366, a tyrosine at position 384, a threonine at position 386, a glutamate at position 387, a tryptophan at position 388, a valine at position 389, a threonine at position 413, a glutamate at position 415, a glutamate at position 416, and a phenylalanine at position 421, according to EU numbering scheme. In some embodiments, an Fc polypeptide having a TfR-binding region comprises a polypeptide having the amino acid sequence of amino acids 228-453 or 228-454 of any of SEQ ID NOs:8, 10, and 17-31.

An exemplary Fc polypeptide having an FcγR-binding region suitable for use in forming an Abeta-targeting protein is provided by amino acids 228-454 of SEQ ID NO:9. In some embodiments, an Fc polypeptide having an FcγR-binding region comprises a polypeptide having at least 85% identity, at least 90% identity, or at least 95% identity to amino acids 228-454 of SEQ ID NO:9, wherein the polypeptide optionally has a serine at position 366, an alanine at position 368 and a valine at position 407, according to EU numbering scheme. In some embodiments, an Fc polypeptide having an FcγR-binding region comprises a polypeptide having the amino acid sequence of amino acids 228-453 or 228-454 of SEQ ID NO:9.

The Abeta-binding region of an Abeta-targeting protein can be, but is not limited to: an anti-Abeta antibody of an antigen-binding region thereof. In some embodiments, the anti-Abeta Fab binds to amyloid beta plaques and fibrils. The anti-Abeta antibody of an antigen-binding region thereof can be derived from an anti-Abeta antibody known in the art. In some embodiments, the Abeta-binding region of an Abeta-targeting protein comprises the CDR sequences of an anti-Abeta antibody known in the art. An antigen-binding region can be, but is not limited to, a Fab (or F(ab)2) or an scFv. In some embodiments, the Abeta-binding region comprises an anti-Abeta Fab. In some embodiments, an anti-Abeta Fab is linked to an Fc polypeptide having a TfR-binding region or an FcγR-binding region. In some embodiments, an Abeta-targeting protein comprises a first anti-Abeta Fab linked to a first Fc polypeptide having a TfR-binding region and a second anti-Abeta Fab linked to a second Fc polypeptide having an FcγR-binding region, wherein the first and second Fc polypeptide form an Fc dimer.

In some embodiments, an Abeta-targeting protein comprises two antibody light chains, a first antibody heavy chain comprising an Fc polypeptide having a CH3 domain modified to have a TfR-binding region and optionally one or more mutations that reduce FcγR binding, and a second antibody heavy chain comprising a Fc polypeptide having an FcγR-binding region.

In some embodiments, an Abeta-targeting protein binds Abeta and mediates effector function through the FcγR receptor binding region. In some embodiments, an Abeta-targeting protein binds amyloid plaques, cerebrovascular Abeta, or diffuse Abeta deposits and mediates effector function through the FcγR receptor binding region. In some embodiments, an Abeta-targeting protein is configured to have reduced FcγR receptor binding when the Abeta-targeting protein is bound to TfR. In some embodiments, the Abeta-targeting protein does not substantially deplete reticulocytes when administered to a subject in vivo.

Any of the described Abeta-targeting proteins can be provided in a pharmaceutically acceptable composition. The pharmaceutically acceptable composition can comprise one or more pharmaceutically actable excipients. The pharmaceutical composition can be formulated for use in combination with an additional agent useful for treating a neurodegenerative disease, e.g., Alzheimer's disease.

Nucleic acids encoding the Abeta-targeting proteins are provided. In some embodiments, a nucleic acid encoding an Abeta-targeting protein antibody heavy chain encodes a polypeptide having at least 85% identity, at least 90% identity, or at least 95% identity, to any of SEQ ID NOs:8, 10, and 17-31 wherein the encoded polypeptide has the amino acids at positions 384, 386, 387, 388, 389, 390, 413, 415, 416, and 421 (according to EU numbering scheme) as shown in Table 1. In some embodiments, a nucleic acid encoding an Abeta-targeting protein antibody heavy chain encodes a polypeptide having at least 85% identity, at least 90% identity, or at least 95% identity, to SEQ ID NO:8 or 10, wherein the encoded polypeptide contains: (a) an alanine at position 234, an alanine at position 235, a glycine at position 329, a tryptophan at position 366, a tyrosine at position 384, a threonine at position 386, a glutamate position 387, a tryptophan at position 388, a valine at position 389, a threonine at position 413, a glutamate at position 415, a glutamate at position 416, and a phenylalanine at position 421; or (b) an alanine at position 234, an alanine at position 235, a tryptophan at position 366, a tyrosine at position 384, a threonine at position 386, a glutamate position 387, a tryptophan at position 388, a valine at position 389, a threonine at position 413, a glutamate at position 415, a glutamate at position 416, and a phenylalanine at position 421. In some embodiments, a nucleic acid encoding an Abeta-targeting protein antibody heavy chain encodes a polypeptide having the amino acid sequence of any of SEQ ID NOs:8, 10, and 17-31.

Also described are cells comprising one or more nucleic acids encoding all or a portion of an Abeta-targeting protein. In some embodiments, the cells comprise: (a) a first nucleic acid encoding a polypeptide having at least 85% identity, at least 90% identity, at least 95% identity, or 100% identify to any of SEQ ID NOs:8, 10, and 17-31; (b) a second nucleic acid encoding a polypeptide having at least 85% identity, at least 90% identity, at least 95% identity, or 100% identify to SEQ ID NO:9; and (c) a third nucleic acid encoding a polypeptide having at least 85% identity, at least 90% identity, at least 95% identity, or 100% identify to SEQ ID NO:7.

Described are methods of producing an Abeta-targeting protein comprising: culturing a host cell comprising one or more nucleic acids encoding an Abeta-targeting protein under conditions suitable for expression of the nucleic acids, and isolating the Abeta-targeting protein from the culture.

Described are methods of reducing amyloid plaques in a brain of a subject, the methods comprising administering to the subject an Abeta-targeting protein. The Abeta-targeting proteins can be administered to a subject that has cognitive impairment, memory loss, dementia, or loss of neuronal connections in the brain or has or is at risk of developing a disease associated with accumulation of Abeta or amyloid plaques. In some embodiments, the subject has or has been diagnosed with Alzheimer's disease or is at increased risk of developing Alzheimer's disease.

Described are methods of treating a neurodegenerative disease in a subject, comprising administering to the subject an Abeta-targeting protein. In some embodiments, the neurodegenerative disease is Alzheimer's disease. In some embodiments, the method further comprises administering to the subject at least one additional agent useful for treating Alzheimer's disease.

Described are methods of increasing Abeta phagocytosis and/or increasing recruitment of microglia to Abeta-positive plaques in a subject comprising administering to the subject an Abeta-targeting protein.

Described are methods of reducing amyloid-related imaging abnormalities (ARIAs) in a subject comprising administering to the subject an Abeta-targeting protein.

### BRIEF DESCRIPTION OF THE DRAWINGS

FIG. 1. Graphs illustrating binding of ATVs to oligomeric Abeta₁₋₄₂ and fibrillar Abeta₁₋₄₂, and monomeric Abeta₁₋₄₀.

FIG. 2A-C. Graph illustrating (A) plasma clearance and (B) brain pharmacokinetics (PK) of injected doses of Abeta targeting protein ATV35.23.3^{cisLALA}:Abeta and ATV35.23.4^{cisLALA}:Abeta, and (C) reticulocytes following administration of Abeta targeting protein ATV35.23.3^{cisLALA}:Abeta and ATV35.23.4:Abeta in TfR^{mu/hu} mice.

FIG. 2D-G. Graphs illustrating (D) human IgG concentration in plasma, (E) human IgG concentration in brain, (F) blood reticulocyte levels, or (G) bone marrow reticulocyte levels following injections of anti-Abeta IgG antibodies, ATV35.23.3:Abeta, ATV35.23.3^{cisLALA}:Abeta, or ATV35.23.3^{LALA}:Abeta in TfR^{mu/hu} mice.

FIG. 3. (A) Diagrams of embodiments of Abeta-targeting proteins. (B) Graph illustrating brain IgG concentration of the Abeta-targeting proteins shown in A. (C) Graph illustrating effect on blood reticulocytes following administration Abeta-targeting proteins shown in A in TfR^{mu/hu} mice.

FIG. 4. Graphs illustrating (A) plasma huIgG levels, (B) brain huIgG levels, (C) huIgG intensity in plaques, (D) percent plaque that are colocalized with CD68, and (E) percent area of plaques following administration of Anti-Abeta IgG, ATV: 35.23.4^{cisLALA}:Abeta, and ATV35.23.3^{cisLALA}:Abeta in App^{SAA} KI TfR^{mu/hu} mice.

FIG. 5. Graph illustrating plasma PK following administration of the indicated ATV:Abeta molecules in wild type mice.

FIG. 6A. Graph illustrating dynamic light scattering analysis of fluorescein (FAM)-labeled amyloid beta 1-42 fibrils.

FIG. 6B. Diagram illustrating FACS analysis of FAM-labelled amyloid beta fibrils in TfR^{mu/hu} mice following administration of ATV:Abeta molecules.

FIG. 6C-D. Graphs illustrating (C) Abeta positive microglia and (D) signal intensity of Abeta in microglia following injection of the indicated ATV:Abeta molecules in TfR^{mu/hu} mice.

FIG. 7. Graphs illustrating (A) immunodecoration analysis, (B) microglia recruitment to plaques, and (C) total plaque area following injection of the indicated ATV:Abeta molecules in APP^{SAA} KI; TfR^{mu/hu} KI mice.

FIG. 8A-B. Graphs illustrating levels of (A) blood reticulocytes and (B) hemoglobin in non-human primates following injection of the indicated ATV:Abeta molecules (figure legend shown in FIG. 8C).

FIG. 8C. Graph illustrating levels of red blood cells in non-human primates following administration of the indicated ATV:Abeta molecules.

FIG. 9. Graph illustrating plasma PK in non-human primates following administration of the indicated ATV:Abeta molecules.

FIG. 10A. Graph illustrating plasma PK profiles in the 5XFAD; TfR^{mu/hu} KI mice treated with ATV35.23.4^{cisLALA}, ATV35.23.4^{cisLALAPG}, or control IgG.

FIG. 10B-D. Graphs illustrating (B) brain concentrations of the ATV35.23.4^{cisLALA}, ATV35.23.4^{cisLALAPG}, or control IgG, (C) immunodecoration of plaques by ATV35.23.4^{cisLALA}, ATV35.23.4^{cisLALAPG}, or control IgG, and (D) recruitment of CD68 to 35-125 μm² plaques following administration of ATV35.23.4^{cisLALA}, ATV35.23.4^{cisLALAPG} or control IgG.

FIG. 11 depicts the number of 5xFAD: TfRmu/huKI mice with ARIA events following administration of anti-Abeta higG (10 mg/kg), anti-Abeta kLALA hIgG (10 mg/kg), ATV35.23.3:Abeta (3 mg/kg), and ATV35.23.3^{cisLALA}:Abeta (3 mg/kg).

FIG. 12 depicts differential route of entry into the brain for anti-Abeta vs. ATV:Abeta, due to preferential expression of TfR in capillaries and venules. As a result, ATV:Abeta displays a broader biodistribution into the brain parenchyma (cyan), as compared to anti-Abeta that remains primarily distributed into the brain vasculature one day after a single peripheral injection.

### DETAILED DESCRIPTION

### I. Definitions

As used herein, the singular forms “a,” “an,” and “the” include plural referents unless the content clearly dictates otherwise. Thus, for example, reference to “a polypeptide” may include two or more such molecules, and the like.

As used herein, the terms “about” and “approximately,” when used to modify an amount specified in a numeric value or range, indicates the numeric value as well as reasonable deviations from the value known to the skilled person in the art. In some embodiments, the term “about” means within the typical ranges of tolerances in the art. In some embodiments, the term “about” means within 1 or 2 standard deviations from the mean. In some embodiments, the term “about” means±10%. In some embodiments, the term “about” means±5%. When the term “about” is present before a series of numbers or a range, it is understood that “about” can modify each of the numbers in the series or range.

A “transferrin receptor” or “TfR” as used in the context of this invention refers to transferrin receptor protein 1. The human transferrin receptor 1 polypeptide sequence is set forth in SEQ ID NO:15. Transferrin receptor protein 1 sequences from other species are also known (e.g., chimpanzee, accession number XP 003310238.1; rhesus monkey, NP_001244232.1; dog, NP_001003111.1; cattle, NP_001193506.1; mouse, NP 035768.1; rat, P_073203.1; and chicken, P_990587.1). The term “transferrin receptor” also encompasses allelic variants of exemplary reference sequences, e.g., human sequences, that are encoded by a gene at a transferrin receptor protein 1 chromosomal locus. Full length transferrin receptor protein includes a short N-terminal intracellular region, a transmembrane region, and a large extracellular domain. The extracellular domain is characterized by three domains: a protease-like domain, a helical domain, and an apical domain. The apical domain sequence of human transferrin receptor 1 is set forth in SEQ ID NO:4.

An “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region. In general, an Fc polypeptide does not contain a variable region.

An “modified Fc polypeptide” is an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

An “Fc polypeptide dimer” refers to a dimer of two Fc polypeptides. In some embodiments, an Fc polypeptide dimer is capable of binding an Fc receptor (e.g., FcγR). In an Fc polypeptide dimer, the two Fc polypeptides dimerize by the interaction between the two CH3 antibody constant domains. In some embodiments, the two Fc polypeptides may also dimerize via one or more disulfide bonds that form between the hinge domains of the two dimerizing Fc domain monomers. An Fc polypeptide dimer can be a heterodimer or a homodimer. An Fc polypeptide dimer may comprise two wild-type Fc polypeptides, a wild-type Fc polypeptide and a modified Fc polypeptide, or two modified Fc polypeptides. For an Fc polypeptide dimer comprising two modified Fc polypeptide, the two modified Fc polypeptides may be the same or different.

The terms “CH3 domain” and “CH2 domain” as used herein refer to immunoglobulin constant region domain polypeptides. In the context of IgG antibodies, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme. CH2 and CH3 domain polypeptides may also be numbered by the EVIGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. In the context of IgG antibodies, an Fc region refers to the segment of amino acids from about position 231 to about position 447 as numbered according to the EU numbering scheme. As used herein, the term “Fc region” may also include at least a part of a hinge region of an antibody.

The terms “wild-type,” “native,” and “naturally occurring” with respect to a CH3 or CH2 domain are used herein to refer to a domain that has a sequence that occurs in nature.

The term “Fcγ receptor” or “FcγR” refers to one type of Fc receptors, which are classified based on the type of antibody that they recognized. FcγRs includes several members, FcγRI (CD64), FcγRIIA (CD32), FcγRIIB (CD32), FcγRIIIA (CD16a), and FcγRIIIB (CD16b), which differ in their antibody affinities due to different molecular structures. FcγRs bind to the Fc portion of IgG class of antibodies and are crucial for inducing phagocytosis of opsonized microbes. FcγRs are found on the cell surface of cells in the immune system. FcγRs are responsible for eliciting immune system effector functions and are activated upon binding of the Fc portion of an antibody to the receptor. FcγRs mediate immune functions, e.g., binding to antibodies that are attached to infected cells or invading pathogens, stimulating phagocytic or cytotoxic cells to destroy microbes or infected cells by antibody-mediated phagocytosis or ADCC.

The term “variable region” refers to a domain in an antibody heavy chain or light chain derived from a germline Variable (V) gene, Diversity (D) (heavy chain only) gene, and Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions” (CDRs).

A variant, with respect to a given sequence, is an alteration in a nucleic acid or amino acid sequence relative to a reference (e.g., wild-type or most common) sequence. A variant can be a naturally occurring sequence (e.g., an allelic variant) and non-naturally occurring sequence. A non-naturally occurring variant domain refers to a variant or mutant domain that is not present in a cell in nature and that is produced by genetic modification (e.g., using genetic engineering technology or mutagenesis techniques) of a native domain. An alteration in a nucleic acid or amino acid sequence (e.g., a mutation) can include one or more substitutions, one or more insertions, one or more deletions, or combinations thereof.

The term “conservative substitution,” “conservative mutation,” or “conservatively modified variant” refers to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gin (Glutamine or Q), Lys (Lysine or K), Arg (Arginine or R), and His (Histidine or H); an “aromatic group” including Phe (Phenylalanine or F), Tyr (Tyrosine or Y), Tip (Tryptophan or W), and (Histidine or H); and an “aliphatic group” including Gly (Glycine or G), Ala (Alanine or A), Val (Valine or V), Leu (Leucine or L), He (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged subgroup” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acids, e.g., in the active site, to preserve hydrophobicity.

“Binding affinity” refers to the strength of the non-covalent interaction between two molecules, e.g., between single binding region or site on a protein and a target. Binding affinity may be quantified by measuring an equilibrium dissociation constant (KD), which refers to the dissociation rate constant (k_{d}, time⁻¹) divided by the association rate constant (kₐ, time⁻¹M⁻¹). KD can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet® platform). The term “binding affinity” includes not only formal binding affinities, such as those reflecting 1:1 interactions between a polypeptide and its target, but also apparent affinities for which KD's are calculated that may reflect avid binding (avidity).

The term “specifically binds” or “selectively binds” to a target (e.g., amyloid beta, TfR, or FcγR) when referring to a binding region (e.g., Abeta-binding region, TfR-binding region, or FcγR-binding region) refers to a binding reaction whereby the binding region binds to the target with greater affinity, greater avidity, and/or greater duration than it binds to a structurally different target. A binding region may have at least 5-fold, 6-fold, 7-fold, 8-fold, 9-fold, 10-fold, 20-fold, 25-fold, 50-fold, 100-fold, 1,000-fold, 10,000-fold, or greater affinity for a specific target compared to an unrelated target when assayed under the same affinity assay conditions. The term “specific binding,” “specifically binds to,” or “is specific for” a particular target can be exhibited, for example, by a molecule having an equilibrium dissociation constant KD for the target to which it binds of, e.g., 10⁻⁴ M or smaller (e.g., 10⁻⁵ M, 10⁻⁶ M (1000 nM), 10⁻⁷M (100 nM), 10⁻⁸ M (10 nM), 10⁻⁹ M (1 nM), 10⁻¹⁰ M, 10⁻¹¹ M, or 10⁻¹² M). In some embodiments, an binding region specifically binds to an target (e.g., protein) that is conserved among species, (e.g., structurally conserved among species).

The term “amino acid” refers to naturally occurring and synthetic amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, γ-carboxyglutamate and O-phosphoserine. “Amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid.

Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (He), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof. Amino acids may be referred to herein by either their commonly known three letter symbols or by the one-letter symbols recommended by the IUPAC-IUB Biochemical Nomenclature Commission.

As used herein, the term “mutant” with respect to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions. Substitutions of a single amino acid can be indicated by a single letter amino acid symbol indicating the amino acid prior to the substitution, followed by a number indicating the amino acid position, followed by a one letter amino acid symbol indicating the amino acid substitution (e.g., T366W indicates a threonine at position 366 is modified to be a tryptophan). Substitutions of a single amino acid can also be indicated by a number indicating the amino acid position at which this is a substitution followed by a one letter amino acid symbol indicating the amino acid substitution (e.g., 366W indicates a tryptophan at position).

A “polypeptide” is a polymer of two or more amino acid residues in a single chain. The term applies to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide, a polypeptide dimer, or polypeptide multimer. A polypeptide dimer can be a homodimer or a heterodimer. A polypeptide multimer can be a homomultimer or a heteromultimer. A heteromultimer may comprise two or more copies of any give single chain polypeptide. For example, an immunoglobulin is a heteromultimer comprises two heavy chains and two light chains. The tow heavy chains can be the same or different the two light chains can be the same or different. The single polypeptide chains of a dimer or multimer may be joined by one or more covalent bonds (e.g., a disulfide bonds), by non-covalent interactions, or by a combination thereof.

The terms “identical” or percent “identity,” in the context of two or more nucleic acid or polypeptide sequences, refer to two or more sequences or subsequences that are the same or have a specified percentage of nucleic acid or amino acid residues (e.g., at least 60%, at least 65%, at least 70%), at least 75%, at least 80%, at least 85%, at least 90%, or at least 95% or greater) that are identical over a specified region when compared and aligned for maximum correspondence over a comparison window or designated region, as measured using a sequence comparison algorithm or by manual alignment and visual inspection. For sequence comparison, typically one sequence acts as a reference sequence to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given nucleotide or amino acid residue in a nucleic acid or polypeptide sequence, refers to the position of the residue of a specified reference sequence when the given amino acid sequence is maximally aligned and compared to the reference sequence. The sequence that is aligned to the reference sequence need not be the same length as the reference sequence.

The “EU numbering scheme” is generally used in the art when referring to a residue in an antibody heavy chain constant region. The EU numbering scheme is shown below with respect to SEQ ID NO:8:

```
QVQLVES
GGGVVQPGRSLRLSCAASGFAFSSYGMHWVRQAPGKGLEWVAVIWEDGTK
KYYTDSVKGRFTISRDNSKNTLYLQMNTLRAEDTAVYYCARDRGIGARRG
EU 120 130 140 150
                                      | | | |
PYYMDVWGKGTTVTVSSASTKGPSVFPLAPSSKSTSGGTAALGCLVKDYF
EU 160 170 180 190 200
                  | | | | |
PEPVTVSWNSGALTSGVHTFPAVLQSSGLYSLSSVVTVPSSSLGTQTYIC
EU 210 220 230 240 250
                  | | | | |
NVNHKPSNTKVDKKVEPKSCDKTHTCPPCPAPEAAGGPSVFLFPPKPKDT
EU 260 270 280 290 300
                  | | | | |
LMISRTPEVTCVVVDVSHEDPEVKFNWYVDGVEVHNAKTKPREEQYNSTY
EU 310 320 330 340 350
                  | | | | |
RVVSVLTVLHQDWLNGKEYKCKVSNKALGAPIEKTISKAKGQPREPQVYT
EU 360 370 380 390 400
                  | | | | |
LPPSRDELTKNQVSLWCLVKGFYPSDIAVEWESYGTEWVNYKTTPPVLDS
EU 410 420 430 440
                  | | | |
DGSFFLYSKLTVTKEEWQQGFVFSCSVMHEALHNHYTQKSLSLSPGK.
```

The term “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In some embodiments, the subject is a human.

The terms “treat,” “treatment,” and the like, mean the methods or steps taken to provide relief from, or amelioration or alleviation of the number, severity, adverse effect, and/or frequency of one or more symptoms or pathological consequences of a disease, disorder, or condition in a subject. Treatment can be prophylactic in terms of preventing or partially preventing a disease, or a symptom or condition of the disease. Preventing includes providing prophylaxis with respect to the occurrence or recurrence of a disease in a subject that may be predisposed to the disease but has not yet been diagnosed with the disease. Preventing also includes providing prophylaxis with respect to the occurrence or recurrence of a symptom or pathological consequence of a disease in a subject that may be predisposed symptom or pathological consequence of the disease but has not yet been diagnosed with the symptom or pathological consequence the disease. Treatment can also be prophylactic in terms of delaying onset of a disease, or a symptom or condition of the disease. Delaying development of a disease or symptom or pathological consequence of the disease indicates deferring, hindering, slowing, retarding, stabilizing, suppressing, and/or postponing development of the disease or symptom or pathological consequence of the disease. The delay can be of varying lengths of time, depending on the history of the disease and/or individual being treated. Treating can include inhibiting the disease, disorder or condition, e.g., impeding its progress; and relieving the disease, disorder, or condition, e.g., causing regression of the disease, disorder and/or condition. Treatment can also mean prolonging survival as compared to expected survival in the absence of treatment. Treatment can be therapeutic in terms of a partial or complete cure of a disease, condition, symptom or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those previously diagnosed with a disease, disorder, or condition, or those identified as being at risk of developing a disease, disorder, or condition. Treating the disease, disorder, or condition can include ameliorating at least one symptom of the particular disease, disorder, or condition, even if the underlying pathophysiology is not affected.

The term “pharmaceutically acceptable excipient” refers to a non-active pharmaceutical ingredient that is biologically or pharmacologically compatible for use in humans or animals, such as but not limited to a buffer, carrier, or preservative.

An “effective amount” of an agent, e.g., an Abeta-targeting protein or a pharmaceutical formulation containing an Abeta-targeting protein, in the context of administration, refers to an amount effective, at dosages/amounts and for periods of time necessary, to achieve a desired result, such as a therapeutic or prophylactic result.

A “therapeutically effective amount” of an agent, e.g., an Abeta-targeting protein or a pharmaceutical formulation containing an Abeta-targeting protein, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

A “dose,” “unit dose,” or “dosage” refers to physically discrete units suitable for use in a subject, each unit containing a predetermined quantity of active pharmaceutical ingredient and/or a pharmaceutical composition

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, the compositions described herein are administered intravenously.

### II. ABETA-Targeting Proteins

Linkage of a transferrin receptor binding region of a molecule can be used to increase transport of the molecule across the blood brain barrier (BBB). For some molecules, such as antisense oligonucleotides, rapid delivery to the brain is preferred. From other compounds, initial rate of delivery is less important than persistence. TfR binding polypeptides with higher affinities for TfR typically (e.g., less than 900 nM binding affinity) result in higher initial rates of delivery to the brain followed by rapid clearance. In contrast, TfR binding polypeptides with low affinities for TfR typically have lower initial rates of delivery to the brain, but increased brain persistence compared to higher affinity TfR binding polypeptides. Surprisingly, linkage of a TfR binding polypeptide having affinity for TfR of about 900 nM to about 10,000 nM (e.g., about 900 nM to about 2500 nM or about 900 nM to about 1300 nM) to an amyloid beta-binding polypeptide results in an initial rate of delivery (Cmax) of the amyloid beta-binding polypeptide to the brain that is higher compared to the initial rate of delivery of the amyloid beta-binding polypeptide linked to a TfR-binding polypeptide having a higher affinity for Tfr.

Described are Abeta-targeting proteins and compositions capable of crossing the blood brain barrier and binding to amyloid beta plaques. The Abeta-targeting proteins comprise a Abeta-binding region, a TfR-binding region that specifically binds a TfR with an affinity of about 900 nM to about 10,000 nM (e.g., about 900 nM to about 2500 nM or about 900 nM to about 1300 nM), and an FcγR-binding region. The described Abeta-targeting proteins are transported across the blood brain barrier (BBB). Because the Abeta-targeting proteins possess Abeta binding properties and immune effector function properties, Abeta-targeting proteins can be administered to a subject to treat amyloid plaques. Contrary to expectation, use of a TfR-binding region that specifically binds a TfR with an affinity of about 900 nM to about 10,000 nM (e.g., about 900 nM to about 2500 nM or about 900 nM to about 1300 nM), when linked to an Abeta-binding polypeptide, results in increased delivery of the proteins to the brain, and to a greater extent than a TfR-binding region having higher TfR affinity. Further, when linked to an Abeta-binding polypeptide, TFR-binding regions that specifically bind TfR with an affinity of about 900 nM to about 10,000 nM (e.g., about 900 nM to about 2500 nM or about 900 nM to about 1300 nM also exhibit increased brain retention of the Abeta-targeting protein. The combination of higher initial rate of brain delivery and higher brain retention is useful for therapies designed to target amyloid beta plaques and fibrils.

Abeta-targeting proteins, compositions containing the Abeta-targeting proteins, nucleic acids encoding the Abeta-targeting proteins, a methods of manufacturing Abeta-targeting proteins are described.

Methods of using the Abeta-targeting proteins to treat diseases or conditions mediated by amyloid beta, including neurodegenerative diseases such as Alzheimer's disease.

### A. Amyloid Beta (Abeta)-Binding Region

Amyloid beta (Abeta) is a peptide, 36-43 amino acids in length, that is the main component of the amyloid plaques (extracellular deposits) found in the brains of people with Alzheimer's disease. The plaques are composed of a tangle of Abeta oligomers and regularly ordered aggregates called amyloid fibrils. The soluble oligomeric forms of Abeta may be causative agents in the development of Alzheimer's disease.

An Abeta-binding region comprises a polypeptide or a region or domain or a polypeptide or protein that specifically binds Abeta. The Abeta-binding region can specifically bind to oligomeric Abeta and/or fibrillar Abeta. In some embodiments, the Abeta-binding region binds to oligomeric Abeta. In some embodiments, the Abeta-binding region binds to fibrillar Abeta. In some embodiments, the Abeta-binding region binds to both oligomeric Abeta and fibrillar Abeta.

The Abeta-targeting protein can be monovalent or multivalent for the Abeta-binding. In other words, the Abeta-targeting protein can contain a single Abeta-binding region or the Abeta-targeting protein can contain multiple Abeta-binding regions. In some embodiments, the Abeta-targeting protein comprises two or more Abeta-binding regions. The two or more Abeta-binding regions can be the same or different. In some embodiments, the Abeta-targeting protein comprises two Abeta-binding regions.

An Abeta-binding region can be, but is not limited to, a peptide, an engineered peptide, an anti-Abeta antibody, or a Abeta-binding fragment of an anti-Abeta antibody.

Anti-Abeta antibodies are known in the art and are available from various commercial sources. An anti-Abeta can be an antibody to an Abeta from a species other than human provided the antibody binds human Abeta. In some embodiments, the anti-Abeta antibody or the Abeta-binding fragment of an anti-Abeta antibody binds to oligomeric Abeta and fibrillar Abeta.

An Abeta-binding fragment of an anti-Abeta antibody can be, but is not limited to, a F(ab)2 fragment, a Fab fragment, or a single chain variable fragment (scFv).

In some embodiments, Abeta-binding region comprises an antigen-binding domain of an anti-Abeta antibody. In some embodiments, the Abet-binding region comprises a Fab region of an anti-Abeta antibody, two Fab regions of an anti-Abeta antibody, a F(ab)2 region of an anti-Abeta antibody, or at least one scFv derived from an anti-Abeta antibody. In some embodiments, the Abeta-binding region comprises an antigen-binding domain of aducanumab. In some embodiments, the Abeta-binding region comprises a Fab region of an aducanumab, two Fab regions of aducanumab, a F(ab)2 region of aducanumab, or at least one scFv derived from aducanumab.

In some embodiments, an Abeta-binding region comprises a heavy chain variable region (VH) and a light chain variable region (VL), wherein
(a) the VH comprises a first heavy chain complementarity determining region (VHCDR1) having the amino acid sequence of SEQ ID NO:1, a second heavy chain complementarity determining region (VHCDR2) having the amino acid sequence of SEQ ID NO: 2, and a third heavy chain complementarity determining region (VHCDR3) having the amino acid sequence of SEQ ID NO:3; and
(b) the VL comprises a first light chain complementarity determining region (VLCDR1) having the amino acid sequence of SEQ ID NO:4, a second light chain complementarity determining region (VLCDR2) having the amino acid sequence of SEQ ID NO: 5, and a third light chain complementarity determining region (VLCDR3) having the amino acid sequence of SEQ ID NO:6.
   The VH and VL can be part of a Fab, a F(ab)2, or an scFv.

In some embodiments, an Abeta-binding region comprises a VH and a VL, wherein
(a) the VH comprises an amino acid sequence having at least 85% identity, at least 90% identity, or at least 95% identity to amino acids 1-120 of SEQ ID NO:8 or 10 and wherein the VH comprises a VHCDR1 having the amino acid sequence of SEQ ID NO:1, a VHCDR2 having the amino acid sequence of SEQ ID NO:2, and a VHCDR3 having the amino acid sequence of SEQ ID NO:3; and
(b) the VL comprises an amino acid sequence having at least 85% identity, at least 90% identity, or at least 95% identity to amino acids 1-109 of SEQ ID NO:7 and wherein the VL comprises a VLCDR1 having the amino acid sequence of SEQ ID NO:4, a VLCDR2 having the amino acid sequence of SEQ ID NO:5, and a VLCDR3 having the amino acid sequence of SEQ ID NO:6.
   The VH and VL can be part of a Fab, a F(ab)2, or an scFv.

In some embodiments, an Abeta-binding region comprises a VH and a VL, wherein the VH comprises the amino acid sequence of amino acids 1-120 of SEQ ID NO:8 or 10 and the VL the amino acid of amino acids 1-109 of SEQ ID NO:7. The VH and VL can be part of a Fab, a F(ab)2, or an scFv.

In some embodiments, an Abeta-binding region comprises a heavy chain Fab region and a light chain Fab region, wherein
(a) the heavy chain Fab region comprises an amino acid sequence having at least 85% identity, at least 90% identity, or at least 95% identity to amino acids 1-227 of SEQ ID NO: 8 or 10 and comprises a VHCDR1 having the amino acid sequence of SEQ ID NO:1, a VHCDR2 having the amino acid sequence of SEQ ID NO:2, and a VHCDR3 having the amino acid sequence of SEQ ID NO:3, and
(b) the light chain Fab region comprises an amino acid sequence having at least 85% identity, at least 90% identity, or at least 95% identity to SEQ ID NO:7 and comprises a VLCDR1 having the amino acid sequence of SEQ ID NO:4, a VLCDR2 having the amino acid sequence of SEQ ID NO:5, and a VLCDR3 having the amino acid sequence of SEQ ID NO:6.

In some embodiments, an Abeta-binding region comprises a F(ab)2 region having two heavy chain Fab regions and two light chain Fab regions, wherein
(a) each heavy chain Fab region comprises an amino acid sequence having at least 85% identity, at least 90% identity, or at least 95% identity to amino acids 1-227 of SEQ ID NO: 8 or 10 and comprises a VHCDR1 having the amino acid sequence of SEQ ID NO:1, a VHCDR2 having the amino acid sequence of SEQ ID NO:2, and a VHCDR3 having the amino acid sequence of SEQ ID NO:3, and
(b) each light chain Fab region comprises an amino acid sequence having at least 85% identity, at least 90% identity, or at least 95% identity to SEQ ID NO:7 and comprises a VLCDR1 having the amino acid sequence of SEQ ID NO:4, a VLCDR2 having the amino acid sequence of SEQ ID NO:5, and a VLCDR3 having the amino acid sequence of SEQ ID NO:6.

In some embodiments, an Abeta-binding region comprises a heavy chain Fab region and a light chain Fab region, wherein the heavy chain Fab region comprises the amino acid sequence of amino acids of 1-227 of SEQ ID NO:8 or 10 and the light chain Fab region comprises the amino acid sequence of SEQ ID NO:7.

In some embodiments, an Abeta-binding region comprises a F(ab)2 region having two heavy chain Fab regions and two light chain Fab regions, wherein each heavy chain Fab region comprises the amino acid sequence of amino acids of 1-227 of SEQ ID NO:8 or 10 and each light chain Fab region comprises the amino acid sequence of SEQ ID NO:7.

### B. Transferrin Receptor (TfR)-Binding Region

A TfR-binding region is a molecule such as a polypeptide or a region or domain of a larger polypeptide or protein that specifically binds to a TfR, such as a human TfR. The TfR-binding region of the Abeta-targeting protein specifically binds to a TfR and facilitates transport of the Abeta-targeting protein across the blood brain barrier (e.g., by receptor-mediated transcytosis) or increase increases penetration of the Abeta-targeting protein into the brain.

The transferrin receptor (TfR) (also termed cluster of differentiation 71 (CD71)) binds transferrin (Tf) and performs a critical role in cellular iron uptake through interaction with iron-bound transferrin. TfR is highly expressed by brain capillary endothelial cells (BCECs) forming the blood-brain barrier (BBB). TfR is a 90 kDa type II transmembrane glycoprotein consisting of 760 amino acids that is found as a dimer (180 kDa) linked by disulfide bonds on the cell surface. The TfR1 monomer is composed of a large extracellular, C-terminal domain of 671 amino acids containing the Tf binding site, a transmembrane domain (28 amino acids), and an intracellular N-terminal domain (61 amino acids). The C-terminal extracellular domain contains three N-linked glycosylation sites at asparagine residues 251, 317, and 727 and one O-linked glycosylation site at threonine 104, which are all required for adequate function of the receptor.

In some embodiments, the TfR-binding region binds to an apical domain of TfR. The apical domain comprises residues 189-383 of TfR. In some embodiments, the TfR-binding region binds at an epitope that comprises position 208 of the full length human transferrin receptor sequence. In some embodiments, a TfR-binding region binds to the apical domain of the TfR at an epitope that comprises positions 158, 188, 199, 207, 208, 209, 210, 211, 212, 213, 214, 215, and/or 294 of the full length human TfR sequence (SEQ ID NO:15).

In some embodiments, binding of the TfR-binding region to TfR does not inhibit binding of transferrin to TfR. In some embodiments, binding of the Abeta-targeting protein to TfR does not inhibit binding of transferrin to TfR. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%).

In some embodiments, the TfR-binding region specifically binds a human TfR with an affinity of about 900 nM to about 10,000 nM. In some embodiments, the TfR-binding region specifically binds a human TfR with an affinity of about 900 nM to about 8500 nM. In some embodiments, the TfR-binding region specifically binds a human TfR with an affinity of about 900 nM to about 6500 nM. In some embodiments, the TfR-binding region specifically binds a human TfR with an affinity of about 900 nM to about 5000 nM. In some embodiments, the TfR-binding region specifically binds a human TfR with an affinity of about 900 nM to about 3500 nM. In some embodiments, the TfR-binding region specifically binds a human TfR with an affinity of about 900 nM to about 2500 nM. In some embodiments, the TfR-binding region specifically binds a human TfR with an affinity of about 900 nM to about 2000 nM. In some embodiments, the TfR-binding region specifically binds a human TfR with an affinity of about 900 nM to about 1600 nM. In some embodiments, the TfR-binding region specifically binds a human TfR with an affinity of about 900 nM to about 1300 nM. In some embodiments, the TfR-binding region specifically binds a human TfR with an affinity of about 1000 nM to about 1200 nM. In some embodiments, the TfR-binding region specifically binds a human TfR with an affinity of about 1400 nM, about 1500 nM, about 1600 nM, about 1800 nM, about 2000 nM, about 2500 nM, about 3000 nM, about 3500 nM, about 4000 nM, about 4500 nM, about 5000 nM, about 5500 nM, about 6000 nM, about 6500 nM, about 7000 nM, about 7500 nM, about 8000 nM, about 8500 nM, about 9000 nM, or about 10,000 nM. In some embodiments, the TfR-binding region specifically binds a human TfR with an affinity of about 900 nM, about 950 nM, about 1000 nM, about 1050 nM, about 1100 nM, about 1150 nM, or about 1200 nM. In some embodiments, the TfR-binding region specifically binds a human TfR with an affinity of 1100±200 nM. In some embodiments, the TfR-binding region specifically binds a human TfR with an affinity of 1100±100 nM. In some embodiments, the TfR-binding region specifically binds a human TfR with an affinity of 1100±50 nM. In some embodiments, the TfR-binding region specifically binds a human TfR with an affinity of 1100 nM.

In some embodiments, Abeta-targeting protein specifically binds a human TfR with an affinity of about 900 nM to about 10,000 nM. In some embodiments, the TfR-binding region specifically binds a human TfR with an affinity of about 900 nM to about 8500 nM. In some embodiments, an Abeta-targeting protein specifically binds a human TfR with an affinity of about 900 nM to about 6500 nM. In some embodiments, an Abeta-targeting protein specifically binds a human TfR with an affinity of about 900 nM to about 5000 nM. In some embodiments, an Abeta-targeting protein specifically binds a human TfR with an affinity of about 900 nM to about 3500 nM. In some embodiments, an Abeta-targeting protein specifically binds a human TfR with an affinity of about 900 nM to about 2500 nM. In some embodiments, an Abeta-targeting protein specifically binds a human TfR with an affinity of about 900 nM to about 2000 nM. In some embodiments, an Abeta-targeting protein specifically binds a human TfR with an affinity of about 900 nM to about 1600 nM. In some embodiments, an Abeta-targeting protein specifically binds a human TfR with an affinity of about 900 nM to about 1300 nM. In some embodiments, an Abeta-targeting protein specifically binds a human TfR with an affinity of about 1400 nM, about 1500 nM, about 1600 nM, about 1800 nM, about 2000 nM, about 2500 nM, about 3000 nM, about 3500 nM, about 4000 nM, about 4500 nM, about 5000 nM, about 5500 nM, about 6000 nM, about 6500 nM, about 7000 nM, about 7500 nM, about 8000 nM, about 8500 nM, about 9000 nM, or about 10,000 nM. In some embodiments, the Abeta-targeting protein specifically binds a human TfR with an affinity of about 1000 nM to about 1200 nM. In some embodiments, the Abeta-targeting protein specifically binds a human TfR with an affinity of about 900 nM, about 950 nM, about 1000 nM, about 1050 nM, about 1100 nM, about 1150 nM, or about 1200 nM. In some embodiments, the Abeta-targeting protein specifically binds a human TfR with an affinity of 1100-200 nM. In some embodiments, the Abeta-targeting protein specifically binds a human TfR with an affinity of 1100±100 nM. In some embodiments, the Abeta-targeting protein specifically binds a human TfR with an affinity of 1100±50 nM. In some embodiments, the Abeta-targeting protein specifically binds a human TfR with an affinity of 1100 nM.

Analyzing binding affinity, binding kinetics, and cross-reactivity between a TfR-binding region and a TfR can be done using methods available in the art for analyzing binding affinity, binding kinetics, and cross-reactivity between two polypeptides. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExAR), flow cytometry, fluorescence-activated cell sorting (FACS), BioLayer interferometry (e.g., Octet® (ForteBio, Inc., Menlo Park, CA)), and Western blot analysis. In some embodiments, ELISA is used to determine binding affinity and/or cross reactivity. Methods for performing ELISA assays are known in the art. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity. In some embodiments, binding affinity is determined by surface plasmon resonance (optionally using a Biacore™ instrument). In this method, a TfR-binding polypeptide is captured on a sensor chip and serial dilutions of TfR are injected onto the sensor chip at a specified flow rate (e.g., 30 μL/min) and temperature (e.g., room temperature). Samples are analyzed using specified association and dissociation times (e.g., 45 and 180 seconds, respectively), followed by sensor chip regeneration. Binding responses are corrected by subtracting the measured response from a control (e.g., using an irrelevant IgG at similar density) and then steady-state affinities can be determined by using software to fit the equilibrium response against concentration.

In some embodiments, the Abeta-targeting protein is monovalent for the TfR-binding region, meaning the Abeta-targeting protein contains a single TfR-binding region.

A TfR-binding region can be, but is not limited to, a peptide, an engineered peptide, an anti-TfR antibody, a TfR-binding fragment of an anti-TfR antibody, or a region of an antibody modified to bind to a human TfR.

Anti-TfR antibodies are known in the art and are available from various commercial sources. An anti-TfR can be an antibody to a TfR from a species other than human provided the antibody binds human TfR with an affinity as described above, e.g., about 900 nM to about 10,000 nM. In some embodiments, the anti-TfR antibody or the TfR-binding fragment of an anti-TfR antibody binds to an apical domain of the TfR. In some embodiments, binding of the anti-TfR antibody or the TfR-binding fragment of an anti-TfR antibody to the TfR does not inhibit binding of transferrin to the TfR. Exemplary anti-TfR antibodies include, but are not limited to, B3/25, RBC4, 7579, E2.3, A27.15, D65.30, D2C, ch128.1Av, ch128.1/IgG3, ch128.1/IgG1, hu128.1, (Candelaria et al. Front. Immunol. 12 (17 Mar. 2021), 2021), Ri7, 8D3 (Weber et al. Cell Reports 22:149-162, 2018). Exemplary anti-TfR antibodies are also described in U.S. patent publications: US2018282408A1, US2020071413A1, US20210138083, US20190092870, and US20130028891. An anti-TfR antibody can be modified to have an affinity for TfR of about 900 nM to about 10,000 nM.

A TfR-binding fragment of an anti-hTfR antibody can be, but is not limited to, a Fab fragment, or a single chain variable fragment (scFv), or a monovalent anti-TfR antibody, or a divalent antibody having a single TfR binding domain.

In some embodiments, TfR-binding region comprises an antigen-binding domain of an anti-TfR antibody. In some embodiments, the TfR-binding region comprises a Fab region of an anti-TfR antibody, or an scFv derived from an anti-TfR antibody.

A TfR-binding region can be derived from a protein known to bind the TfR, such as, P. vivax reticulocyte-binding protein 2b (PvRBP2b) or a viral protein such as an arenavirus protein (e.g., Machupo, Sabiá, Junín, Guanarito or Chapare virus) known to bind TfR.

In some embodiments, the TfR-binding region comprises an engineered polypeptide. An engineered polypeptide can be a polypeptide (e.g., an antibody Fc polypeptide) or antigen-binding region of an anti-TfR antibody modified to alter affinity of the polypeptide or the antigen-binding region of the anti-TfR antibody for the Tfr. Engineered peptides can be identified or generated using methods available in the art for identifying or generating a peptide having affinity for a known target (e.g., TfR). Such methods include, but are not limited to, phage display, yeast display (e.g., yest surface display), and directed evolution, and combinations thereof.

Methods of engineering a CH2 or CH3 domain to have transferrin receptor binding have been previously described in US Patent Publication US20200223935 (incorporated herein by reference). A nucleic acid encoding a polypeptide (e.g., a CH2 or CH3 domain) can be modified to encode 1-10 or more amino acid mutations. The encoded modifications can occur at predetermined sites, can occur randomly within selected predetermined sites, or they can occur randomly. The encoded modifications can be random, partially random, or biased with respect to the amino acid medication. The encoded modified polypeptides are then expressed using any number of systems, e.g., a display system. The display system can be, but is not limited to, a viral display system, a cell surface display system such as a yeast display system, an mRNA display system, or a polysomal display system. The modified polypeptides are then screened using known methodologies to identify transferrin receptor binders, which may be further characterized to determine binding affinity. Polypeptides identified as having affinity for TfR may be subjected to one or more additional rounds of mutation, expression, display, and selection until polypeptides have the desired affinity for TfR are identified.

In some embodiments, an engineered polypeptide comprises antibody Fc polypeptide modified to bind transferrin. An Fc polypeptide modified to bind TfR can comprise a CH2 domain modified to bind TfR or CH3 domain modified to bind TfR. The CH2 or CH3 domain modified to bind TfR can be derived from an IgG1, an IgG2, an IgG3, or an IgG4. In some embodiments, a TfR-binding region comprises a CH3 domain derived from an IgG1 modified to bind transferrin. In some embodiments, a TfR-binding region comprises a CH3 domain derived from an IgG1 modified to bind transferrin with an affinity of about 900 nM to about 10,000 nM (e.g., about 900 nM to about 2500 nM or about 900 nM to about 1300 nM).

In some embodiments, an engineered polypeptide comprises an antibody CH2 domain modified to bind TfR or an antibody CH3 domain modified to bind TfR. The CH2 or CH3 domain modified to bind TfR can be derived from an IgG1, an IgG2, an IgG3, or an IgG4. In some embodiments, a TfR-binding region comprises a CH3 domain derived from an IgG1 modified to bind transferrin. In some embodiments, a TfR-binding region comprises a CH3 domain derived from an IgG1 modified to bind transferrin with an affinity of about 900 nM to about 10,000 nM (e.g., about 900 nM to about 2500 nM or about 900 nM to about 1300 nM). CH3 domains of other immunoglobulin isotypes, e.g., IgM, IgA, IgE, IgD, etc. may be similarly modified by identifying the amino acids in those domains that correspond to the amino acid positions described herein. Modifications may also be made to corresponding domains from immunoglobulins from other species, e.g., non-human primates, monkey, mouse, rat, rabbit, dog, pig, chicken, and the like.

In some embodiments, TfR-binding region comprises a modified CH3 domain having amino acid substitutions at least six, seven, eight, nine, or ten of the positions selected from 380, 384, 386, 387, 388, 398, 390, 413, 415, 416, and 421, according to EU numbering scheme. In some embodiments, TfR-binding region comprises a modified CH3 domain having a tyrosine or phenylalanine at position 384, a threonine or asparagine at position 386, an aspartate at position 387, a tryptophan at position 388, a serine, threonine, or valine at position 389, a serine or asparagine at position 390, a threonine or serine at position 413, a serine or glutamine at position 415, a glutamate at position 416, and a phenylalanine or tyrosine at position 421. according to EU numbering scheme. In some embodiments, a TfR-binding region comprises a modified CH3 domain having a tyrosine (Y) at position 384, a threonine (T) at position 386, a glutamate (E) at position 387, a tryptophan (W) at position 388, a valine (V) at position 389, a threonine (T) at position 413, a glutamate (E) at position 415, a glutamate (E) at position 416, and a phenylalanine (F) at position 421 (according to EU numbering scheme).

In some embodiments, a TfR-binding region comprises a modified CH3 domain having any set of substitutions as listed in Table 1. In some embodiments, a TfR-binding region comprises a modified CH3 domain having any set of substitutions as listed in Table land having an amino acid sequence having at least 85% identity, at least 90% identity, or at least 95% identity to amino acids 348-454 (according to EU numbering scheme) of any of SEQ ID NOs: 8, 10, and 17-31.

TABLE 1
Amino acid substitutions in an IgG Fc domain resulting in TfR binding. Amino acid positions
are according to EU numbering scheme. Wild-type amino acids are shown for reference.

| | amino acid position | | | | | | | | | | KD for huTfR Apical |
|---|---|---|---|---|---|---|---|---|---|---|---|
| | 384 | 386 | 387 | 388 | 389 | 390 | 413 | 415 | 416 | 421 | Domain (nM) |
| wild-type | N | Q | P | E | N | N | D | S | R | N | |
| ATV35.23.3 | Y | T | E | W | V | N | T | E | E | F | 1100 |
| 35.d1.1 | Y | N | E | W | S | S | T | S | E | F | 6200 |
| 35.d1.2 | Y | T | E | W | T | S | T | S | E | F | 4300 |
| 35.d1.3 | Y | T | E | W | S | S | T | S | E | Y | 1300 |
| 35.d1.4 | F | T | E | W | S | S | S | S | E | F | 1400 |
| 35.d1.7 | Y | T | E | W | S | S | S | S | E | Y | 3000 |
| 35.d1.9 | Y | T | E | W | T | N | T | S | E | F | 8400 |
| 35.d1.10 | Y | T | E | W | V | N | T | S | E | F | 2500 |
| 35.d1.11 | Y | T | E | W | S | N | T | S | E | Y | 2500 |
| 35.d1.12 | F | T | E | W | S | N | S | S | E | F | 1600 |
| 35.d1.15 | Y | T | E | W | S | N | S | S | E | Y | 3600 |
| 35.d1.18 | F | T | E | W | S | N | S | S | E | Y | 4400 |
| 35.d1.20 | Y | T | E | W | V | N | S | S | E | F | 3600 |
| 35.d1.22 | Y | N | E | W | S | N | S | E | E | F | 5200 |
| 35.d1.23 | Y | T | E | W | T | N | S | E | E | F | 3500 |
| 35.d1.24 | Y | T | E | W | S | N | S | E | E | Y | 1100 |

In some embodiments, TfR-binding region comprises a modified CH3 domain polypeptide having at least 70% identity, at least 75% identity, at least 80% identity, at least 85% identity, at least 90% identity, or at least 95% identity to amino acids 348-454 of SEQ ID NO: 8 or 10, wherein the polypeptide has a Y at position 384, a T at position 386, an E at position 387, a W at position 388, a V at position 389, a T at position 413, an E at position 415, an E at position 416, and an F at position 421 (according to EU numbering scheme).

In some embodiments, the TfR-binding region comprises the amino acid sequence of amino acids 348-453 or 348-454 of any of SEQ ID NOs:8, 10 or 17-31. In some embodiments, the TfR-binding region comprises the amino acid sequence of amino acids 348-453 or 348-454 of SEQ ID NO:8 or 10.

In some embodiments, the TfR-binding region comprises a modified Fc polypeptide having an amino acid sequence has at least 70% identity, at least 75% identity, at least 80% identity, at least 85% identity, at least 90% identity, at least 95% identity, at least 96% identity, at least 97% identity, at least 98% identity, at least 99% identity to amino acids 228-454 of any of SEQ ID NOs: 8, 10, or 17-31, wherein the polypeptide has amino acids at positions 384, 386, 387, 388, 389, 390, 413, 415, 416, and 421 (according to EU numbering scheme) as shown in Table 1. In some embodiments, the TfR-binding region comprises a modified Fc polypeptide having an amino acid sequence has at least 70% identity, at least 75% identity, at least 80% identity, at least 85% identity, at least 90% identity, at least 95% identity, at least 96% identity, at least 97% identity, at least 98% identity, at least 99% identity to amino acids 228-454 of SEQ ID NO:8 or 10, wherein the polypeptide has a Y at position 384, a T at position 386, an E at position 387, a W at position 388, a V at position 389, a T at position 413, an E at position 415, an E at position 416, and an F at position 421 (according to EU numbering scheme).

In some embodiments, the TfR-binding region comprises a modified Fc polypeptide having an amino acid sequence has at least 70% identity, at least 75% identity, at least 80% identity, at least 85% identity, at least 90% identity, at least 95% identity, at least 96% identity, at least 97% identity, at least 98% identity, at least 99% identity to amino acids 228-454 of SEQ ID NO:8 or 10, wherein the polypeptide has an A at position 234, an A at position 235, optionally a G at position 239, a Y at position 384, a T at position 386, an E at position 387, a W at position 388, a V at position 389, a T at position 413, an E at position 415, an E at position 416, and an F at position 421 (according to EU numbering scheme).

In some embodiments, the TfR-binding region comprises the amino acid sequence of amino acids 228-453 or 228-454 of any of SEQ ID NOs: 8, 10, and 17-31. In some embodiments, the TfR-binding region comprises the amino acid sequence of amino acids 228-453 or 228-454 of SEQ ID NO:8 or 10.

### C. Fcγ Receptor (FcγR)-Binding Region

An FcγR-binding region comprises a polypeptide or a region or domain or a polypeptide or protein that specifically binds FcγR and is capable of elicit effector function activity. Effector functions include, but are not limited to, ADCP, ADCC, and CDC. Effector functions are typically mediated by binding of FcγR to the Fc domain of an immunoglobulin, such as an IgG. The described Abeta-targeting proteins can mediate at least one effector function. Effector function can aid in clearance of amyloid plaques in the brain of a subject. In some embodiments, the described Abeta-targeting proteins are configured such that binding of the Abeta-targeting protein to a TfR reduces binding of the FcγR-binding region to an FcγR. In some embodiments, the described Abeta-targeting proteins mediate effector function (bind FcγR) when bound to Abeta, but do not mediate effector function or have reduced effectiveness in mediating effector function (do not bind FcγR or have reduced binding to FcγR) when bound to TfR.

Fc receptors are proteins found on the surface of immune cells that contribute to the protective functions of the immune system. Fc-gamma receptors (FcγRs) recognize IgG-coated targets, such as opsonized antigens or immune complexes (ICs). FcγRs induce antibody-mediated cellular phagocytosis (ADCP), antibody-dependent cell-mediated cytotoxicity (ADCC), and complement-dependent cytotoxicity (CDC).

An FcγR-binding region can comprise an Fc polypeptide derived from an immunoglobulin. The Fc polypeptide can comprise all or a portion of the CH2 and CH3 domains of an immunoglobulin heavy chain. In some embodiments, the FcγR-binding region comprise an Fc polypeptide derived from an IgG1, IgG2, IgG3, or IgG4, such as a human IgG1, IgG2, IgG3, or IgG4. The Fc polypeptide can comprise all or a portion of the CH2 and CH3 domains of an immunoglobulin heavy chain. Numerous mutations (e.g., substitutions, deletions, or insertions) are known in the art for increasing or decreasing binding of an Fc polypeptide to an FcγR. In some embodiments, the FcγR-binding region comprises an Fc polypeptide that does not contain any mutations that reduce FcγR binding. The FcγR-binding region may comprise an Fc polypeptide that has one or more mutations that increase FcγR binding or increase effector function.

In some embodiments, an FcγR-binding region comprises Fc polypeptide about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a wild-type Fc polypeptide (e.g., a human IgG1, IgG2, IgG3, or IgG4 Fc polypeptide).

In some embodiments, an FcγR-binding region comprises Fc polypeptide comprising an amino acid sequence having at least 70% identity, at least 75% identity, at least 80% identity, at least 85% identity, at least 90% identity, or at least 95% identity to amino acids 228-454 of SEQ ID NO: 9, and does not contain a TfR-binding site.

In some embodiments, an FcγR-binding region comprises Fc polypeptide comprising an amino acid sequence having at least 70% identity, at least 75% identity, at least 80% identity, at least 85% identity, at least 90% identity, or at least 95% identity to amino acids 228-454 of SEQ ID NO: 9, and does not contain a TfR-binding site or any modifications that reduce FcγR binding.

In some embodiments, an FcγR-binding region comprises Fc polypeptide comprising an amino acid sequence having at least 70% identity, at least 75% identity, at least 80% identity, at least 85% identity, at least 90% identity, or at least 95% identity to amino acids 228-454 of SEQ ID NO: 9, has an S and position 366, an A and position 368 and a V at position 407, and does not contain a TfR-binding site or any modifications that reduce FcγR binding.

In some embodiments, an FcγR-binding region comprises Fc polypeptide comprising an amino acid sequence having at 100% identity to amino acids 228-453 or 228-454 of SEQ ID NO: 9.

Methods for analyzing binding affinity, binding kinetics, and cross-reactivity between FcγR-binding region and an FcγR are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet® (ForteBio, Inc., Menlo Park, CA)), and Western blot analysis. In some embodiments, ELISA is used to determine binding affinity and/or cross reactivity. Methods for performing ELISA assays are known in the art. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross reactivity.

It may be desirable to introduce modifications into an Fc polypeptide comprising an FcγR-binding region to increase effector function. One method for increasing effector function involves producing modified Fc polypeptides that are afucosylated or fucose-deficient. One approach for generating fucose-deficient Fc polypeptides is to use a fucose analog such as 2-fluorofucose (2-FF). Fucose analogs can deplete or decrease the availability of GDP-fucose, which is a substrate required by fucosyltransferases to incorporate fucose into proteins. An alternative approach for generating fucose-deficient Fc polypeptides is to employ an alpha-1,6 fucosyltransferase (FUT8) knockout cell line for expression of the Fc polypeptide. A non-limiting example of a suitable FUT8 knockout cell line is the Chinese hamster ovary (CHO) FUT8 knockout cell line available from Lonza Biologies. Furthermore, as described in Mori et al. (Biotechnol. Bioeng. (2004) 88:901-908; hereby incorporated by reference in its entirety), FUT8 small interfering RNA (siRNA) can be used to convert CHO cell lines (e.g., by constitutive expression of the FUT8 siRNA) for the production of fucose-deficient proteins.

### D. Fc Dimer

In some embodiments, the TfR-binding region and the Fcγ-binding region are provided by an Fc dimer polypeptide. TfR-binding function and FcγR-binding function can be provided on a modified Fc dimer, wherein the Fc dimer comprises a first Fc polypeptide and a second Fc polypeptide. In some embodiments, the TfR-binding region is provided on the first Fc polypeptide and the FcγR-binding region is provided on the first and second Fc polypeptides. In some embodiments, the TfR-binding region and the FcγR-binding region are provided on the first Fc polypeptide. In some embodiments, the TfR-binding region is provided on the first Fc polypeptide and the FcγR-binding region is provided on the second Fc polypeptide.

In some embodiments, the first Fc polypeptide comprises the TfR-binding region and does not contain any mutations that reduce effector function (e.g., FcγR binding, ADPC, ADCC, or CDC).

In some embodiments, the first Fc polypeptide comprises the TfR-binding region and one or more mutations that reduce effector function (e.g., FcγR binding, ADPC, ADCC, or CDC). In some embodiments, the first Fc polypeptide comprises a CH3 domain modified to bind the TfR-binding region and one or more mutations that reduce effector function.

An Fc polypeptide having “reduced binding to an FcγR” refers to a modified Fc polypeptide that contains mutations in the CH2 and/or CH3 domain which result in the Fc polypeptide having decreased affinity for an FcγR. An Fc polypeptide having reduced binding to an FcγR can have affinity for FcγR that is reduced by about 10% to about 90% compared a wild-type Fc polypeptide or an Fc polypeptide that does not contain mutations to reduce FcγR binding. In some embodiments, a mutation that reduces FcγR binding or reduces effector function, reduces FcγR binding or reduces effector function by about 15% or more, about 20% or more, about 25% or more, about 30% or more, about 35% or more, about 40% or more, about 45% or more, about 50% or more, 55% or more, about 60% or more, about 65% or more, about 70% or more, about 75% or more, about 80% or more, about 85% or more, or about 90% or more. FcγR binding may be measured using, e.g., Surface Plasmon Resonance (SPR) methods (e.g., a Biacore™ system). Alternatively, FcγR binding can be measured using a functional assay, for example, an ADCP or ADCC assay (e.g., an in vivo or in vitro assay of cell killing). The reduction of FcγR binding may be measured when the modified Fc polypeptide, Fc dimer, or Abeta-targeting protein is bound to TfR and/or Abeta. In some embodiments, an Fc polypeptide, Fc dimer or Abeta-targeting protein may have a greater reduction in binding of FcγR binding when bound to TfR than when not bound to TfR.

In some embodiments, the second Fc peptide does not contain a TfR-binding region and does not contain any modifications that reduce FcγR binding.

In some embodiments, an Fc dimer comprises a first Fc polypeptide comprising a CH3 domain modified to bind TfR and a CH2 wherein the CH2 domain does not contain any mutations that reduce effector function and a second Fc peptide comprising a CH3 domain and a CH2 domain, wherein the second Fc polypeptide does not contain a TfR-binding region.

In some embodiments, an Fc dimer comprises a first Fc polypeptide comprising a CH3 domain modified to bind TfR and a CH2 domain wherein the CH2 domain does not contain any mutations that reduce effector function and a second Fc peptide comprising a CH3 domain and a CH2 domain, wherein the second Fc polypeptide does not contain a TfR-binding region and does not contain any modifications that reduce FcγR binding.

In some embodiments, an Fc dimer comprises a first Fc polypeptide comprising a CH3 domain modified to bind TfR and a CH2 domain wherein the CH2 domain does not contain any mutations that reduce effector function and a second Fc peptide comprising a CH3 domain and a CH2 domain, wherein the second Fc polypeptide does not contain a TfR-binding region and wherein the CH2 domain contains one or more mutations that reduce effector function.

In some embodiments, the first Fc polypeptide comprises a CH3 domain modified to bind the TfR-binding region and one or more mutations that reduce effector function, and the second Fc polypeptide does not contain any modifications that reduce effector function (e.g., FcγR binding). In some embodiments, an Fc dimer comprises a first Fc polypeptide comprising a CH3 domain modified to bind TfR and a CH2 domain modified to reduce effector function (e.g., FcγR binding, ADPC, ADCC, or CDC) and a second Fc peptide comprising a CH3 domain and a CH2 domain, wherein the second Fc polypeptide does not contain a TfR-binding region and does not contain any modifications that reduce FcγR binding. Antibody constructs having this Fc dimer configuration are described in WO2019140050. An Abeta-targeting protein having an Fc dimer in this configuration does not bind FcγR or mediate effector function or has reduced FcγR binding or reduced effector function when bound to TR, but is able to bind FcγR and mediate effector function when not bound to TfR. Decreased binding to FcγR and decreased effector function when bound to TfR leads to decreased depletion of reticulocytes. Such dimers also provide for effector function when the Abeta-targeting protein is bound to Abeta. Effector function when bound to Abeta (e.g., amyloid plaques or fibrils) can facilitate reduction in amyloid plaques or fibrils in a subject.

In some embodiments, the described Abeta-targeting proteins do not cause a substantial reduction in reticulocytes (e.g., a reduction in bone marrow reticulocytes or circulating reticulocytes). In some embodiments, administration of a described Abeta-targeting protein to a subject causes a less that than 80%, 75%, 70%, 65%, 60%, 55%, 50%, 45%, 40%, 35%, 30%, 25%, 20%, 15%, 10%, 8%, 5%, 3%, 2%, or 1% the reduction in bone marrow or circulating reticulocytes compared to the number of bone marrow or circulating reticulocytes in the subject prior to administration of the Abeta-targeting protein, compared to a control subject that received a control protein, or compared to a predetermined control.

Fc polypeptide mutations that modulate effector function include, but are not limited to, substitutions at positions: 234 (e.g., L234A), 235 (e.g., L235A), and 329 (e.g., P329G) (IgG1 Fc); 228 (e.g., S228P) and 235 (e.g., L235E) (IgG4 Fc); 234 (e.g., L234A) and 237 (e.g., G237A) (IgG1 Fc); 234 (e.g., L234A), 235 (e.g., L235A), and 237 (e.g., G237A) (IgG1 Fc); 234 (e.g., V234A) and 237 (e.g., G237A) (IgG2 Fc); 235 (e.g., L235A), 237 (e.g., G237A), and 318 (e.g., E318A) (IgG4 Fc); and 228 (e.g., S228P) and 236 (e.g., L236E) (IgG4 Fc) (according to EU numbering scheme).

In some embodiments, the first Fc polypeptide comprises one or more of L234A, L235A, or P329G substitutions. In some embodiments, the first Fc polypeptide comprises L234A and L235A substitutions. In some embodiments, the first Fc polypeptide comprises a P329G substitution. In some embodiments, the first Fc polypeptide comprises L234A, L235A, and P329G substitutions.

In some embodiments, an Fc dimer comprises: (a) a first Fc polypeptide comprising a TfR-binding region, and amino acid modifications L234A and L235A, according to EU numbering scheme, and (b) a second Fc polypeptide that does not contain a TfR-binding site or any modifications that reduce FcγR binding.

In some embodiments, an Fc dimer comprises: (a) a first Fc polypeptide comprising a TfR-binding region, and amino acid modifications L234A, L235A and P329G, according to EU numbering scheme, and (b) a second Fc polypeptide that does not contain a TfR-binding site or any modifications that reduce FcγR binding.

Fc dimer can be provided as a single chain or as two polypeptides. The first Fc polypeptide or the second Fc polypeptide in an Fc dimer may have one or more mutations that facilitate heterodimerization of two Fc polypeptides. Any Fc mutations known in the art that facilitate, promote, or enhance heterodimerization can be used with the described Fc polypeptides. Such mutations include, but are not limited to, knob and hole mutations. In some embodiments, the first Fc polypeptide comprises a knob mutation and the second Fc polypeptide comprises hole mutations. In some embodiments, the first Fc polypeptide comprises hole mutations and the second Fc polypeptide comprises a knob mutation. The knob mutation can comprise a T366W substitution (according to EU numbering scheme). The hole mutation can comprise T366S, T368A, and Y407V substitutions (according to EU numbering scheme). In some embodiments, the first Fc polypeptide comprises a T366W substitution, and the second Fc polypeptide comprises T366S, T368A, and Y407V substitutions. In some embodiments, the first Fc polypeptide comprises T366S, T368A, and Y407V substitutions, and the second Fc polypeptide comprises a T366W substitution.

In some embodiments, an Fc dimer comprises: (a) a first Fc polypeptide comprising a TfR-binding site that specifically binds TfR, amino acid modifications L234A and L235A, and hole mutations T366S, L368A, and Y407V (according to EU numbering scheme), and (b) a second Fc polypeptide that comprises a knob mutation T366W (according to EU numbering scheme), and does not contain a TfR-binding site or any modifications that reduce FcγR binding.

In some embodiments, an Fc dimer comprises: (a) a first Fc polypeptide comprising a TfR-binding site that specifically binds TfR, amino acid modifications L234A, L235A, and P329G, and hole mutations T366S, L368A, and Y407V (according to EU numbering scheme), and (b) a second Fc polypeptide that comprises a knob mutation T366W (according to EU numbering scheme), and does not contain a TfR-binding site or any modifications that reduce FcγR binding.

In some embodiments, an Fc dimer comprises: (a) a first Fc polypeptide comprising a TfR-binding site that specifically binds TfR, amino acid modifications L234A and L235A, and knob mutations T366W (according to EU numbering scheme), and (b) a second Fc polypeptide that comprises a hole mutations T366S, L368A, and Y407V (according to EU numbering scheme), and does not contain a TfR-binding site or any modifications that reduce FcγR binding.

In some embodiments, an Fc dimer comprises: (a) a first Fc polypeptide comprising a TfR-binding site that specifically binds TfR, amino acid modifications L234A, L235A, and P329G, and knob mutations T366W (according to EU numbering scheme), and (b) a second Fc polypeptide that comprises a hole mutations T366S, L368A, and Y407V (according to EU numbering scheme), and does not contain a TAR-binding site or any modifications that reduce FcγR binding.

In some embodiments, an Fc dimer comprises (a) a first Fc polypeptide comprising an amino acid sequence having at least 70% identity, at least 75% identity, at least 80% identity, at least 85% identity, at least 90% identity, or at least 95% identity to amino acids 228-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1; and (b) a second Fc polypeptide comprising an amino acid sequence having at least 70% identity, at least 75% identity, at least 80% identity, at least 85% identity, at least 90% identity, or at least 95% identity to amino acids 228-454 of SEQ ID NO: 9, wherein the second Fc polypeptide has an S and position 366, an A and position 368 and a V at position 407, and does not contain a TfR-binding site or any modifications that reduce FcγR binding.

In some embodiments, an Fc dimer comprises a first Fc polypeptide comprising an amino acid sequence having 100% identity to amino acids 225-453 or 228-454 of any of SEQ ID NO:8, 10, and 17-31, and a second Fc polypeptide comprising an amino acid sequence having at 100% identity to amino acids 228-453 or 228-454 of SEQ ID NO: 9. In some embodiments, an Fc dimer comprises a first Fc polypeptide comprising an amino acid sequence having 100% identity to amino acids 228-453 of SEQ ID NOs: 8, 10, and 17-31, and a second Fc polypeptide comprising an amino acid sequence having at 100% identity to amino, acids 228-453 of SEQ ID NO: 9. In some embodiments, the first Fc polypeptide comprises: (a) an A at position 234, and A at position 235, (b) a G at position 329, (c) an A at position 234, and A at position 235, and a G at position 329, (d) a W at position 366, (e) an A at position 234, and A at position 235, and a W at position 366, (f)) a G at position 329 and a W at position 366, or (g) an A at position 234, and A at position 235, a G at position 329, and a W at position 366.

In some embodiments, an Fc dimer comprises (a) a first Fc polypeptide comprising an amino acid sequence having at least 70% identity, at least 75% identity, at least 80% identity, at least 85% identity, at least 90% identity, or at least 95% identity to amino acids 228-454 of SEQ ID NO:8, wherein the first Fc polypeptide has an A at position 234, and A at position 235, a G at position 329, a W at position 366, a Y at position 384, a T at position 386, an E at position 387, a W at position 388, a V at position 389, a T at position 413, an E at position 415, an E at position 416, and an F at position 421 (according to EU numbering scheme); and (b) a second Fc polypeptide comprising an amino acid sequence having at least 70% identity, at least 75% identity, at least 80% identity, at least 85% identity, at least 90% identity, or at least 95% identity to amino acids 228-454 of SEQ ID NO: 9, wherein the second Fc polypeptide has an S and position 366, an A and position 368 and a V at position 407, and does not contain a TfR-binding site or any modifications that reduce FcγR binding.

In some embodiments, an Fc dimer comprises a first Fc polypeptide comprising an amino acid sequence having 100% identity to amino acids 228-454 of SEQ ID NO:8, and a second Fc polypeptide comprising an amino acid sequence having at 100% identity to amino acids 228-454 of SEQ ID NO: 9. In some embodiments, an Fc dimer comprises a first Fc polypeptide comprising an amino acid sequence having 100% identity to amino acids 228-453 of SEQ ID NO:8, and a second Fc polypeptide comprising an amino acid sequence having at 100% identity to amino acids 228-453 of SEQ ID NO: 9.

In some embodiments, an Fc dimer comprises (a) a first Fc polypeptide comprising an amino acid sequence having at least 70% identity, at least 75% identity, at least 80% identity, at least 85% identity, at least 90% identity, or at least 95% identity to amino acids 228-454 of SEQ ID NO: 10, wherein the first Fc polypeptide has an A at position 234, and A at position 235, a W at position 366, a Y at position 384, a T at position 386, an E at position 387, a W at position 388, a V at position 389, a T at position 413, an E at position 415, an E at position 416, and an F at position 421 (according to EU numbering scheme); and (b) a second Fc polypeptide comprising an amino acid sequence having at least 70% identity, at least 75% identity, at least 80% identity, at least 85% identity, at least 90% identity, or at least 95% identity to amino acids 228-454 to SEQ ID NO: 9, wherein the second Fc polypeptide has an S and position 366, an A and position 368 and a V at position 407, and does not contain a TfR-binding site or any modifications that reduce FcγR binding.

In some embodiments, an Fc dimer comprises a first Fc polypeptide comprising an amino acid sequence having 100% identity to amino acids 228-454 of SEQ ID NO:10, and a second Fc polypeptide comprising an amino acid sequence having at 100% identity to amino acids 228-454 of SEQ ID NO: 9. In some embodiments, an Fc dimer comprises a first Fc polypeptide comprising an amino acid sequence having 100% identity to amino acids 228-453 of SEQ ID NO:10, and a second Fc polypeptide comprising an amino acid sequence having at 100% identity to amino acids 228-453 of SEQ ID NO: 9.

Any of the described Fc dimers can have an FcRn binding site or one or more mutations that increase or decrease FcRn binding. The term “FcRn” refers to the neonatal Fc receptor. Binding of Fc polypeptides to FcRn reduces clearance and increases serum half-life of the Fc polypeptide. Any of the described Fc dimers may have one or more mutations that increase the half-life or serum half-life (e.g., stability) of the Abeta-targeting protein. Such mutations can be any of the mutations known in the art to increase the half-life or serum half-life of an IgG. Mutations known to increase half-life of IgG include, but are not limited to, mutations at one or more of positions T250, M252, S254, T256, T307, E380, M428, and N434 (according to EU numbering scheme). In some embodiments, the first Fc polypeptide and/or the second polypeptide in an Fc dimer may comprise: (a) one or more of M252Y, S254T, and T256E substitutions; (b) M428L and/or N434S substitutions; (c) one or more of T307A, E380A, and N434A substitutions; (d) T250Q and/or M428L substitutions; (e) M428L and/or N434S substitutions; and/or (f) N434S and/or N434A substitutions. Mutations to modulate FcRn binding may be present in the first PC polypeptide of an Fc dimer, in the second Fc polypeptide of an Fc dimer, or in both polypeptides of an Fc dimer. Mutations to increase half-life or serum half-life may be present in the first PC polypeptide of an Fc dimer, in the second Fc polypeptide of an Fc dimer, or in both polypeptides of an Fc dimer.

In some embodiments, an Fc dimer comprises: (a) a first Fc polypeptide comprising a TfR-binding region, amino acid modifications L234A and L235A and optionally P329G, and amino acid modification N434S with or without M428L (according to EU numbering scheme), and (b) a second Fc polypeptide that does not contain a TfR-binding site or any modifications that reduce FcγR binding.

In some embodiments, an Fc dimer comprises: (a) a first Fc polypeptide comprising a TfR-binding region, amino acid modifications L234A and L235A and optionally P329G, and amino acid modification N434S with or without M428L (according to EU numbering scheme), and (b) a second Fc polypeptide that comprises amino acid modification N434S with or without M428L and does not contain a TfR-binding site or any modifications that reduce FcγR binding.

In some embodiments, an Fc dimer or an Fc polypeptide as described herein further comprises a partial or full hinge region. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:16). In further embodiments, the Fc polypeptide, which may comprise a hinge or partial hinge region, is further fused to the polypeptide comprising an Abeta-binding region.

In some embodiments, the Fc dimer or an Fc polypeptide as described herein is fused to a polypeptide comprising an Abeta-binding region via a linker. The linker can be, but is not limited to, a peptide linker (e.g., a hinge region). The peptide linker may be configured such that it allows for the rotation of the variable region and the Fc polypeptide or Fc dime to each other; and/or is resistant to digestion by proteases. In some embodiments, the linker may be a flexible linker, e.g., containing amino acids such as Gly, Asn, Ser, Thr, Ala, and the like. Such linkers are designed using known parameters. For example, the linker may have repeats, such as Gly-Ser or (Gly)ₘ(Ser)ₙ repeats.

In some embodiments, an Abeta-targeting protein comprises an Abeta-binding region comprising heavy chain Fab region and a light chain Fab region, a TfR-binding region comprising a first Fc polypeptide, and an FcγR-binding region comprising a second Fc polypeptide, wherein the first and second Fc polypeptides dimerize. The first or the second Fc polypeptide may be linked to the heavy chain Fab region.

In some embodiments, an Abeta-targeting protein comprises two Abeta-binding regions wherein each Abeta-binding region comprises a heavy chain Fab region and a light chain Fab region, a TfR-binding region comprising a first Fc polypeptide, and an FcγR-binding region comprising a second Fc polypeptide, wherein the first and second Fc polypeptides dimerize, and where the first Fc polypeptide is linked to one heavy chain Fab region and the second Fc polypeptide is linked to the other heavy chain Fab region. A person skilled in the art will recognize that this configuration resembles an immunoglobulin.

In some embodiments, an Abeta-targeting protein comprises an Abeta-binding region linked to an Fc dimer, wherein the Fc dimer comprises:
(a) a first Fc polypeptide comprising a TfR-binding region that specifically binds a TfR with an affinity of about 900 nM to about 10,000 nM (e.g., about 900 nM to about 2500 nM or about 900 nM to about 1300 nM); and
(b) a second Fc polypeptide comprising an FcγR-binding region.

In some embodiments, an Abeta-targeting protein comprises an Abeta-binding region linked to an Fc dimer, wherein the Fc dimer comprises:
(a) a first Fc polypeptide comprising a TfR-binding region that specifically binds a TfR with an affinity of about 900 nM to about 10,000 nM (e.g., about 900 nM to about 2500 nM or about 900 nM to about 1300 nM); and
(b) a second Fc polypeptide comprising an FcγR-binding region wherein the second Fc polypeptide does not contain a TfR-binding region.

In some embodiments, an Abeta-targeting protein comprises an Abeta-binding region linked to an Fc dimer, wherein the Fc dimer comprises:
(a) a first Fc polypeptide comprising a TfR-binding region that specifically binds a TfR with an affinity of about 900 nM to about 10,000 nM (e.g., about 900 nM to about 2500 nM or about 900 nM to about 1300 nM) and; and
(b) a second Fc polypeptide comprising an FcγR-binding region wherein the second Fc polypeptide does not contain a TfR-binding region;
wherein the first Fc polypeptide comprises one or more amino acid substitutions that reduce binding to an FcγR; and
wherein the second Fc polypeptide does not contain any modifications that reduce FcγR binding.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1; and
(b) a second Fc polypeptide comprising an FcγR-binding region.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, or at least 95% identity to amino acids 348-454 of SEQ ID NO:8 or 10, wherein the first Fc polypeptide has a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an FcγR-binding region.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1; and
(b) a second Fc polypeptide comprising an FcγR-binding region wherein the second Fc polypeptide does not contain a TfR-binding region.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, or at least 95% identity to amino acids 348-454 of SEQ ID NO:8 or 10, wherein the first Fc polypeptide has a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an FcγR-binding region wherein the second Fc polypeptide does not contain a TfR-binding region.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1; and
(b) a second Fc polypeptide comprising an FcγR-binding region wherein the second Fc polypeptide does not contain a TfR-binding region and does not contain any modifications that reduce FcγR binding.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, or at least 95% identity to amino acids 348-454 of SEQ ID NO:8 or 10, wherein the first Fc polypeptide has a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an FcγR-binding region wherein the second Fc polypeptide does not contain a TfR-binding region and does not contain any modifications that reduce FcγR binding.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and one or more amino acid substitutions that reduce binding to an FcγR; and
(b) a second Fc polypeptide comprising an FcγR-binding region.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, or at least 95% identity to amino acids 348-454 of SEQ ID NO:8 or 10, wherein the first Fc polypeptide has a Y at position 384, a T at position 386, an E at position 387, a W at position 388, a V at position 389, a T at position 413, an E at position 415, an E at position 416, and an F at position 421 (according to EU numbering scheme) and one or more amino acid substitutions that reduce binding to an FcγR; and
(b) a second Fc polypeptide comprising an FcγR-binding region.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and one or more amino acid substitutions that reduce binding to an FcγR; and
(b) a second Fc polypeptide comprising an FcγR-binding region, wherein the second Fc polypeptide does not contain a TfR-binding region.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, or at least 95% identity to amino acids 348-454 of SEQ ID NO:8 or 10, wherein the first Fc polypeptide has a Y at position 384, a T at position 386, an E at position 387, a W at position 388, a V at position 389, a T at position 413, an E at position 415, an E at position 416, and an F at position 421 (according to EU numbering scheme) and one or more amino acid substitutions that reduce binding to an FcγR; and
(b) a second Fc polypeptide comprising an FcγR-binding region, wherein the second Fc polypeptide does not contain a TfR-binding region.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and one or more amino acid substitutions that reduce binding to an FcγR; and
(b) a second Fc polypeptide comprising an FcγR-binding region, wherein the second Fc polypeptide does not contain a TfR-binding region and does not contain any modifications that reduce FcγR binding.

In some embodiments, an Abeta-targeting protein comprises an Abeta-binding region linked to an Fc dimer, wherein the Fc dimer comprises:
(a) a first Fc polypeptide comprising a modified CH3 domain polypeptide having at least 70% identity, at least 75% identity, at least 80% identity, at least 85% identity, at least 90% identity, or at least 95% identity to amino acids 348-454 of SEQ ID NO:8 or 10, wherein the first Fc polypeptide has a Y at position 384, a T at position 386, an E at position 387, a W at position 388, a V at position 389, a T at position 413, an E at position 415, an E at position 416, and an F at position 421 (according to EU numbering scheme) and one or more amino acid substitutions that reduce binding to an FcγR; and
(b) a second Fc polypeptide comprising an FcγR-binding region, wherein the second Fc polypeptide does not contain a TfR-binding region and does not contain any modifications that reduce FcγR binding.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and an A at position 234, and an A at position 235; and
(b) a second Fc polypeptide comprising an FcγR-binding region.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:8 or 10, wherein the first Fc polypeptide has an A at position 234, an A at position 235, a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an FcγR-binding region.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and an A at position 234, an A at position 235, and a G at position 329; and
(b) a second Fc polypeptide comprising an FcγR-binding region.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:8 or 10, wherein the first Fc polypeptide has an A at position 234, an A at position 235, a G at position 329, a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an FcγR-binding region.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and an A at position 234, and an A at position 235; and
(b) a second Fc polypeptide comprising an FcγR-binding region, wherein the second Fc polypeptide does not contain a TfR-binding region.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:8 or 10, wherein the first Fc polypeptide has an A at position 234, an A at position 235, a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an FcγR-binding region, wherein the second Fc polypeptide does not contain a TfR-binding region.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and an A at position 234, an A at position 235, and a G at position 329; and
(b) a second Fc polypeptide comprising an FcγR-binding region, wherein the second Fc polypeptide does not contain a TfR-binding region.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:8 or 10, wherein the first Fc polypeptide has an A at position 234, an A at position 235, a G at position 329, a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an FcγR-binding region, wherein the second Fc polypeptide does not contain a TfR-binding region.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and an A at position 234, and an A at position 235; and
(b) a second Fc polypeptide comprising an FcγR-binding region, wherein the second Fc polypeptide does not contain a TfR-binding region and does not contain any modifications that reduce FcγR binding.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:8 or 10, wherein the first Fc polypeptide has an A at position 234, an A at position 235, a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an FcγR-binding region, wherein the second Fc polypeptide does not contain a TfR-binding region and does not contain any modifications that reduce FcγR binding.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and an A at position 234, an A at position 235, and a G at position 329; and
(b) a second Fc polypeptide comprising an FcγR-binding region, wherein the second Fc polypeptide does not contain a TfR-binding region and does not contain any modifications that reduce FcγR binding.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:8 or 10, wherein the first Fc polypeptide has an A at position 234, an A at position 235, a G at position 329, a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an FcγR-binding region, wherein the second Fc polypeptide does not contain a TfR-binding region and does not contain any modifications that reduce FcγR binding.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, at least 95%, or 100% identity to amino acids 348-454 of any of SEQ ID NOs: 8, 10, and 17-31, wherein the polypeptide has the amino acids at positions 384, 386, 387, 388, 389, 390, 413, 415, 416, and 421 (according to EU numbering scheme) as shown in Table 1 and an A at position 234, an A at position 235, optionally a G at position 329, and W at position 366; and
(b) a second Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:9, wherein the second Fc polypeptide has an S at position 366, an A at position 368, and a V at position 407 (according to EU numbering scheme), comprises an FcγR-binding region, and does not contain a TfR-binding region.

In some embodiments, an Abeta-targeting protein comprises an Abeta-binding region linked to an Fc dimer, wherein the Fc dimer comprises:
(a) a first Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:8 or 10, wherein the first Fc polypeptide has an A at position 234, an A at position 235, optionally a G at position 329, a W at position 366, a Y at position 384, a T at position 386, an E at position 387, a W at position 388, a V at position 389, a T at position 413, an E at position 415, an E at position 416, and an F at position 421 (according to EU numbering scheme); and
(b) a second Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:9, wherein the second Fc polypeptide has an S at position 366, an A at position 368, and a V at position 407 (according to EU numbering scheme), comprises an FcγR-binding region, and does not contain a TfR-binding region.

In some embodiments, an Abeta-targeting protein comprises an Abeta-binding region linked to an Fc dimer, wherein the Fc dimer comprises:
(a) a first Fc polypeptide comprising the amino acid sequence of amino acids 348-453 or 348-454 of any of SEQ ID NOs: 8, 10, and 17-31; and
(b) a second Fc polypeptide comprising the amino acid sequence of amino acids 348-453 or 348-454 of SEQ ID NO:9.

In some embodiments, an Abeta-targeting protein comprises an Abeta-binding region linked to an Fc dimer, wherein the Fc dimer comprises:
(a) a first Fc polypeptide comprising the amino acid sequence of amino acids 348-453 or 348-454 of SEQ ID NO:8; and
(b) a second Fc polypeptide comprising the amino acid sequence of amino acids 348-453 or 348-454 of SEQ ID NO:9.

In some embodiments, an Abeta-targeting protein comprises an Abeta-binding region linked to an Fc dimer, wherein the Fc dimer comprises:
(a) a first Fc polypeptide comprising the amino acid sequence of amino acids 348-453 or 348-454 of SEQ ID NO:10; and
(b) a second Fc polypeptide comprising the amino acid sequence of amino acids 348-453 or 348-454 of SEQ ID NO:9.

In some embodiments, an Abeta-targeting protein comprises an Abeta-binding region linked to an Fc dimer, wherein the Fc dimer comprises:
(a) a first Fc polypeptide comprising the amino acid sequence of amino acids 228-453 or 228-454 of any of SEQ ID NOs: 8, 10, and 1-31; and
(b) a second Fc polypeptide comprising the amino acid sequence of amino acids 228-453 or 228-454 of SEQ ID NO:9.

In some embodiments, an Abeta-targeting protein comprises an Abeta-binding region linked to an Fc dimer, wherein the Fc dimer comprises:
(a) a first Fc polypeptide comprising the amino acid sequence of amino acids 228-453 or 228-454 of SEQ ID NO:8; and
(b) a second Fc polypeptide comprising the amino acid sequence of amino acids 228-453 or 228-454 of SEQ ID NO:9.

In some embodiments, an Abeta-targeting protein comprises an Abeta-binding region linked to an Fc dimer, wherein the Fc dimer comprises:
(a) a first Fc polypeptide comprising the amino acid sequence of amino acids 228-453 or 228-454 of SEQ ID NO:10; and
(b) a second Fc polypeptide comprising the amino acid sequence of amino acids 228-453 or 228-454 of SEQ ID NO:9.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each comprise a VL comprising a VLCDR1 having the amino acid sequence of SEQ ID NO:4, a VLCDR2 having the amino acid sequence of SEQ ID NO:5, and a VLCDR3 having the amino acid sequence of SEQ ID NO:6;
(b) the first heavy chain polypeptide comprises
   (i) a VH comprising a VHCDR1 having the amino acid sequence of SEQ ID NO: 1, a VHCDR2 having the amino acid sequence of SEQ ID NO:2, and a VHCDR3 having the amino acid sequence of SEQ ID NO:3, and
   (ii) a first Fc polypeptide comprising a TfR-binding region that specifically binds a TfR with an affinity of about 900 nM to about 10,000 nM (e.g., about 900 nM to about 2500 nM or about 900 nM to about 1300 nM); and
(c) the second heavy chain polypeptide comprises
   (i) a VH comprising a VHCDR1 having the amino acid sequence of SEQ ID NO: 1, a VHCDR2 having the amino acid sequence of SEQ ID NO:2, and a VHCDR3 having the amino acid sequence of SEQ ID NO:3, and
   (ii) a second Fc polypeptide comprising an FcγR-binding region,
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each comprising a VL comprising an amino acid sequence having at least 85% identity, at least 90% identity, or at least 95% identity to amino acids 1-109 of SEQ ID NO:7, a VLCDR1 having the amino acid sequence of SEQ ID NO:4, a VLCDR2 having the amino acid sequence of SEQ ID NO:5, and a VLCDR3 having the amino acid sequence of SEQ ID NO:6;
(b) the first heavy chain polypeptide comprises
   (i) a VH comprising an amino acid sequence having at least 85% identity, at least 90% identity, at least 95%, or 100% identity to amino acids 1-120 of any of SEQ ID NOs: 8, 10, and 17-31, a VHCDR1 having the amino acid sequence of SEQ ID NO:1, a VHCDR2 having the amino acid sequence of SEQ ID NO:2, and a VHCDR3 having the amino acid sequence of SEQ ID NO:3 and comprising, and
   (ii) a first Fc polypeptide comprising a modified CH3 domain polypeptide having at least 70% identity, at least 75% identity, at least 80% identity, at least 85% identity, at least 90% identity, at least 95% or 100% identity to amino acids 348-454 of any of SEQ ID NOs: 8, 10, and 17-31 wherein the polypeptide has the amino acids at positions 384, 386, 387, 388, 389, 390, 413, 415, 416, and 421 (according to EU numbering scheme) as shown in Table 1;
   (iii) optionally wherein the CH3 domain polypeptide comprises one or more of: an A at position 234, an A at position 235, a G at position 329, and W at position 366
(c) the second heavy chain polypeptide comprises
   (i) a VH comprising an amino acid sequence having at least 85% identity, at least 90% identity, or at least 95% identity to amino acids 1-120 of SEQ ID NO:8 or 10, a VHCDR1 having the amino acid sequence of SEQ ID NO:1, a VHCDR2 having the amino acid sequence of SEQ ID NO:2, and a VHCDR3 having the amino acid sequence of SEQ ID NO: 3 and comprising, and
   (ii) a second Fc polypeptide comprising an FcγR-binding region, optionally wherein the second Fc polypeptide does not contain a TfR-binding region, optionally wherein the second Fc polypeptide comprises an amino acid sequence having at least 70% identity, at least 75% identity, at least 80% identity, at least 85% identity, at least 90% identity, or at least 95% identity to amino acids 228-453 or 228-454 of SEQ ID NO:9, optionally wherein the second Fc polypeptide has an S at position 366, an A at position 368, and a V at position 407 (according to EU numbering scheme);
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each comprising a VL comprising an amino acid sequence having at least 85% identity, at least 90% identity, or at least 95% identity to amino acids 1-109 of SEQ ID NO:7, a VLCDR1 having the amino acid sequence of SEQ ID NO:4, a VLCDR2 having the amino acid sequence of SEQ ID NO:5, and a VLCDR3 having the amino acid sequence of SEQ ID NO:6;
(b) the first heavy chain polypeptide comprises
   (i) a VH comprising an amino acid sequence having at least 85% identity, at least 90% identity, or at least 95% identity to amino acids 1-120 of SEQ ID NO:8 or 10, a VHCDR1 having the amino acid sequence of SEQ ID NO:1, a VHCDR2 having the amino acid sequence of SEQ ID NO:2, and a VHCDR3 having the amino acid sequence of SEQ ID NO: 3 and comprising, and
   (ii) a first Fc polypeptide comprising a modified CH3 domain polypeptide having at least 70% identity, at least 75% identity, at least 80% identity, at least 85% identity, at least 90% identity, or at least 95% identity to amino acids 348-454 of SEQ ID NO:8 or 10, wherein the first Fc polypeptide has a Y at position 384, a T at position 386, an E at position 387, a W at position 388, a V at position 389, a T at position 413, an E at position 415, an E at position 416, and an F at position 421 (according to EU numbering scheme); and
(c) the second heavy chain polypeptide comprises
   (i) a VH comprising an amino acid sequence having at least 85% identity, at least 90% identity, or at least 95% identity to amino acids 1-120 of SEQ ID NO:8 or 10, a VHCDR1 having the amino acid sequence of SEQ ID NO:1, a VHCDR2 having the amino acid sequence of SEQ ID NO:2, and a VHCDR3 having the amino acid sequence of SEQ ID NO: 3 and comprising, and
   (ii) a second Fc polypeptide comprising an FcγR-binding region,
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each comprising a VL comprising an amino acid sequence having at least 85% identity, at least 90% identity, or at least 95% identity to amino acids 1-109 of SEQ ID NO:7, a VLCDR1 having the amino acid sequence of SEQ ID NO:4, a VLCDR2 having the amino acid sequence of SEQ ID NO:5, and a VLCDR3 having the amino acid sequence of SEQ ID NO:6;
(b) the first heavy chain polypeptide comprises
   (i) a VH comprising an amino acid sequence having at least 85% identity, at least 90% identity, or at least 95% identity to amino acids 1-120 of SEQ ID NO:8 or 10, a VHCDR1 having the amino acid sequence of SEQ ID NO:1, a VHCDR2 having the amino acid sequence of SEQ ID NO:2, and a VHCDR3 having the amino acid sequence of SEQ ID NO: 3 and comprising, and
   (ii) a first Fc polypeptide comprising a modified CH3 domain polypeptide having at least 70% identity, at least 75% identity, at least 80% identity, at least 85% identity, at least 90% identity, or at least 95% identity to amino acids 348-454 of SEQ ID NO:8 or 10, wherein the first Fc polypeptide has a Y at position 384, a T at position 386, an E at position 387, a W at position 388, a V at position 389, a T at position 413, an E at position 415, an E at position 416, and an F at position 421 (according to EU numbering scheme) and one or more amino acid substitutions that reduce binding to an FcγR; and
(c) the second heavy chain polypeptide comprises
   (i) a VH comprising an amino acid sequence having at least 85% identity, at least 90% identity, or at least 95% identity to amino acids 1-120 of SEQ ID NO:8 or 10, a VHCDR1 having the amino acid sequence of SEQ ID NO:1, a VHCDR2 having the amino acid sequence of SEQ ID NO:2, and a VHCDR3 having the amino acid sequence of SEQ ID NO: 3 and comprising, and
   (ii) a second Fc polypeptide comprising an FcγR-binding region, wherein the second Fc polypeptide does not contain a TfR-binding region,
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each comprising a VL comprising an amino acid sequence having at least 85% identity, at least 90% identity, or at least 95% identity to amino acids 1-109 of SEQ ID NO:7, a VLCDR1 having the amino acid sequence of SEQ ID NO:4, a VLCDR2 having the amino acid sequence of SEQ ID NO:5, and a VLCDR3 having the amino acid sequence of SEQ ID NO:6;
(b) the first heavy chain polypeptide comprises
   (i) a VH comprising an amino acid sequence having at least 85% identity, at least 90% identity, or at least 95% identity to amino acids 1-120 of SEQ ID NO:8 or 10, a VHCDR1 having the amino acid sequence of SEQ ID NO:1, a VHCDR2 having the amino acid sequence of SEQ ID NO:2, and a VHCDR3 having the amino acid sequence of SEQ ID NO: 3 and comprising, and
   (ii) a first Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:8 or 10, wherein the first Fc polypeptide has an A at position 234, an A at position 235, optionally a G at position 329, a Y at position 384, a T at position 386, an E at position 387, a W at position 388, a V at position 389, a T at position 413, an E at position 415, an E at position 416, and an F at position 421 (according to EU numbering scheme); and
(c) the second heavy chain polypeptide comprises
   (i) a VH comprising an amino acid sequence having at least 85% identity, at least 90% identity, or at least 95% identity to amino acids 1-120 of SEQ ID NO:8 or 10, a VHCDR1 having the amino acid sequence of SEQ ID NO:1, a VHCDR2 having the amino acid sequence of SEQ ID NO:2, and a VHCDR3 having the amino acid sequence of SEQ ID NO: 3 and comprising, and
   (ii) a second Fc polypeptide comprising an FcγR-binding region, wherein the second Fc polypeptide does not contain a TfR-binding region,
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each comprising a VL comprising an amino acid sequence having at least 85% identity, at least 90% identity, or at least 95% identity to amino acids 1-109 of SEQ ID NO:7, a VLCDR1 having the amino acid sequence of SEQ ID NO:4, a VLCDR2 having the amino acid sequence of SEQ ID NO:5, and a VLCDR3 having the amino acid sequence of SEQ ID NO:6;
(b) the first heavy chain polypeptide comprises
   (i) a VH comprising an amino acid sequence having at least 85% identity, at least 90% identity, or at least 95% identity to amino acids 1-120 of SEQ ID NO:8 or 10, a VHCDR1 having the amino acid sequence of SEQ ID NO:1, a VHCDR2 having the amino acid sequence of SEQ ID NO:2, and a VHCDR3 having the amino acid sequence of SEQ ID NO: 3 and comprising, and
   (ii) a first Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:8 or 10, wherein the first Fc polypeptide has an A at position 234, an A at position 235, optionally a G at position 329, a W at position 366, a Y at position 384, a T at position 386, an E at position 387, a W at position 388, a V at position 389, a T at position 413, an E at position 415, an E at position 416, and an F at position 421 (according to EU numbering scheme); and
(c) the second heavy chain polypeptide comprises
   (i) a VH comprising an amino acid sequence having at least 85% identity, at least 90% identity, or at least 95% identity to amino acids 1-120 of SEQ ID NO:8 or 10, a VHCDR1 having the amino acid sequence of SEQ ID NO:1, a VHCDR2 having the amino acid sequence of SEQ ID NO:2, and a VHCDR3 having the amino acid sequence of SEQ ID NO: 3 and comprising, and
   (ii) a second Fc polypeptide comprising an amino acid sequence having at least 70% identity, at least 75% identity, at least 80% identity, at least 85% identity, at least 90% identity, or at least 95% identity to amino acids 228-453 or 228-454 of SEQ ID NO:9, wherein the second Fc polypeptide has an S at position 366, an A at position 368, and a V at position 407 (according to EU numbering scheme), comprises an FcγR-binding region, and does not contain a TfR-binding region,
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each comprise the amino acid sequence of SEQ ID NO:7;
(b) the first heavy chain polypeptide comprises the amino acid sequence of amino acids 1-453 or 1-454 of any of SEQ ID NOs: 8, 10, and 17-31; and
(c) the second heavy chain polypeptide comprises the amino acid sequence of amino acids 1-453 or 1-454 of SEQ ID NO:9,
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each comprise the amino acid sequence of SEQ ID NO:7;
(b) the first heavy chain polypeptide comprises the amino acid sequence of amino acids 1-453 or 1-454 of SEQ ID NO:8; and
(c) the second heavy chain polypeptide comprises the amino acid sequence of amino acids 1-453 or 1-454 of SEQ ID NO:9,
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each comprise the amino acid sequence of SEQ ID NO:7;
(b) the first heavy chain polypeptide comprises the amino acid sequence of amino acids 1-453 or 1-454 of SEQ ID NO:10; and
(c) the second heavy chain polypeptide comprises the amino acid sequence of amino acids 1-453 or 1-454 of SEQ ID NO:9.
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each consists of the amino acid sequence of SEQ ID NO:7;
(b) the first heavy chain polypeptide consists of the amino acid sequence of amino acids 1-453 or 1-454 of any of SEQ ID NOs: 8, 10, and 17-31; and
(c) the second heavy chain polypeptide consists of the amino acid sequence of amino acids 1-453 or 1-454 of SEQ ID NO:9,
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each consists of the amino acid sequence of SEQ ID NO:7;
(b) the first heavy chain polypeptide consists of the amino acid sequence of amino acids 1-453 or 1-454 of SEQ ID NO:8; and
(c) the second heavy chain polypeptide consists of the amino acid sequence of amino acids 1-453 or 1-454 of SEQ ID NO:9,
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In some embodiments, an Abeta-targeting protein comprises a first light chain polypeptide, a second light chain polypeptide, a first heavy chain polypeptide, and a second heavy chain polypeptide wherein:
(a) the first and second light chain polypeptides each consists of the amino acid sequence of SEQ ID NO:7;
(b) the first heavy chain polypeptide consists of the amino acid sequence of amino acids 1-453 or 1-454 of SEQ ID NO: 10; and
(c) the second heavy chain polypeptide consists of the amino acid sequence of amino acids 1-453 or 1-454 of SEQ ID NO:9.
wherein the first light chain polypeptide forms a dimer with the first heavy chain to form an Abeta binding domain, the second light chain polypeptide forms a dimer with the second heavy chain to form an Abeta binding domain, and the first and second Fc polypeptides form an Fc dimer.

In still other embodiments, the Abeta-targeting protein may be fused to a peptide or protein useful in protein purification. Such peptides include, but are not limited to, polyhistidine, epitope tags, (e.g., FLAG, c-Myc, and hemagglutinin tags), glutathione S transferase (GST), thioredoxin, protein A, protein G, and maltose binding protein (MBP). In some cases, the peptide or protein to which the protein is fused may comprise a protease cleavage site, such as a cleavage site for Factor Xa or Thrombin. In certain embodiments, the linkage is cleavable by an enzyme present in the central nervous system.

### III. Nucleic Acids, Vectors, and Host Cells

The Abeta-targeting proteins as described herein can be prepared using recombinant methods. Accordingly, isolated nucleic acids comprising sequences encoding any of the Abeta-targeting proteins described herein or portions thereof are readily generated using methods available in the art. Host cells into which the nucleic acids are introduced and that can be used to replicate the polypeptide-encoding nucleic acids and/or to express the polypeptides are also available in the art. A host cell can be, but is not limited to, a prokaryotic cell or a eukaryotic, the eukaryotic call be, but is not limited to, a yeast cell, an insect cell, or a mammalian cell (e.g., a human cell).

A nucleic acid encoding an Abeta-targeting protein or a portion thereof can be DNA, RNA, cDNA, mRNA, single-stranded, double-stranded, linear or circular.

In some embodiments, a nucleic acid encoding an Abeta-targeting protein comprises a nucleic acid sequence encoding a polypeptide having at least 85% identity, at least 90% identity, or at least 95% identity, to any of SEQ ID NOs: 8, 10, and 17-31, wherein the encoded polypeptide has the amino acids at positions 384, 386, 387, 388, 389, 390, 413, 415, 416, and 421 (according to EU numbering scheme) as shown in Table 1. In some embodiments, the encoding polypeptide further comprises: (a) an alanine at position 234, an alanine at position 235, (b) (a) an alanine at position 234, an alanine at position 235, and a glycine at position 329, or (c) an alanine at position 234, an alanine at position 235, a glycine at position 329, and a tryptophan at position 366.

In some embodiments, a nucleic acid encoding an Abeta-targeting protein comprises a nucleic acid sequence encoding a polypeptide having at least 85% identity, at least 90% identity, or at least 95% identity, to SEQ ID NO:8 or 10, wherein the encoded polypeptide contains: (a) an alanine at position 234, an alanine at position 235, a glycine at position 329, a tryptophan at position 366, a tyrosine at position 384, a threonine at position 386, a glutamate position 387, a tryptophan at position 388, a valine at position 389, a threonine at position 413, a glutamate at position 415, a glutamate at position 416, and a phenylalanine at position 421; or (b) an alanine at position 234, an alanine at position 235, a tryptophan at position 366, a tyrosine at position 384, a threonine at position 386, a glutamate position 387, a tryptophan at position 388, a valine at position 389, a threonine at position 413, a glutamate at position 415, a glutamate at position 416, and a phenylalanine at position 421 (according to EU numbering scheme).

An Abeta-targeting protein may comprise two or more (e.g., three) polypeptides, each of which may be encoded by a separate nucleic sequence. The separate nucleic acid sequence may be present on the same plasmid or vector or different plasmids or vectors. If present on the same plasmid or vector, the separate nucleic acid sequences may be expressed from a single promoter or from different promoters. Method of expressing nucleic acids encoding separate polypeptides from a single promoter are known in the art and include, but are not limited to, the use of 2A elements and internal ribosome entry sites.

A nucleic acid encoding an Abeta-targeting protein or a portion thereof can be provided in a plasmid or vector. The plasmid or vector can be used to replicate the nucleic acid or facilitate expression of the nuclei acid. A plasmid or vector can be, but is not limited to, a viral vector, a phagemid, a yeast chromosomal vector, or a non-episomal mammalian vector.

In some embodiments, the nucleic acid encoding an Abeta-targeting protein or a portion thereof operably linked to one or more regulatory sequences in an expression construct. The expression constructs can be adapted for expression of the polypeptide in a system that production of the Abeta-targeting protein. Such a system can be, but is not limited to, mammalian cell expression system, an insect cell expression system, a yeast cell expression system, or a bacterial cell expression system.

Expression vehicles for production of a recombinant polypeptide include plasmids and other vectors. For instance, suitable vectors include plasmids of the following types: pBR322-derived plasmids, pEMBL-derived plasmids, pEX-derived plasmids, pBTac-derived plasmids, and pETC-derived plasmids for expression in prokaryotic cells, such as E. coli. The pcDNAI/amp, pcDNAEneo, pRc/CMV, pSV2gpt, pSV2neo, pSV2-dhfr, pTk2, pRSVneo, pMSG, pSVT7, pko-neo, and pHyg-derived vectors are examples of mammalian expression vectors suitable for transfection of eukaryotic cells. Alternatively, derivatives of viruses such as the bovine papilloma virus (BPV-1), or Epstein-Barr virus (pHEBo, pREP-derived, and p205) can be used for transient expression of polypeptides in eukaryotic cells. In some embodiments, it may be desirable to express the recombinant polypeptide by the use of a baculovirus expression system. Examples of such baculovirus expression systems include pVL-derived vectors (such as pVL1392, pVL1393, and pVL941), pAcUW-derived vectors (such as pAcUWI), and pBlueBac-derived vectors. Additional expression systems include adenoviral, adeno-associated virus, and other viral expression systems.

A expression vector for expressing an Abeta-targeting protein or a portion thereof, of a plasmid or vector containing the nucleic acid can be transformed, transfected, or transduced into a host cell. The host cell can be, but is not limited to, a mammalian cell, a yeast cell, an insect cell, prokaryotic cell, Chinese hamster ovary (CHO) cell, a baby hamster kidney (BHK) cell, a NSO cell, a YO cell, a HEK293 cell, a COS cell, a Vero cell, or a HeLa cell. The host cell containing the expression vector can be cultured under appropriate conditions to allow expression of the Abeta-targeting protein or a portion thereof.

An Abeta-targeting protein can be manufactured by culturing a host cell comprising one or more nucleic acids encoding the Abeta-targeting protein, expressing the Abeta-targeting protein, and isolating the expressed Abeta-targeting protein from the culture. In some embodiments, the host cell comprises: (a) a first nucleic acid encoding a polypeptide having at least 85% identity, at least 90% identity, at least 95% identity, or 100% identify to any of SEQ ID NOs: 8, 10, or 17-31; (b) a second nucleic acid encoding a polypeptide having at least 85% identity, at least 90% identity, at least 95% identity, or 100% identify to SEQ ID NO: 9; and (c) a third nucleic acid encoding a polypeptide having at least 85% identity, at least 90% identity, at least 95% identity, or 100% identify to SEQ ID NO:7

### IV. Formulations

Any of the described Abeta-targeting proteins can be prepared, provided, or formulated as a salt, mixed salt, or a free-acid. Suitable pharmaceutically acceptable salts include, but are not limited to, sodium, potassium, calcium, and magnesium salts.

Any of the described Abeta-targeting proteins can be provided in or formulated in a pharmaceutical composition. A pharmaceutical composition or medicament includes a pharmaceutically effective amount of at least one of the described Abeta-targeting proteins and optionally one or more pharmaceutically acceptable excipients. Pharmaceutically acceptable excipients (excipients) are substances other than the Active Pharmaceutical ingredient (API, therapeutic product (e.g., Abeta-targeting protein)) that are intentionally included in the pharmaceutical composition. Excipients do not exert or are not intended to exert a therapeutic effect at the intended dosage. Excipients may act to (a) aid in processing of the API during manufacture, (b) protect, support or enhance stability, bioavailability, or patient acceptability of the API, (c) assist in product identification, and/or (d) enhance any other attribute of the overall safety, effectiveness, or delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The carrier can be, but is not limited to, a solvent or dispersion medium containing, for example, water, saline, phosphate buffered saline, Ringer's solution, ethanol, polyol (for example, glycerol, propylene glycol, and liquid polyethylene glycol), and suitable mixtures thereof. A carrier may also contain adjuvants or additives such as preservatives, wetting agents, emulsifying agents and dispersing agents. A carrier may also contain isotonic agents, such as sugars, polyalcohols, sodium chloride, and the like.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, the pharmaceutical compositions further comprise one or more additional active ingredients. The additional active pharmaceutical ingredient can be, but is not limited to, a small molecule drug.

An Abeta-targeting protein or pharmaceutical composition containing an Abeta-targeting protein can be formulated as a liquid formulation or as a solid formulation (including a powder or lyophilized formulation, e.g., a lyophilized cake or powder).

In some embodiments, the pharmaceutical compositions described herein can be formulated for administration to a subject.

As disclosed above, an Abeta-targeting protein or pharmaceutical composition containing an Abeta-targeting protein can be formulated for parenteral administration by injection, e.g., by bolus injection or continuous infusion. For injection, the Abeta-targeting protein can be formulated into preparations by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers, and preservatives. In some embodiments, polypeptides can be formulated in aqueous solutions, preferably in physiologically compatible buffers such as Hanks' solution, Ringer's solution, or physiological saline buffer. Formulations for injection can be presented in unit dosage form, e.g., in ampules or in multi-dose containers, with an added preservative. The compositions can take such forms as suspensions, solutions, or emulsions in oily or aqueous vehicles, and can contain formulatory agents such as suspending, stabilizing, and/or dispersing agents.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the invention may vary depending on the particular use envisioned. The determination of the appropriate dosage or route of administration is well within the skill of one in the art.

### A. Kits

In some embodiments, kits comprising an Abeta-targeting protein as described herein are provided. In some embodiments, the kits are for use in preventing or treating a neurological disorder associated with Abeta.

The described Abeta-targeting proteins and pharmaceutical compositions comprising Abeta-targeting proteins disclosed herein may be packaged or included in a kit, container, pack, or dispenser. The Abeta-targeting proteins and pharmaceutical compositions comprising the Abeta-targeting proteins may be packaged in pre-filled syringes or vials. Any of the Abeta-targeting proteins or pharmaceutical compositions containing Abeta-targeting proteins described herein can be formulated or packaged in single-dose or multi-dose format. Any of the Abeta-targeting proteins or pharmaceutical compositions containing the Abeta-targeting proteins described identified herein can be formulated for repeat dosing.

In some embodiments, the kit further comprises one or more additional therapeutic agents. For example, in some embodiments, the kit comprises a transferrin receptor-binding polypeptide as described herein and further comprises one or more additional therapeutic agents for use in the treatment of a neurological disorder.

The kit or package may further contain instructions for use. Instructions include documents describing relevant materials or methodologies pertaining to the kit. The instructions may include one or more of: background information, list of components and their availability information (purchase information, etc.), brief or detailed protocols for using the kit, trouble-shooting guidance, references, technical support, indications, usage, dosage, administration, contraindications, and/or warnings concerning the use of the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration.

### V. Therapeutic Methods

The described Abeta-targeting proteins may be used deliver a compound having a Abeta-binding and effector function capability into a brain of a subject, i.e., across the blood-brain barrier.

The described Abeta-targeting proteins can be administered to a subject to treat a disease or condition mediated at least in part by amyloid beta or amyloid beta plaques. After administration to a subject, the described Abeta-targeting proteins are translocated across the BBB to the brain where they bind to amyloid plaques, cerebrovascular Abeta, or diffuse Abeta deposits. Binding of the Abeta-tarting proteins to amyloid plaques, cerebrovascular Abeta, or diffuse Abeta can facilitate an immune response, such as ADCP, against the amyloid plaques, cerebrovascular Abeta, or diffuse Abeta.

Described are methods of treating a subject suffering from a disease or condition mediated at least in part by amyloid beta or amyloid beta plaques, comprising administering to the subject an Abeta-targeting protein or a composition containing an Abeta-targeting protein. In some embodiments the disease or condition mediated at least in part by amyloid beta or amyloid beta plaques comprises a neurodegenerative diseases or condition. The neurodegenerative disease can be, but is not limited to, Alzheimer's disease.

Described are methods of reducing amyloid plaques in a brain of a subject, comprising administering to the subject an Abeta-targeting protein or a composition containing an Abeta-targeting protein. Reducing amyloid plaques can comprise increasing Abeta phagocytosis and/or increasing recruitment of microglia to Abeta-positive plaques in the subject. In some embodiments, the subject has or is diagnosed with a neurodegenerative disease or is at increased risk of developing a neurodegenerative disease. In some embodiments, the neurodegenerative disease is Alzheimer's disease.

Described are methods of reducing amyloid plaques in a brain of a subject, comprising administering to the subject an Abeta-targeting protein or a composition containing an Abeta-targeting protein. In some embodiments, the subject has or is diagnosed with a neurodegenerative disease or is at increased risk of developing a neurodegenerative disease. In some embodiments, the neurodegenerative disease is Alzheimer's disease.

Described are methods of treating cognitive impairment, memory loss, and/or dementia, in a subject comprising administering to the subject an Abeta-targeting protein or a composition containing an Abeta-targeting protein. In some embodiments, the subject has or is diagnosed with a neurodegenerative disease or is at increased risk of developing a neurodegenerative disease. In some embodiments, the neurodegenerative disease is Alzheimer's disease.

Described are methods of treating loss of neuronal connections in the brain of a subject comprising administering to the subject an Abeta-targeting protein or a composition containing an Abeta-targeting protein. In some embodiments, the subject has or is diagnosed with a neurodegenerative disease or is at increased risk of developing a neurodegenerative disease. In some embodiments, the neurodegenerative disease is Alzheimer's disease.

Described are methods of delaying or preventing one or more symptoms or pathological conditions associated at least in part with accumulation of amyloid plaques in the brain of a subject comprising administering to the subject an Abeta-targeting protein or a composition containing an Abeta-targeting protein. In some embodiments, the subject has or is diagnosed with a neurodegenerative disease or is at increased risk of developing a neurodegenerative disease. In some embodiments, the neurodegenerative disease is Alzheimer's disease.

Described are methods of reducing amyloid-related imaging abnormalities (ARIA) in a subject comprising administering to the subject an Abeta-targeting protein or a composition containing an Abeta-targeting protein. In some embodiments, the subject has or is diagnosed with a neurodegenerative disease or is at increased risk of developing a neurodegenerative disease. In some embodiments, the neurodegenerative disease is Alzheimer's disease. In some embodiments the subject is in need of receiving, or is scheduled to receive, a treatment for the neurodegenerative disease, e.g., Alzheimer's disease. In some embodiments, ARIA is associated with the treatment of the neurodegenerative disease, e.g., Alzheimer's disease. In some embodiments, the treatment for the neurodegenerative disease, e.g., Alzheimer's disease, is a therapeutic antibody.

In some embodiments, ARIA includes two classes of MRI signal abnormalities: ARIA-E (oedema/effusion) and ARIA-H (haemosiderosis/microhaemorrhages). ARIA-E refers to the extravasation of proteinaceous fluid resulting in interstitial vasogenic oedema or sulcal effusion in the leptomeningeal/subpial space. These manifest as hyperintense parenchymal or sulcal abnormalities on T2-weighted and fluid-attenuated inversion recovery (FLAIR) sequence images. ARIA-H refers to microhaemorrhages (mH) or macrohaemorrhages observed as hypointense haemosiderin deposition. These reflect iron accumulation following the breakdown of extravasated haemoglobin on gradient recalled echo (GRE)/T2* images or with enhanced visualization processing by susceptibility weighting imaging (SWI) sequences. In some embodiments, the methods reduce the incidence of ARIA-E events. In some embodiments, the methods reduce the incidence of ARIA-H events. In other embodiments, the methods reduce the incidence of both ARIA-E and ARIA-H events.

The Abeta-targeting protein can be administered parenterally, intravenously, or intrathecally, by epidural administration, or intracerebroventricularly. Administration can be by bolus injection, infusion, or a combination thereof. Infusion can occur over a period of from about 10 to about 30 minutes, or over a period of at least 1 hour to about 4 hours or more.

The Abeta-targeting protein can be administered in combination with one or more additional therapies. The one or more additional therapies can include, but is not limited to, an agent useful for treating Alzheimer's disease.

### A. Alzheimer's Disease

“Alzheimer's disease” (AD) is a dementia that is primarily identified by clinical diagnosis and established by markers of the disease. AD is a continuum having certain operationally defined stages of disease progression. AD pathology begins prior to the onset of clinical symptoms. Amyloid plaques, one marker of AD pathology, can form 10-20 years prior to the onset of AD-associated dementia. The currently recognized stages of AD include preclinical, prodromal, mild, moderate, and severe, although the stages are neither discrete nor distinct. Nevertheless, AD stage and progression can be monitored by measuring or assessing amyloid beta accumulation (CSF/PET), synaptic dysfunction (FDG-PET/fMRI), tau-mediated neuronal injury (CSF), brain structure (volumetric MRI), cognition, and clinical function. Systems for diagnosing and monitoring AD include, but are not limited to: International Working Group (IWG) new research criteria for diagnosis of AD (Dubois B et al. Lancet Neurol 2007; 6 (8): 734-736), IWG research criteria, (Dubois et al. Lancet Neurol 2010; 9 (11): 1118-27), NIA/AA Criteria (Jack C R et al. Alzheimer's Dement 2011; 7 (3): 257-62), and DSM-5 criteria (American Psychiatric Association, DSM-5, 2013).

The described Abeta-targeting proteins can be administered to a subject to treat a disease or condition associated with amyloid beta, or to reduce amyloid beta plaques in the brain of the subject. In some embodiments, treating a disease or condition associated with amyloid beta comprises: (a) alleviating or ameliorating the amyloid beta-associated disease; (b) alleviating or ameliorating one or more symptoms or pathological conditions associated the amyloid beta-associated disease; or (c) reducing amyloid beta plaques in the brain of the subject; or (d) preventing or delaying at least one symptom or pathological condition in a subject having or at risk of developing an amyloid beta-associated disease, or (e) prolonging survival of a subject diagnosed with the amyloid beta-associated disease. In some embodiments, treating a disease or condition associated with amyloid beta comprises reducing amyloid-related imaging abnormalities (AIRA) in the subject. In some embodiments, the disease or condition associated with amyloid beta (amyloid beta-associated disease) is a neurodegenerative disease. In some embodiments, the neurodegenerative disease is AD. Pathological conditions associated the amyloid beta-associated disease include, but are not limited to: cognitive impairment, memory loss, dementia, or loss of neuronal connections in the brain.

Described are methods of treating a subject (e.g., a human subject) having a pathological condition or at risk of developing a pathological condition mediated at least in part by amyloid beta, the methods comprising administering to the subject a effective amount (e.g., a therapeutically effective amount or a prophylactically effective amount) of an Abeta-targeting protein or a composition containing the Abeta-targeting protein. Administering the Abeta-targeting protein or composition containing the Abeta-targeting protein to a subject can optionally be combined with one or more steps of administering one or more additional (i.e., second, third, etc.) therapeutics or treatments.

In some embodiments, the method further comprises administering to the subject one or more additional therapeutic agents. For example, in some embodiments for treating a disease of the brain or central nervous system, the method may comprise administering to the subject a neuroprotective agent, e.g., an anticholinergic agent, a dopaminergic agent, a glutamatergic agent, a histone deacetylase (HDAC) inhibitor, a cannabinoid, a caspase inhibitor, melatonin, an anti-inflammatory agent, a hormone (e.g., estrogen or progesterone), or a vitamin. In some embodiments, the method comprises administering to the subject an agent for use in treating a cognitive or behavioral symptom of a neurological disorder (e.g., an antidepressant, a dopamine agonist, or an anti-psychotic).

VL Sequences

| | | SEQ ID |
|---|---|---|
| Description | Sequence | NO: |
| VHCDR1 | SYGMH | 1 |
| VHCDR2 | VIWFDGTKKYYTDSVKG | 2 |
| VHCDR3 | DRGIGARRGPYYMDV | 3 |
| VLCDR1 | RASQSISSYLN | 4 |
| VLCDR2 | AASSLQS | 5 |
| VLCDR3 | QQSYSTPLT | 6 |
| anti-Abeta | DIQMTQSPSSLSASVGDRVTITCRASQSISSYLNWYQQKPGKAPKLLIYAASSLQSGVP | 7 |
| light chain | SRFSGSGSGTDFTLTISSLQPEDFATYYCQQSYSTPLTFGGGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSFNRGEC | |
| ATV35.23. | QVQLVESGGGVVQPGRSLRLSCAASGFAFSSYGMHWVRQAPGKGLEWVAVIWEDGTKKY | 8 |
| 3^{cisLALAPG}: | YTDSVKGRFTISRDNSKNTLYLQMNTLRAEDTAVYYCARDRGIGARRGPYYMDVWGKGT | |
| Abeta | TVTVSSASTKGPSVFPLAPSSKSTSGGTAALGCLVKDYFPEPVTVSWNSGALTSGVHTF | |
| TfR- | PAVLQSSGLYSLSSVVTVPSSSLGTQTYICNVNHKPSNTKVDKKVEPKSCDKTHTCPPC | |
| binding | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALGAPIEKTISKAKGQPREPQ | |
| | VYTLPPSRDELTKNQVSLWCLVKGFYPSDIAVEWESYGTEWVNYKTTPPVLDSDGSFEL | |
| | YSKLTVTKEEWQQGFVFSCSVMHEALHNHYTQKSLSLSPGK | |
| ATV35.23. | QVQLVESGGGVVQPGRSLRLSCAASGFAFSSYGMHWVRQAPGKGLEWVAVIWEDGTKKY | 9 |
| 3:Abeta | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPELLGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLSCAVKGFYPSDIAVEWESNGQPENNYKTTPPVLDSDGSFFL | |
| | VSKLTVDKSRWQQGNVFSCSVMHEALHNHYTQKSLSLSPGK | |
| ATV35.23. | QVQLVESGGGVVQPGRSLRLSCAASGFAFSSYGMHWVRQAPGKGLEWVAVIWEDGTKKY | 10 |
| 3^{cisLALA}: | YTDSVKGRFTISRDNSKNTLYLQMNTLRAEDTAVYYCARDRGIGARRGPYYMDVWGKGT | |
| Abeta | TVTVSSASTKGPSVFPLAPSSKSTSGGTAALGCLVKDYFPEPVTVSWNSGALTSGVHTE | |
| TfR- | PAVLQSSGLYSLSSVVTVPSSSLGTQTYICNVNHKPSNTKVDKKVEPKSCDKTHTCPPC | |
| binding | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWVNYKTTPPVLDSDGSFFL | |
| | YSKLTVTKEEWQQGFVFSCSVMHEALHNHYTQKSLSLSPGK | |
| TfR | MMDQARSAFSNLEGGEPLSYTRESLARQVDGDNSHVEMKLAVDEEENADNNTKANVTKP | 15 |
| | KRCSGSICYGTIAVIVEFLIGEMIGYLGYCKGVEPKTECERLAGTESPVREEPGEDEPA | |
| | ARRLYWDDLKRKLSEKLDSTDFTGTIKLLNENSYVPREAGSQKDENLALYVENQFREFK | |
| | LSKVWRDQHFVKIQVKDSAQNSVIIVDKNGRLVYLVENPGGYVAYSKAATVTGKLVHAN | |
| | FGTKKDFEDLYTPVNGSIVIVRAGKITFAEKVANAESINAIGVLIYMDQTKEPIVNAEL | |
| | SFFGHAHLGTGDPYTPGFPSENHTQFPPSRSSGLPNIPVQTISRAAAEKLEGNMEGDCP | |
| | SDWKTDSTCRMVTSESKNVKLTVSNVLKEIKILNIFGVIKGFVEPDHYVVVGAQRDAWG | |
| | PGAAKSGVGTALLLKLAQMFSDMVLKDGFQPSRSIIFASWSAGDEGSVGATEWLEGYLS | |
| | SLHLKAFTYINLDKAVIGTSNEKVSASPLLYTLIEKTMQNVKHPVTGQFLYQDSNWASK | |
| | VEKLTLDNAAFPFLAYSGIPAVSFCFCEDTDYPYLGTTMDTYKELIERIPELNKVARAA | |
| | AEVAGQFVIKLTHDVELNLDYERYNSQLLSFVRDLNQYRADIKEMGLSLQWLYSARGDE | |
| | FRATSRLTTDEGNAEKTDRFVMKKLNDRVMRVEYHFLSPYVSPKESPFRHVFWGSGSHT | |
| | LPALLENLKLRKQNNGAFNETLERNQLALATWTIQGAANALSGDVWDIDNEF | |
| TfR apical | NSVIIVDKNGRLVYLVENPGGYVAYSKAATVTGKLVHANFGTKKDFEDLYTPVNGSIVI | 16 |
| domain | VRAGKITFAEKVANAESLNAIGVLIYMDQTKFPIVNAELSFFGHAHLGTGDPYTPGFPS | |
| | FNHTQFPPSRSSGLPNIPVQTISRAAAEKLFGNMEGDCPSDWKTDSTCRMVTSESKNVK | |
| | LTVS | |
| 35.d1.1 | QVQLVESGGGVVQPGRSLRLSCAASGFAFSSYGMHWVRQAPGKGLEWVAVIWEDGTKKY | 17 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGNEWSSYKTTPPVLDSDGSFFL | |
| | YSKLTVTKSEWQQGFVFSCSVMHEALHNHYTQKSLSLSPGK | |
| 35.d1.2 | QVQLVESGGGVVQPGRSLRLSCAASGFAFSSYGMHWVRQAPGKGLEWVAVIWEDGTKKY | 18 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWTSYKTTPPVLDSDGSFFL | |
| | YSKLTVTKSEWQQGFVFSCSVMHEALHNHYTQKSLSLSPGK | |
| 35.d1.3 | QVQLVESGGGVVQPGRSLRLSCAASGFAFSSYGMHWVRQAPGKGLEWVAVIWEDGTKKY | 19 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWSSYKTTPPVLDSDGSFFL | |
| | YSKLTVTKSEWQQGYVFSCSVMHEALHNHYTQKSLSLSPGK | |
| 35.d1.4 | QVQLVESGGGVVQPGRSLRLSCAASGFAFSSYGMHWVRQAPGKGLEWVAVIWEDGTKKY | 20 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESFGTEWSSYKTTPPVLDSDGSFFL | |
| | YSKLTVSKSEWQQGFVFSCSVMHEALHNHYTQKSLSLSPGK | |
| 35.d1.7 | QVQLVESGGGVVQPGRSLRLSCAASGFAFSSYGMHWVRQAPGKGLEWVAVIWEDGTKKY | 21 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWSSYKTTPPVLDSDGSFFL | |
| | YSKLTVSKSEWQQGYVFSCSVMHEALHNHYTQKSLSLSPGK | |
| 35.d1.9 | QVQLVESGGGVVQPGRSLRLSCAASGFAFSSYGMHWVRQAPGKGLEWVAVIWEDGTKKY | 22 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWTNYKTTPPVLDSDGSFFL | |
| | YSKLTVTKSEWQQGFVFSCSVMHEALHNHYTQKSLSLSPGK | |
| 35.d1.10 | QVQLVESGGGVVQPGRSLRLSCAASGFAFSSYGMHWVRQAPGKGLEWVAVIWEDGTKKY | 23 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWVNYKTTPPVLDSDGSFEL | |
| | YSKLTVTKSEWQQGFVFSCSVMHEALHNHYTQKSLSLSPGK | |
| 35.d1.11 | QVQLVESGGGVVQPGRSLRLSCAASGFAFSSYGMHWVRQAPGKGLEWVAVIWEDGTKKY | 24 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWSNYKTTPPVLDSDGSFFL | |
| | YSKLTVTKSEWQQGYVFSCSVMHEALHNHYTQKSLSLSPGK | |
| 35.d1.12 | QVQLVESGGGVVQPGRSLRLSCAASGFAFSSYGMHWVRQAPGKGLEWVAVIWFDGTKKY | 25 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESFGTEWSNYKTTPPVLDSDGSFFL | |
| | YSKLTVSKSEWQQGFVFSCSVMHEALHNHYTQKSLSLSPGK | |
| 35.d1.15 | QVQLVESGGGVVQPGRSLRLSCAASGFAFSSYGMHWVRQAPGKGLEWVAVIWFDGTKKY | 26 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWSNYKTTPPVLDSDGSFEL | |
| | YSKLTVSKSEWQQGYVFSCSVMHEALHNHYTQKSLSLSPGK | |
| 35.d1.18 | QVQLVESGGGVVQPGRSLRLSCAASGFAFSSYGMHWVRQAPGKGLEWVAVIWEDGTKKY | 27 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESFGTEWSNYKTTPPVLDSDGSFEL | |
| | YSKLTVSKSEWQQGYVFSCSVMHEALHNHYTHKSLSLSPGK | |
| 35.d1.20 | QVQLVESGGGVVQPGRSLRLSCAASGFAFSSYGMHWVRQAPGKGLEWVAVIWEDGTKKY | 28 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWVNYKTTPPVLDSDGSFFL | |
| | YSKLTVSKSEWQQGFVFSCSVMHEALHNHYTQKSLSLSPGK | |
| 35.d1.22 | QVQLVESGGGVVQPGRSLRLSCAASGFAFSSYGMHWVRQAPGKGLEWVAVIWEDGTKKY | 29 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGNEWSNYKTTPPVLDSDGSFFL | |
| | YSKLTVSKEEWQQGFVFSCSVMHEALHNHYTQKSLSLSPGK | |
| 35.d1.23 | QVQLVESGGGVVQPGRSLRLSCAASGFAFSSYGMHWVRQAPGKGLEWVAVIWFDGTKKY | 30 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWTNYKTTPPVLDSDGSFEL | |
| | YSKLTVSKEEWQQGFVFSCSVMHEALHNHYTQKSLSLSPGK | |
| 35.d1.24 | QVQLVESGGGVVQPGRSLRLSCAASGFAFSSYGMHWVRQAPGKGLEWVAVIWEDGTKKY | 31 |
| | YTDSVKGRFTISRDNSKNTLYLQMNTLRAEDTAVYYCARDRGIGARRGPYYMDVWGKGT | |
| | TVTVSSASTKGPSVFPLAPSSKSTSGGTAALGCLVKDYFPEPVTVSWNSGALTSGVHTF | |
| | PAVLQSSGLYSLSSVVTVPSSSLGTQTYICNVNHKPSNTKVDKKVEPKSCDKTHTCPPC | |
| | PAPEAAGGPSVFLFPPKPKDTLMISRTPEVTCVVVDVSHEDPEVKFNWYVDGVEVHNAK | |
| | TKPREEQYNSTYRVVSVLTVLHQDWLNGKEYKCKVSNKALPAPIEKTISKAKGQPREPQ | |
| | VYTLPPSRDELTKNQVSLWCLVKGFYPSDIAVEWESYGTEWSNYKTTPPVLDSDGSFEL | |
| | YSKLTVSKEEWQQGYVFSCSVMHEALHNHYTQKSLSLSPGK | |

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Abeta-Targeting Proteins Bind to Oligomeric and Fibrillar Abeta, but not Monomeric Abeta

For preparation of monomeric Abeta peptides, Abeta₁₋₄₀ powder (Anaspec, AS-24235) was reconstituted in hexafluoroisopropanol (HFIP) at a concentration of 1 mg/mL. The Abeta₁₋₄₀ was aliquoted into 50 μL/vial, each aliquot was dried under N₂ gas, and stored as Abeta₁₋₄₀ film in HFIP at −80° C. To form the monomers, the Abeta₁₋₄₀ film HFIP was dissolved in dimethyl sulfoxide (DMSO), and then diluted to the target concentration in assay buffer before using.

For preparation of oligomeric Abeta₁₋₄₂ and fibrillar Abeta₁₋₄₂, Abeta₁₋₄₂ Film in HFIP (Anaspec, AS-64129) was resuspended in DMSO at concentration of 5 mg/mL, and then diluted into PBS at a concentration of 1 mg/mL. To form the oligomeric Abeta₁₋₄₂, diluted Abeta₁₋₄₂ in PBS was incubated for 3 days at 37° C. To form the fibrillar Abeta₁₋₄₂, diluted Abeta₁₋₄₂ in PBS was incubated for 7 days at 37° C. and then was centrifuged at 14,000×g for 15 min at 4° C. Fibrillar Abeta₁₋₄₂ was dissolved in PBS. To validate quality of Abeta preparation, dynamic light scattering (DLS) was used.

The binding specificity of ATVs to Abeta was measured by ELISA (IBL, 27725). Four different ATVs were analyzed.
1. ATV35.23.4^{cisLALA}: having anti-Abeta IgG Abeta Fab binding domains and an Fc dimer comprising a first Fc polypeptide having a TfR-binding region (affinity for TfR of about 600 nM) and L324A and L235A substitutions to reduce FcγR binding (also termed ATV35.23.4^{cisLALA}:Abeta).
2. ATV35.23.4^{cisLALAPG}: having anti-Abeta IgG Abeta Fab binding domains and an Fc dimer comprising a first Fc polypeptide having a TfR-binding region (affinity for TfR of about 600 nM) and L324A, L235A, and P329G substitutions to reduce FcγR binding (also termed ATV35.23.4^{cisLALAPG}:Abeta).
3. ATV35.23.3^{cisLALA} (Abeta-targeting protein): having anti-Abeta IgG Abeta Fab binding domains and an Fc dimer comprising a first Fc polypeptide having a TfR-binding region (affinity for TfR of about 1100 nM) and L324A and L235A substitutions to reduce FcγR binding (also termed ATV35.23.3^{cisLALA}:Abeta).
4. ATV35.23.3^{cisLALAPG} (Abeta-targeting protein): having anti-Abeta IgG Abeta Fab binding domains and an Fc dimer comprising a first Fc polypeptide having a TfR-binding region (affinity for TfR of about 600 nM) and L324A, L235A, and P329G substitutions to reduce FcγR binding (also termed ATV35.23.3^{cisLALAPG}:Abeta).

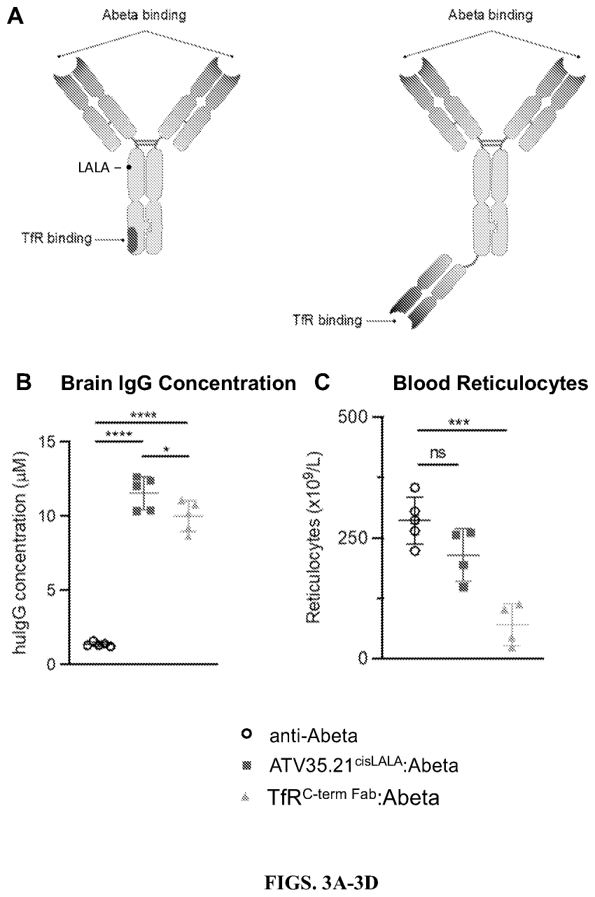

The ATVs had a configuration as shown in FIG. 3A (left diagram).

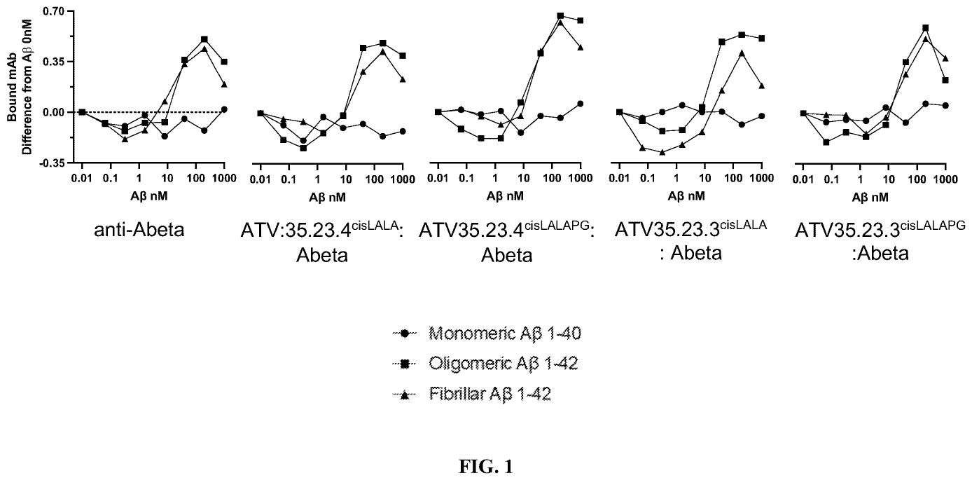

An anti-Abeta IgG antibody was used as a control. The Abeta-targeting proteins contained the antibody binding domains of the anti-Abeta IgG antibody. 20 μL of 0.4 nM antibodies and 100 μL of monomeric, oligomeric or fibrillar Abeta were mixed and incubated for 60 min at 4° C. 100 μl of each solution was added into precoated plate for 60 min at 4° C. After three washes with PBS containing 0.05% Tween-20 (PBST), each well was treated with 100 μL of horseradish peroxidase-conjugated goat anti-Human for 1 h at room temperature. After three washes with PBST, each well was added 100 μL of TMB, chromogen and then added 100 μL of stop solution. ELISA signal was measured with a microplate luminometer (BioTek Neo2). Results (FIG. 1) indicate that all anti-Abeta antibodies tested specifically bound to oligomeric Abeta₁₋₄₂ and fibrillar Abeta₁₋₄₂ to a comparable degree, and did not bind to monomeric Abeta₁₋₄₀.

### Example 2. The Weaker Affinity ATV35.23.3^{cisLALA}:Abeta Molecule has Higher Brain Exposure than ATV35.23.4^{cisLALA}:Abeta

TfR^{mu/hu} KI mice received a single 25 mg/kg IV dose of anti-Abeta or Abeta-targeting protein (ATV35.23.3 and ATV35.23.4) (n=4-5/group). Blood was collected at 30 minutes and terminal blood and fresh brain (following perfusion) samples were collected and snap frozen at 1, 2, 4, and 7 days post-dose to evaluate huIgG concentrations in plasma and brain lysate. Whole blood was collected on days 1, 2, and 4 for measurement of reticulocytes.

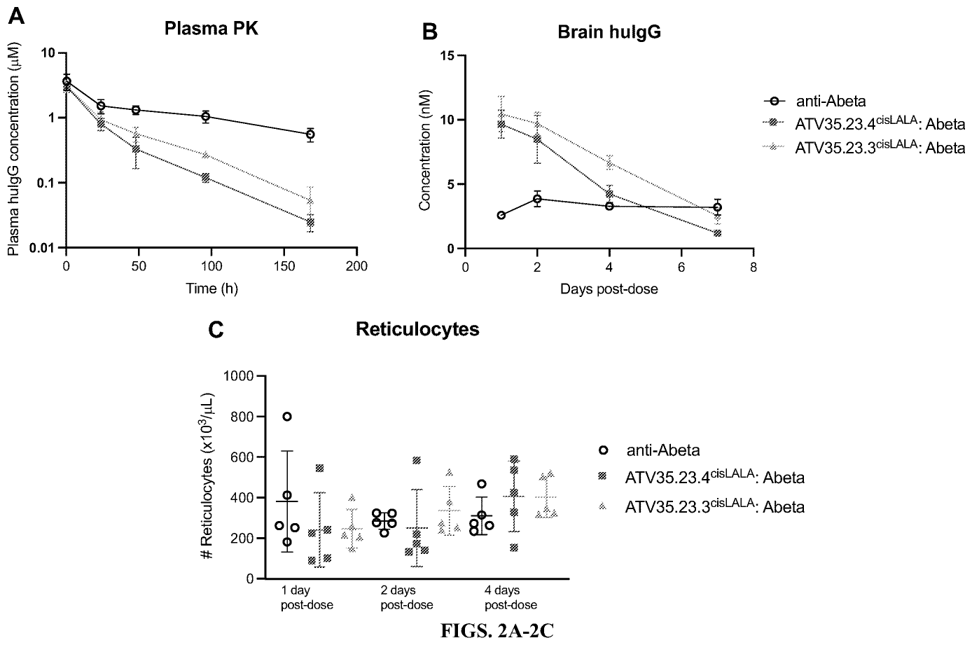

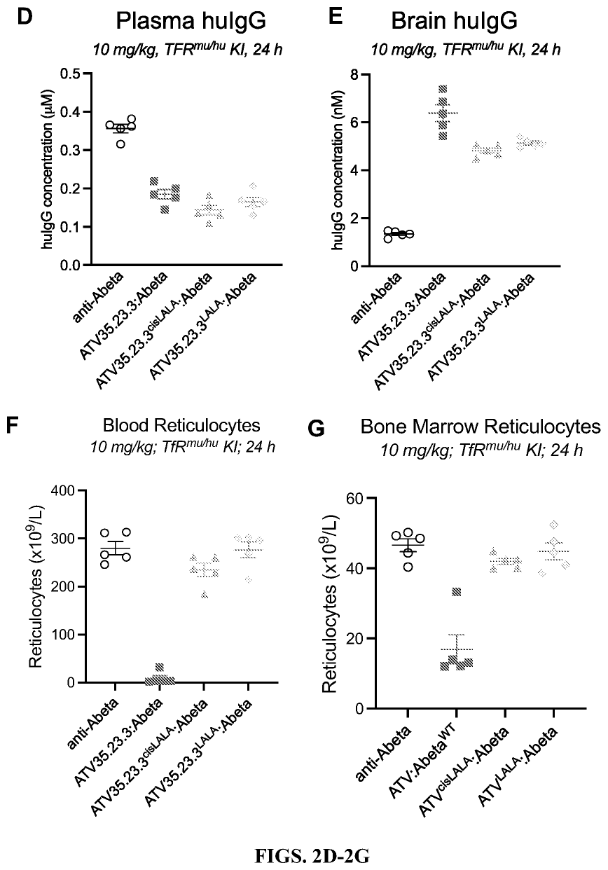

The plasma PK profile showed affinity-dependent, TfR-mediated clearance resulting in faster clearance of ATV molecules from the plasma following a single IV dose of anti-Abeta or ATV:Abeta molecules in TfR^{mu/hu} KI mice (FIG. 2A). The brain PK profile indicated approximately 4-fold higher brain concentrations for ATV:Abeta molecules at 1 day post-dose and significantly higher levels 2 days and 4 days (ATV35.23.3 only) post-dose (FIG. 2B) (two-way ANOVA, Dunnett's post hoc). Surprisingly, the weaker TfR affinity ATV:Abeta molecule (ATV35.23.3^{cisLALA}:Abeta) showed higher Cmax brain concentrations than the stronger TfR affinity variant ATV35.23.4^{cisLALA}:Abeta, which does not align with the expected relationship between TfR affinity and brain uptake. Circulating reticulocytes in the blood of ATV-treated mice showed no differences from the anti-Abeta group at any time point (FIG. 2C) (one-way ANOVA, Dunnett's post hoc), suggesting that cisLALA protects against effector function-mediated reticulocyte loss.

Additionally, to demonstrate the impact of the LALA mutations, TfR^{mu/hu} KI mice received a single 10 mg/kg IV dose of anti-Abeta or ATV:Abeta molecules (n=5/group).

For this experiment, three Abeta-targeting proteins were compared against the anti-Abeta IgG antibody.
1. ATV35.23.3: having anti-Abeta IgG Abeta Fab binding domains and an Fc dimer comprising a first Fc polypeptide having a TfR-binding region (affinity for TfR of about 1100 nM) (also termed ATV35.23.3:Abeta).
2. ATV35.23.3^{cisLALA} (Abeta-targeting protein): having anti-Abeta IgG Abeta Fab binding domains and an Fc dimer comprising a first Fc polypeptide having a TfR-binding region (affinity for TfR of about 1100 nM) and L324A and L235A substitutions to reduce FcγR binding (also termed ATV35.23.3^{cisLALA}. Abeta).
3. ATV35.23.3^{LALA} (Abeta-targeting protein): having anti-Abeta IgG Abeta Fab binding domains and an Fc dimer comprising a first Fc polypeptide having a TfR-binding region (affinity for TfR of about 1100 nM) and L324A and L235A substitutions to reduce FcγR binding and a second Fc polypeptide having L324A and L235A substitutions to reduce FcγR binding (also termed ATV35.23.3^{LALA}:Abeta).

Terminal blood and fresh brain (following perfusion) were collected and snap frozen 24 h post-dose to evaluate huIgG concentrations in plasma and brain lysate. Fresh bone marrow from femurs was collected and stained for Ter119 (erythroid lineage) and CD44 and the population of reticulocytes determined using flow cytometry. Terminal whole blood was also collected for measurement of reticulocytes.

As expected, the terminal plasma demonstrated all ATVs had similar exposure and lower huIgG concentrations compared to anti-Abeta (FIG. 2D), while brain showed higher concentrations of ATVs compared to anti-Abeta (FIG. 2E). Blood reticulocytes demonstrated a clear loss following treatment with the effector-positive ATV35.23.3:Abeta which had a WT Fc region compared to anti-Abeta, but a single cisLALA mutation (ATV35.23.3^{cisLALA}:Abeta) or two LALA mutations (ATV: 35.23.3^{LALA}:Abeta) completely prevented the loss of reticulocytes (FIG. 2F). Similarly, bone marrow reticulocytes only showed a loss of cells for the effector positive ATV35.23.3:Abeta which had a WT Fc (FIG. 2G) and suggests that this architecture enables TfR-mediated cell killing of reticulocytes, while cisLALA and LALA mutations appear to completely prevent this.

### Example 3. Brain Uptake and Blood Reticulocyte Comparison of ATV and C-Terminal TfR Fab Formats

TfR^{mu/hu} KI mice received a single 10 mg/kg IV dose of anti-Abeta, and ATV:Abeta (FIG. 3A left diagram) or an anti-Abeta TfR Fab fusion molecule (FIG. 3A, right diagram) (n=5/group). This right diagram molecule was a fusion of an anti-Abeta IgG with a WT Fc and a Fab fragment that binds to TfR. The affinities of the two molecules (left and right diagrams) for TfR were similar. Terminal blood and fresh brain (following perfusion) were collected and snap frozen 24 h post-dose to evaluate circulating reticulocyte numbers in whole blood and brain huIgG concentrations. The TfR-targeting molecules showed similar brain concentrations as would be expected for matched TfR affinities (FIG. 3B). However, circulating reticulocytes showed significant reduction for the anti-Abeta TfR Fab fusion molecule (one-way ANOVA, Dunnett's post hoc) but not for ATV:Abeta (FIG. 3C), suggesting cisLALA better protects against effector function-mediated reticulocyte loss and has improved hematology safety profile compared to the anti-Abeta TfR Fab (TfR^{C-term Fab}:Abeta) fusion molecule.

### Example 4. PK Profile and Plaque Microglia Recruitment in App^{SAA} KI TfR^{mu/Hu} KI Mice

ATV35.23.3^{cisLALA}:Abeta exhibited improved brain exposure, plaque immunodecoration, and plaque reduction after a single dose compared to ATV35.23.4^{cisLALA}:Abeta.

App^{SAA} KI; TfR^{mu/hu} KI mice received a single 10 mg/kg IV dose of anti-Abeta or ATV:Abeta molecules (n=4-5/group). Terminal blood and fresh brain (following perfusion) were collected and snap frozen at 2, 7, and 14 days post-dose to evaluate huIgG concentrations in plasma and brain lysate. One fresh brain hemisphere per animal was immersion fixed for approximately 24 h at 4° C. for immunohistochemistry. Three to four replicate sagittal brain sections (40 μm) were selected for each animal and stained by incubation in blocking buffer, incubation overnight in primary/secondary antibodies (CD68, Biorad, MCA1957; Abeta, IBL America 18584; and donkey-anti-huIgG, Jackson, 709-606-149) at 4° C., followed by washes and incubation with secondary antibodies (donkey-anti-rat, Invitrogen, SA5-10027; donkey-anti-rabbit, Invitrogen, A21206) and DAPI (5 μg/mL, Invitrogen, D1306), before washes and coverslipping with Prolong Glass (Invitrogen, P36984). Slides were imaged using a Zeiss Axioscan.Z1 slide scanner at 20× magnification and processed using custom macros in Zeiss ZEN software to generate binary masks using a dynamic threshold for each channel of interest (Abeta, huIgG, CD68, and tissue area using DAPI). Data analysis was performed using Microsoft Excel and GraphPad Prism 9.

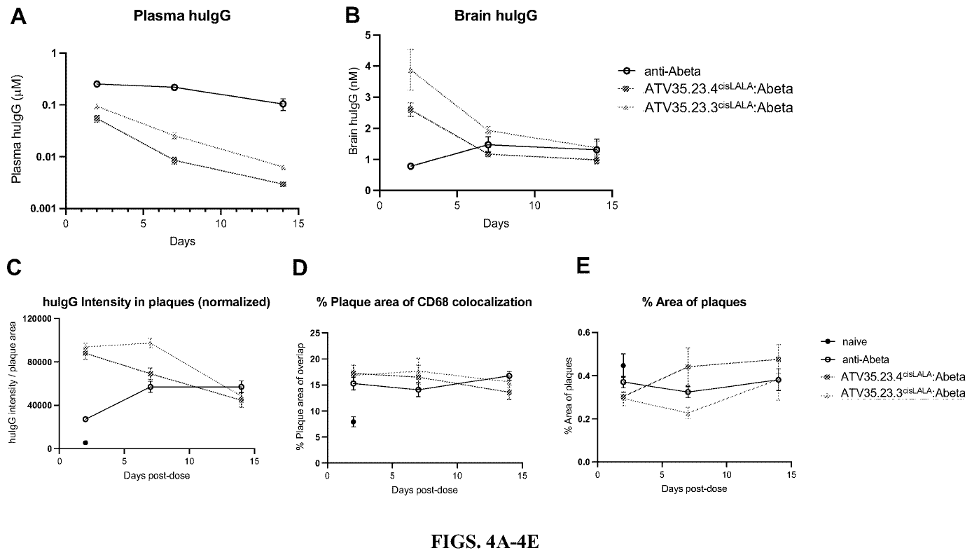

Similar to results in TfR^{mu/hu} KI mice, the plasma PK profile in App^{SAA} KI TfR^{mu/hu} KI mice showed TfR-mediated clearance following a single IV dose of anti-Abeta or ATV:Abeta molecules (FIG. 4A). Brain concentrations at 24 h were approximately 4-5-fold higher for ATVs compared to anti-Abeta (FIG. 4B). Consistent with what was observed in TfR^{mu/hu} KI mice, the ATV35.23.3^{cisLALA}:Abeta resulted in higher brain concentrations compared to the ATV35.23.4^{cisLALA}:Abeta (FIG. 4B). ATVs and anti-Abeta also demonstrated localization of huIgG signal to plaques and immunodecoration correlated well with bulk brain exposure, showing greater levels for ATVs at 48 h post-dose, with a trend toward higher plaque decoration for the weaker affinity ATV:Abeta compared to the stronger affinity ATV:Abeta (FIG. 4C), in agreement with the brain ELISA data but unexpected based on the established TfR affinity-brain concentration relationship. ATVs demonstrated they were effective at recruiting CD68-positive microglia to the plaques by increasing CD68 signal overlap within and surrounding plaques by approximately 2-fold from naïve animals (FIG. 4D). Recruitment of microglial to plaques plays an important role for Abeta therapeutics and enables phagocytosis of plaques. Functionally, this results in a reduction of Abeta plaques from the brain; indeed, ATV35.23.3^{cisLALA}:Abeta showed the greatest reduction in plaques, around 34% reduction 2 days post-dose and 49% reduction one-week post-dose, again consistent with having the highest brain exposure (FIG. 4E).

### Example 5. PK Profile in WT C57BL/6J Mice

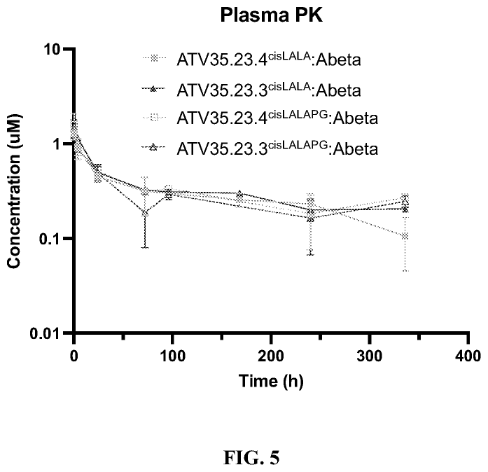

WT C57BL/6J KI mice received a single 10 mg/kg IV dose of ATV:Abeta molecules (ATV35.23.4^{cisLALA}:Abeta ATV35.23.3^{cisLALA}:Abeta, ATV35.23.4^{cisLALAPG}:Abeta, and ATV35.23.3^{cisLALAPG}:Abeta) (n=3/group/time point). In-life or terminal blood was collected at numerous time points to evaluate huIgG concentrations in plasma. Both ATV:Abeta molecules showed similar clearance and PK profiles in WT mice where the molecules lack TfR binding to murine TfR, suggesting these molecules lack non-specific binding in the absence of target binding. cisLALA and cisLALAPG molecules behaved similarly in WT mice (Table 5A. and FIG. 5).

TABLE 5A

| | CL | T1/2 | Cmax/D |
|---|---|---|---|
| Molecule | (mL/day/kg) | (h) | (kg/mL) |
| ATV35.23.4^{cisLALA}:Abeta | 6.5 | 170 | 0.0188 |
| ATV35.23.3^{cisLALA}:Abeta | 4.6 | 189 | 0.0189 |

### Example 6. Ex Vivo Microglia Phagocytosis Reveals Comparable Ability of ATV35.23.3^{cisLALA}:Abeta to Promote Amyloid Beta Phagocytosis Compared to Anti-Abeta

To generate FAM (Fluorescein)-labelled amyloid beta fibrils, FAM-labeled β-Amyloid (1-42) (0.5 mg, Anaspec AS-23525-05) was resuspended in 100 μL DMSO followed by 1 ml PBS dilution to 100 μM. The solution was incubated at 37° C. with shaking for 24 hrs. The FAM-amyloid beta fibrils were then transferred to a 1.5 mL ultracentrifuge tube and centrifuged at 100,000×g for 30 min at 4° C. The supernatant was discarded, and the pellet resuspended in 1 mL PBS followed by extensive pipetting. The ultracentrifugation step was repeated followed by two additional rounds of washing. The pellet was finally resuspended in a volume of 111 μL PBS and stored in the −80° C. freezer.

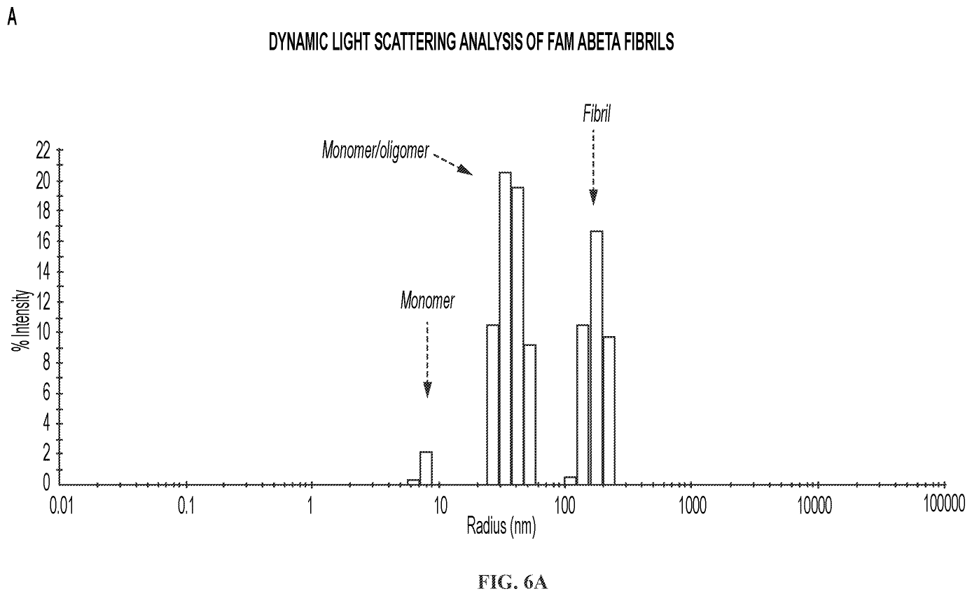

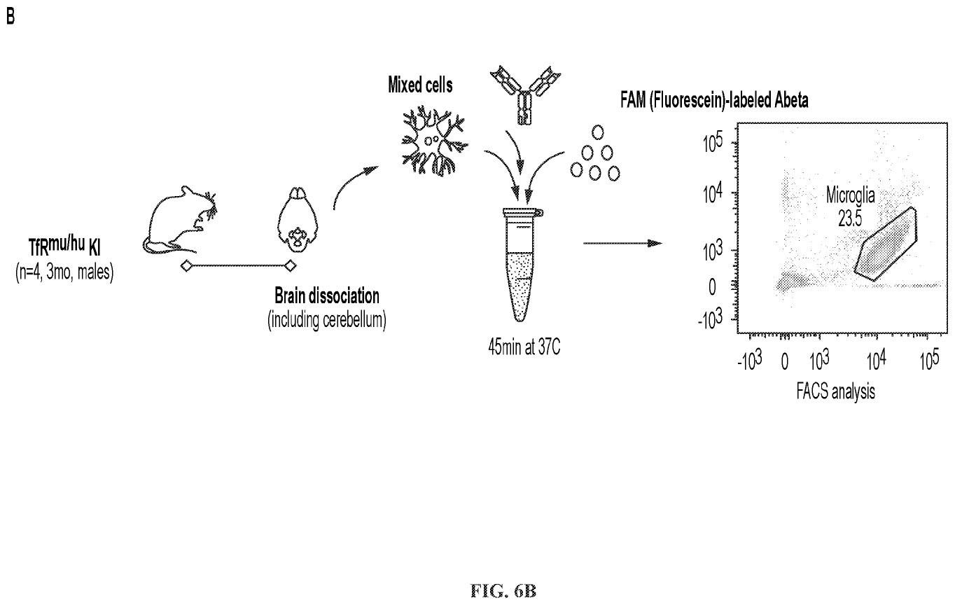

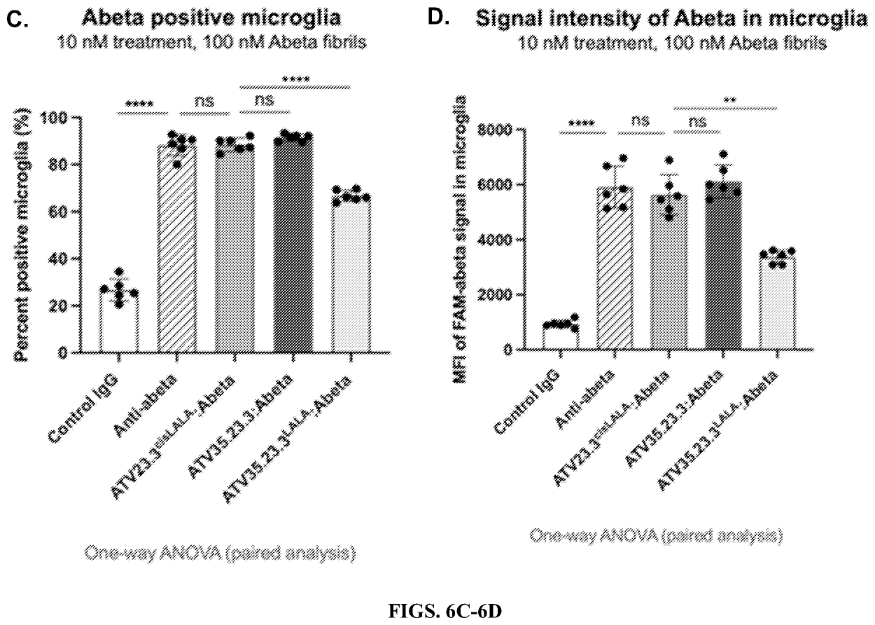

Dynamic Light Scattering (DLS) confirmed the identity of the amyloid beta aggregates. Briefly, 30 μL of amyloid beta aggregates at 1 mg/mL were loaded onto a 384-well plate (black with clear bottom, Costar). The plate cover was used to protect the bottom of the plate from dust and scratches which could affect the DLS reading. The plate was then sealed with a film and centrifuged at 1000 rpm for 5 min to allow the solution to settle to the bottom of the plate and eliminate bubbles. The readings were then performed using the DynaPro Plate Reader III (Wyatt Technology) with the Dynamics v7 software at 25° C. with a 5 second reading per well and an average of 10 measurements. The DLS results revealed that the generated FAM-amyloid beta 1-42 fibrils consist of a heterogenous mixture of monomer, oligomers, and fibrils (FIG. 6A and Tale 6A).

TABLE 6A

| | Radius | Mw-R | | | | |
|---|---|---|---|---|---|---|
| Peak | (nm) | (kDa) | % Pd | % Intensity | % Mass | % Number |
| 1 | 7.8 | 410.9 | 7.69 | 2.5 | 50.6 | 1 × 10² |
| 2 | 38.3 | 17008.9 | 22.71 | 60.0 | 19.1 | 4.8 × 10⁻¹ |
| 3 | 177.0 | 612454.4 | 18.21 | 37.5 | 30.3 | 5.3 × 10⁻³ |

To perform the ex vivo microglia phagocytosis experiment, naïve TfR^{mu/hu} KI (3 months old, males) were perfused with 1×PBS and their brains were used for single cell dissociation. The Adult Brain Dissociation Kit (Miltenyi Biotec, 130-107-677) along with the gentleMACS™ Octo Dissociator was used to dissociate the cells following the kit's instructions. Excess debris and myelin were removed using the kit's Debris Removal Solution, and the final cell pellet was resuspended in 200 μL 0.5% BSA in dPBS (with calcium and magnesium). The number of live microglia per sample were then quantified by staining a small fraction of the cells from each sample with Cd11b-BV421 (BioLegend 101251, 1:100), CD45-APC (BD Biosciences Cat 559864, 1:100), and Fc block (BioLegend 101320, 1:100) for 15 min at 4° C. followed by washes and resuspension in FACS buffer (1% BSA+1 mM EDTA in PBS) containing propidium iodide (PI) (Miltenyi Biotec, 130-093-233). The cell fraction was then mixed with CountBright Plus Absolute Counting Beads (Invitrogen, RefC36995) and loaded on the BD FACSAria III sorter to quantify the total number of live microglia per sample. Cells from the same animals were partitioned into treatment groups such that each group contained 50,000 live microglia treated with 10 nM of IgG control, anti-Abeta, ATV^{cisLALA}:Abeta, ATV:Abeta, or ATV^{LALA}:Abeta along with 100 nM of FAM-labelled amyloid beta fibrils and incubated for 45 min at 37° C. (FIG. 6B). Control samples were treated with 10 nM of the respective antibody and 100 nM FAM-Abeta fibrils but incubated at 4° C. instead for 30 minutes. Following these incubations, the treated samples were washed and stained with Cd11b-BV421 (BioLegend 101251, 1:100), CD45-APC (BD Pharmingen, 1:100), and Fc block (BioLegend 101320, 1:100) for 15 min at 4° C. followed by washes and resuspension in FACS buffer containing PI. On the sorter, 10,000 live microglia were recorded per treated sample along with the intensity of the FAM signal. To quantify the intensity of FAM signal per live microglial cells analysis was performed on FlowJo 10.8.1.

Results revealed a significant increase in the percent of microglia treated with anti-Abeta antibody compared to control IgG (FIG. 6C). Interestingly, there was an equivalent ability of the ATV^{cisLALA}:Abeta to promote amyloid beta phagocytosis compared to both anti-Abeta and ATV:Abeta (FIG. 6C) (One-way ANOVA, paired analysis). In contrast, ATV^{LALA}:Abeta, which has the effector-mitigating LALA mutations on both Fc strands, promoted significantly reduced microglia phagocytosis compared to both ATV^{cisLALA}:Abeta and ATV:Abeta molecules (FIG. 6C) (One-way ANOVA, paired analysis). The mean fluorescence intensity of the FAM-Abeta signal per live microglia suggests the ATV^{cisLALA}:Abeta promotes equivalent level of uptake of amyloid beta into microglia compared to anti-Abeta and ATV:Abeta, whereas the fully effectorless ATV^{LALA}:Abeta had significantly reduced amount of phagocytosed Abeta (FIG. 6D). These results reveal that the cisLALA mutation does not reduce the capacity of the ATV^{cisLALA}:Abeta to trigger microglial phagocytosis.

### Example 7. Single-Dose Immunodecoration and Microglia Recruitment in APP^{SAA} KI; TfR^{mu/Hu} KI Mice Demonstrate Effectiveness of the cisLALA Molecules

APP^{SAA} KI; TfR^{mu/hu} KI mice received a single 3 mg/kg IV dose of control IgG, 3 mg/kg ATV:Abeta, 2 mg/kg ATV^{cisLAA}:Abeta, 3 mg/kg ATV^{LALA}:Abeta, or 7 mg/kg of anti-Abeta (n=8/group). Fresh brain was collected following perfusion at 7 days post-dose. These dose levels achieved approximately equal brain concentrations of anti-Abeta and ATV:Abeta at the terminal time point. Additionally, one perfused, fresh brain hemisphere per animal was immersion fixed for immunohistochemistry and image quantification as described above.

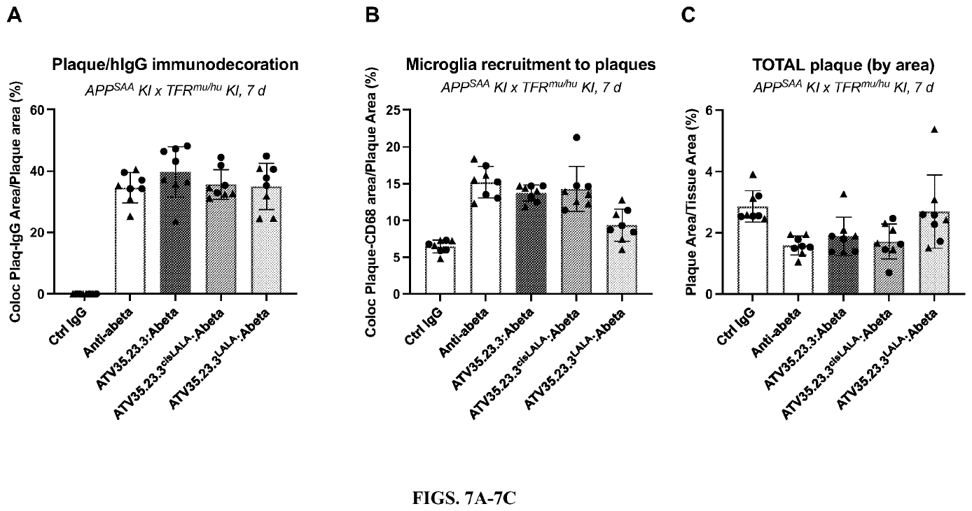

The results revealed as expected an equivalent brain coverage of huIgG per plaque area as shown by the immunodecoration analysis (FIG. 7A). Intriguingly, despite carrying one copy of the LALA mutation the ATV^{cisLALA}:Abeta was able to recruit activated microglia to the plaque similar to both ATV:Abeta and anti-Abeta. Yet, the ATV^{LALA}:Abeta which carries two copies of the LALA mutation revealed reduced capacity (FIG. 7B). The ability of each molecule to recruit microglia to amyloid beta plaques of different sizes (30-125, 125-250, 250-500, and >500 μm²) was also analyzed. The results reveal consistent ability of the ATV^{cisLALA}:Abeta to recruit activated microglia various sizes of amyloid beta plaques, similar to both anti-Abeta and ATV:Abeta. By assessing the total level of plaque area across the groups we observed that ATV^{cisLALA}:Abeta, similar to both anti-Abeta and ATV:Abeta, promoted a significant reduction in plaque levels (FIG. 7C-D). This was also evident when analyzing the results using plaque counts and various sizes of amyloid beta plaques. These results together reveal that the ATV^{cisLALA}:Abeta maintains the ability to recruit microglia to the plaques and to reduce plaques at levels similar to anti-Abeta. In contrast, ATV^{LALA} Abeta is impaired in recruiting microglia and reducing plaques compared to anti-Abeta.

### Example 8. CisLALA Mitigates TFR-Mediated In Vivo Hematology Liability in Non-Human Primates (NHP)

Cynomolgus monkeys received 15 or 50 mg/kg IV doses on days 1, 15, and 29 (e.g., every two weeks) of anti-Abeta or ATV:Abeta molecules. Blood was collected for hematology one week before dosing and on days 8, 15 (prior to day 15 dose administration), 29 (prior to day 29 dose administration), and 31.

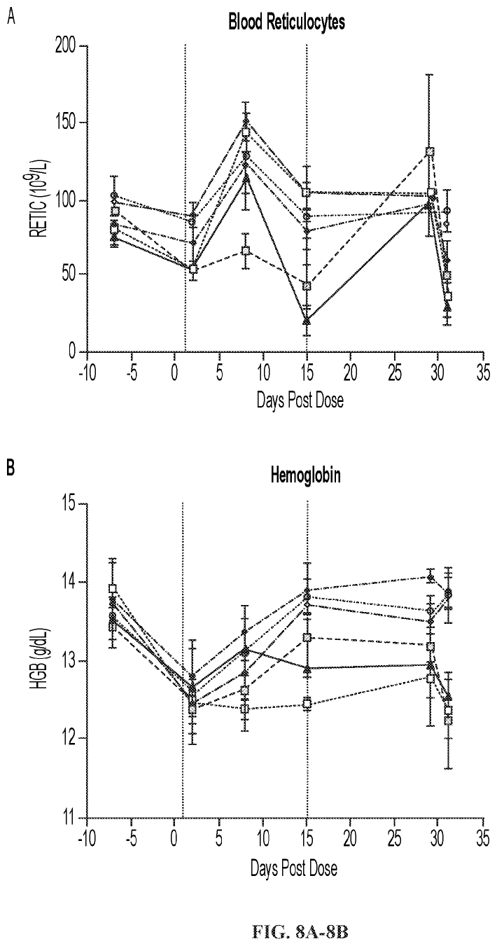

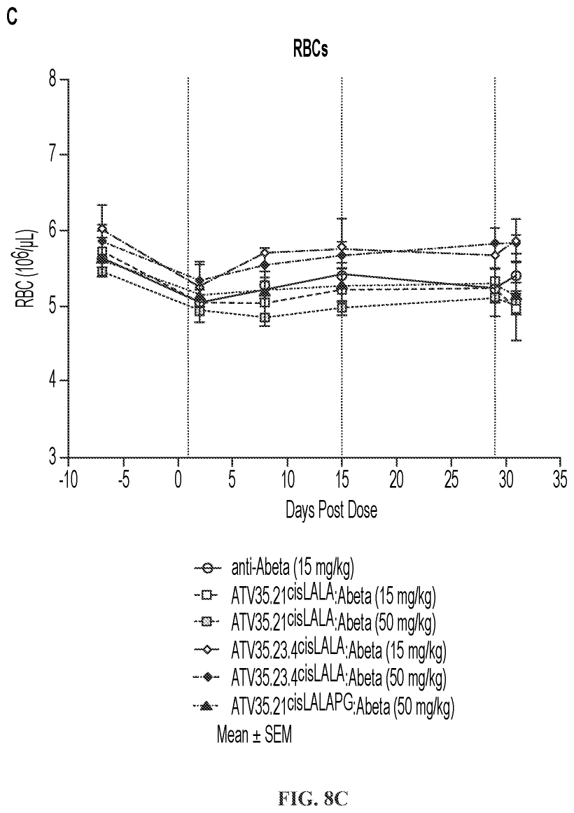

Consistent with previous data, a dose- and TfR affinity-dependent acute reduction in circulating reticulocytes was observed (FIG. 8A). There was minimal impact to circulating RBCs and hemoglobin after 4 weeks of exposure and there were no differences observed between cisLALA and cisLALAPG ATV:Abeta molecules (FIG. 8B-C). These data suggests that multi-dosing of ATVs has a favorable safety profile in NHP.

### Example 9. PK of Single Dose ATV:Abeta in Cynomolgus Monkeys

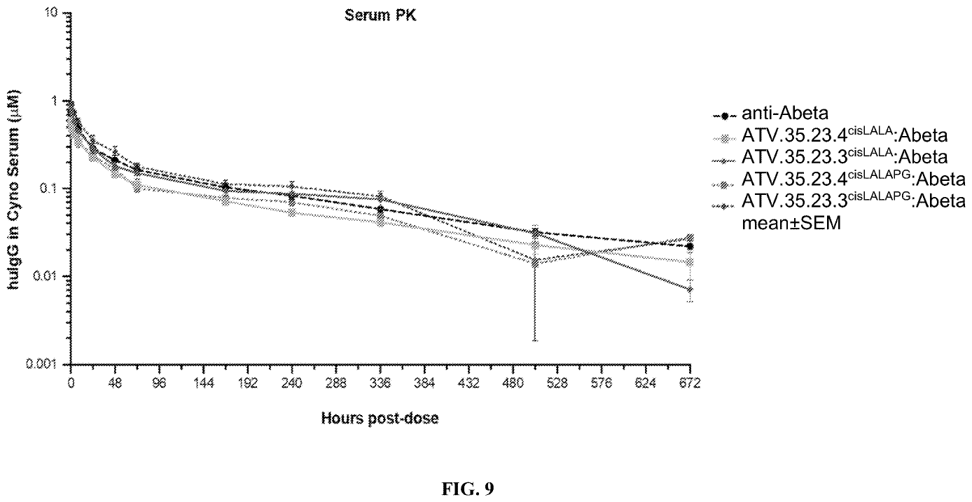

Cynomolgus monkeys received a single 6 mg/kg IV dose of anti-Abeta or ATV:Abeta molecules. Blood was collected at various time points for measurement of huIgG concentrations. The area under the curve (AUC) and clearance of ATV35.23.3^{cisLALA}:Abeta and ATV35.23.3^{cisLALAPG}:Abeta was similar to the anti-Abeta control group in non-human primates (FIG. 9). The clearance of ATV35.23.4^{cisLALA}:Abeta and ATV35.23.4^{cisLALAPG}:Abeta slightly faster than anti-Abeta.

### Example 10. PK Profile and Plaque Microglia Recruitment of ATV with cisLALA or cisLALAPG Mutations in 5XFAD; TfR^{mu/Hu} KI Mice

5XFAD; TfR^{mu/hu} KI mice received four IP doses (q3d, i.e., days 0,3,6,9) of ATV35.23.4^{cisLALA}, ATV35.23.4^{cisLALAPG}, or control IgG (n=15/group). In-life plasma was collected from n=3 mice per group per time point and from all animals terminal blood and fresh brain (following perfusion) were collected and snap frozen at 12 days post-dose to evaluate huIgG concentrations in plasma and brain lysate. One fresh brain hemisphere per animal was immersion fixed for approximately 24 h at 4° C. for immunohistochemistry. Three to four replicate sagittal brain sections (40 μm) were selected for each animal and stained by incubation in blocking buffer, incubation overnight in primary/secondary antibodies (CD68, Biorad, MCA1957; Abeta, IBL America 18584; and donkey-anti-huIgG, Jackson, 709-606-149) at 4° C., followed by washes and incubation with secondary antibodies (donkey-anti-rat, Invitrogen, SA5-10027; donkey-anti-rabbit, Invitrogen, A21206) and DAPI (5 μg/mL, Invitrogen, D1306), before washes and coverslipping with Prolong Glass (Invitrogen, P36984). Slides were imaged using a Zeiss Axioscan.Z1 slide scanner at 20× magnification and processed using custom macros in Zeiss ZEN software to generate binary masks using a dynamic threshold for each channel of interest (Abeta, huIgG, CD68, and tissue area using DAPI). Data analysis was performed using Microsoft Excel and GraphPad Prism 9.

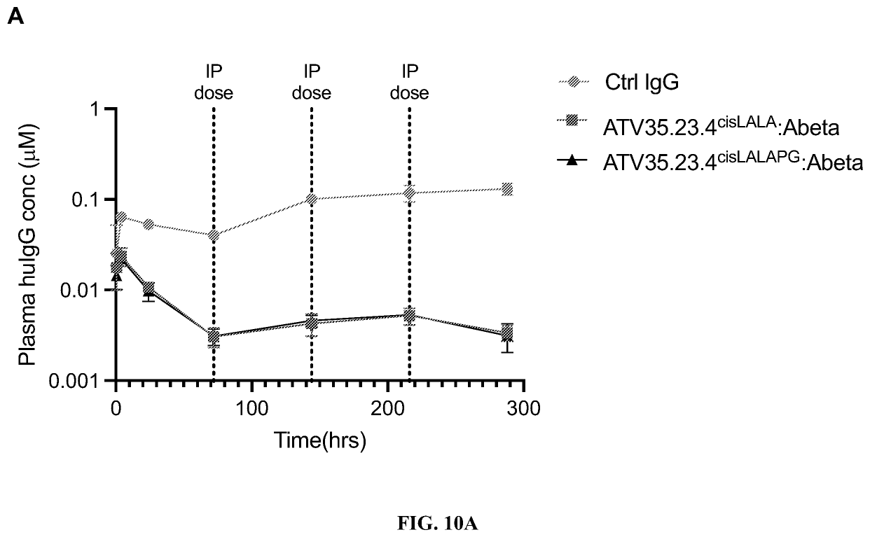

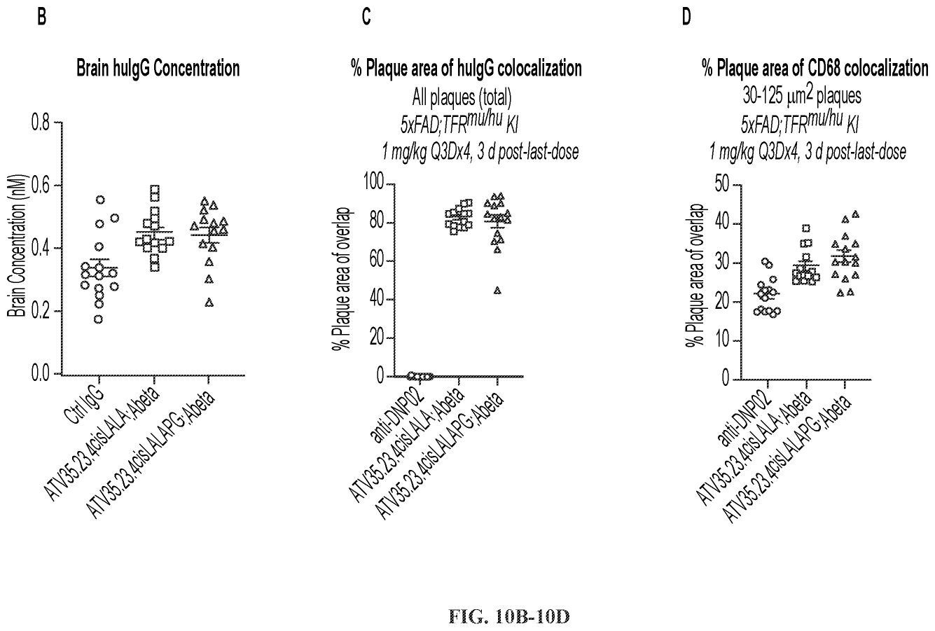

The plasma PK profiles in the 5XFAD; TfR^{mu/hu} KI mice were identical between ATV35.23.4^{cisLALA} and ATV35.23.4^{cisLALAPG} and showed expected TfR-mediated clearance versus control IgG (FIG. 10A). Brain concentrations of the ATVs were also similar to one another (FIG. 10B), indicating that the different cisLALAPG mutations did not overall impact clearance from plasma or uptake into the brain compared to cisLALA mutations.

This relatively low dose used aimed to avoid hitting a ceiling effect; this would enable detection of small differences in microglial recruitment (indicating Fc-mediated response) between Fc mutations on the ATVs. This endpoint focused on the smallest plaques given the acute nature of the study, as the time was expected to be insufficient for substantial response around larger plaques. Immunodecoration of all plaques by both ATVs was equivalent (FIG. 10C), in agreement with the bulk brain huIgG concentrations. Interestingly, ATV35.23.4^{cisLALAPG} showed equivalent ability to recruit microglia to small plaques as ATV35.23.4^{cisLALA} (FIG. 10D) indicating that the additional effector function silencing mutation did not impact microglial recruitment. This suggests that the asymmetrical engineering still allows the molecule to retain essentially full Fab-mediated effector function.

### Example 11. ARIA Safety Study in 5XFAD; TfR^{mu/Hu} KI Mice

5xFAD: TfRmu/huKI mice (male and female mice, 10-12 months of age at the start of the study) were allocated to 5 treatment groups (n=10): anti-Abeta (10 mg/kg, n=6M, 4F), anti-Abeta with LALA mutations (10 mg/kg, n=6M, 4F), ATV35.23.3:Abeta (3 mg/kg, n=6M, 3F), ATV35.23.3cisLALA:Abeta (3 mg/kg, n=4M, 4F) and naïve (n=3M, 2F).

All treatment were administered i.p. weekly for 10 weeks, and MRI imaging was first collected at baseline, prior to initiation of treatment, and performed within 1-3 days after weekly dosing. Blood was collected from all animals prior to the first dose. Naïve animals were imaged at baseline and after dose 10 only. Anti-CD4 antibody was administered i.p. every 2 weeks, starting 1 day before the first dosing of compounds (0.5 mg/animal). At the end of the study, blood was collected prior to the 10th and last dose, and at sacrifice 24 h after the last dose.

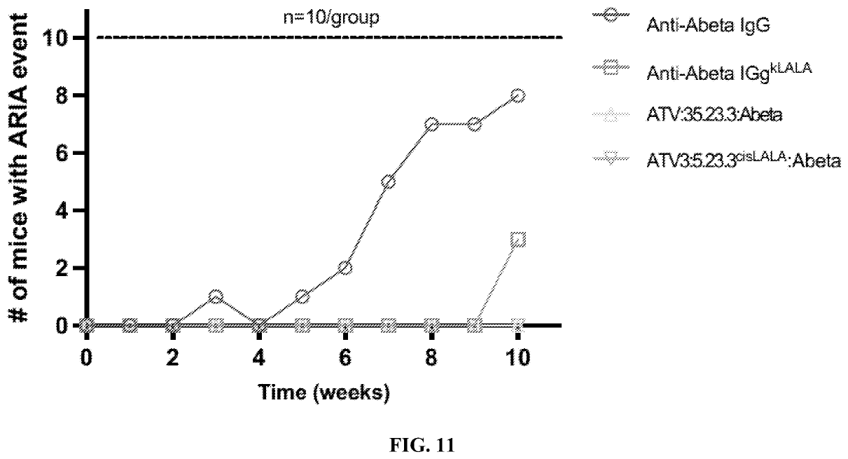

Analysis of the weekly MRI scans showed occurrence of MRI lesions consistent with ARIA-E and ARIA-H in 8/10 animals in the anti-Abeta group, and 3/10 animals in the Abeta with LALA mutations group. No ARIA could be detected in any of the 2 ATV treatment groups (FIG. 11).

MRI lesions detected with the T2w sequence were either located to the meninges surrounding the brain (convexity hyperdensity signal), or within the depth of the cortex (cortical hyperdensity signal, either diffuse or focal). MRI lesions detected with the T2* sequence appeared as focal hypodense signals in the meninges or the cortex. The underlying pathology was characterized by meningovascular inflammation and microvascular lesions (visualized using H&E staining), leakage of proteinaceous fluids (visualized by immunohistochemistry for albumin and/or mouse IgG), and presence of microhemorrhages (visualized with Perls staining).

Analysis of the MRI scans and histological sections were done independently, and by different observers who were blinded for the treatment allocation.

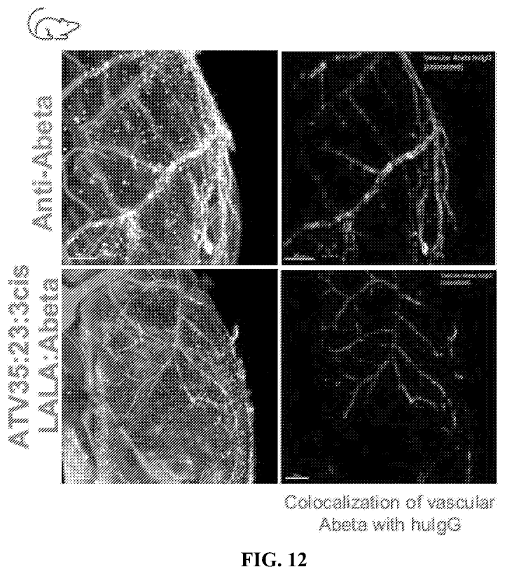

A separate experiment was performed to evaluate the route of entry into the brain of ATV-Abeta. As shown in FIG. 12, mice administered with a single dose of ATV35.23.3cisLALA:Abeta display less vascular binding, as compared to mice administered with a single dose of the anti-Abeta treatment. These results demonstrate a different route of entry into the brain, that may result in lower incidence of ARIA events.

1. An Abeta-targeting protein comprising:
(a) an amyloid beta (Abeta)-binding region;
(b) a transferrin receptor (TfR)-binding region that specifically binds a TfR with an affinity of about 900 nM to about 10,000 nM; and
(c) an Fcγ Receptor (FcγR)-binding region.

2. The Abeta-targeting protein of claim 1, wherein the TfR-binding region specifically binds TfR with an affinity of about 900 nM to about 2500 nM.

3. The Abeta-targeting protein of claim 1, wherein the TfR-binding region specifically binds TfR with an affinity of about 900 nM to about 1300 nM.

4. The Abeta-targeting protein of claim 3, wherein the TfR-binding region specifically binds TfR with an affinity of about 1100 nM.

5. The Abeta-targeting protein of any one of claims 1-4, wherein the TfR-binding region binds to an apical domain of the TfR.

6. The Abeta-targeting protein of any one of claims 1-5, wherein TfR-binding region binds the TfR without inhibiting binding of transferrin to the TfR.

7. The Abeta-targeting protein of any one of claims 1-6, wherein the Abeta-targeting protein is capable of being actively transported across the blood brain barrier.

8. The Abeta-targeting protein of any one of claims 1-7, wherein the TfR-binding region comprises:
(a) a TfR-binding polypeptide;
(b) a TfR-binding polypeptide linked to an Fc polypeptide;
(c) a first Fc polypeptide comprising a CH3 domain modified to bind to TfR;
(d) an anti-TfR antibody or antigen-binding fragment thereof; or
(e) an anti-TfR antibody or antigen-binding fragment thereof linked to an Fc polypeptide.

9. The Abeta-targeting protein of any one of claims 1-8, wherein the FcγR-binding region comprises a second Fc polypeptide, optionally where the second Fc polypeptide does not contain any modifications that reduce FcγR binding.

10. The Abeta-targeting protein of claim 9, wherein the Abeta-targeting protein comprises an Fc dimer.

11. The Abeta-targeting protein of claim 10, wherein the Fc dimer comprises:
(a) the first Fc polypeptide; and
(b) the second Fc polypeptide.

12. The Abeta-targeting protein of any one of claims 8-11, wherein the first Fc polypeptide, the second Fc polypeptide, or the first Fc polypeptide and the second Fc polypeptide are derived from a human IgG1, IgG2, IgG3, or IgG4.

13. The Abeta-targeting protein of any one of claims 8-12, wherein the modified CH3 domain comprises amino acid substitutions 384Y, 386T, 387E, 388W, 389V, 413T, 415E, 416E, and 421F, according to EU numbering scheme.

14. The Abeta-targeting protein of any one of claims 8-10 and 12-13, wherein anti-TfR antibody or antigen-binding fragment thereof comprises: an antibody, a F(ab)2 fragment, a Fab fragment, or a single chain variable fragment (scFv).

15. The Abeta-targeting protein of any one of claims 1-14, wherein the Abeta-targeting protein has reduced binding to an FcγR when the Abeta-targeting protein is bound to the TfR.

16. The Abeta-targeting protein of any one of claims 9-15, wherein the Fc dimer comprises the first Fc polypeptide, wherein the first Fc polypeptide comprises one or more amino acid substitutions that reduce binding to an FcγR, and the second Fc polypeptide, wherein the second Fc polypeptide does not contain any modifications that reduce FcγR binding.

17. The Abeta-targeting protein of claim 16, wherein the one or more amino acid substitutions that reduce binding to the FcγR comprise 234A and 235A substitutions, according to EU numbering scheme.

18. The Abeta-targeting protein of claim 16 or 17, wherein the one or more amino acid substitutions that reduce binding to the FcγR comprises a 329G substitution, according to EU numbering scheme.

19. The Abeta-targeting protein of claim 16, wherein the one or more amino acid substitutions that reduce binding to the FcγR comprise 234A, 235A, and 329G substitutions, according to EU numbering scheme.

20. The Abeta-targeting protein of any one of claims 10-19, wherein the Fc dimer comprises one or more heterodimerizing mutations.

21. The Abeta-targeting protein of claim 20, wherein the first Fc polypeptide comprises a knob mutation and the second Fc polypeptide comprises a hole mutation, or wherein the first Fc polypeptide comprises a hole mutation and the second Fc polypeptide comprises a knob mutation.

22. The Abeta-targeting protein of claim 21, wherein the knob mutation comprises a T366W substitution, according to EU numbering scheme; and the hole mutation comprises T366S, T368A, and Y407V substitutions, according to EU numbering scheme.

23. The Abeta-targeting protein of claim 22, wherein the first Fc polypeptide comprises T366S, T368A, and Y407V substitutions, according to EU numbering scheme; and the second Fc polypeptide comprises a T366W substitution, according to EU numbering scheme.

24. The Abeta-targeting protein of claim 22, wherein the first Fc polypeptide comprises a T366W substitution, according to EU numbering scheme; and the second Fc polypeptide comprises T366S, T368A, and Y407V substitutions, according to EU numbering scheme.

25. The Abeta-targeting protein of claim 24, wherein the modified CH3 domain of the first Fc polypeptide has at least 85% identity, at least 90% identity, or at least 95% identity to amino acids 348-454 of any of SEQ ID NOs:8, 10, and 17-31.

26. The Abeta-targeting protein of claim 25, wherein the modified CH3 domain comprises the amino acid sequence of amino acids 348-453 or 348-454 of any of SEQ ID NOs:8, 10, and 17-31.

27. The Abeta-targeting protein of claim 24-26, wherein the first Fc polypeptide has at least 85% identity, at least 90% identity, or at least 95% identity to amino acids 228-454 of any of SEQ ID NOs:8, 10, and 17-31.

28. The Abeta-targeting protein of claim 27, wherein the first Fc polypeptide comprises the amino acid sequence of amino acids 228-453 or 228-454 of any of SEQ ID NOs: 8, 10, and 17-31.

29. The Abeta-targeting protein of any one of claims 24-27, wherein the second Fc polypeptide comprises least 85% identity, at least 90% identity, or at least 95% identity to amino acids 228-454 of SEQ ID NO:9.

30. The Abeta-targeting protein of claim 29, wherein the second Fc polypeptide comprises the amino acid sequence of amino acids 228-453 or 228-454 of SEQ ID NO:9.

31. The Abeta-targeting protein of any one of claims 1-30, wherein the Abeta-binding region comprises an anti-Abeta antibody or an antigen binding fragment thereof.

32. The Abeta-targeting protein of claim 31, wherein the anti-Abeta antibody or an antigen binding fragment thereof comprises: at least one Fab region, at least two Fab regions, an F(ab)2 region, or at least one scFv.

33. The Abeta-targeting protein of claim 31 or 32, wherein the anti-Abeta antibody or an antigen binding fragment thereof comprises a heavy chain variable region (VH) and a light chain variable region (VL), wherein:
(a) the VH comprises a first heavy chain complementarity determining region (VHCDR1) having the amino acid sequence of SEQ ID NO:1, a second heavy chain complementarity determining region (VHCDR2) having the amino acid sequence of SEQ ID NO: 2, and a third heavy chain complementarity determining region (VHCDR3) having the amino acid sequence of SEQ ID NO:3; and
(b) the VL comprises a first light chain complementarity determining region (VLCDR1) having the amino acid sequence of SEQ ID NO:4, a second light chain complementarity determining region (VLCDR2) having the amino acid sequence of SEQ ID NO: 5, and a third light chain complementarity determining region (VLCDR3) having the amino acid sequence of SEQ ID NO:6.

34. The Abeta-targeting protein of claim 33, wherein the VH comprises an amino acid sequence having at least 85% identity, at least 90% identity, or at least 95% identity to amino acids 1-120 of SEQ ID NO:8 or 10 and the VL comprises an amino acid sequence having at least 85% identity, at least 90% identity, or at least 95% identity to amino acids 1-109 of SEQ ID NO:7.

35. The Abeta-targeting protein of claim 34, wherein the VH comprises the amino acid sequence of amino acids 1-120 of SEQ ID NO:8 or 10 and the VL the amino acid of amino acids 1-109 of SEQ ID NO:7.

36. The Abeta-targeting protein of claim 33, wherein the anti-Abeta antibody or an antigen binding fragment thereof comprises two heavy chain Fab regions and two light chain Fab regions, wherein each heavy chain Fab region comprises an amino acid sequence having at least 85% identity, at least 90% identity, or at least 95% identity to amino acids 1-227 of SEQ ID NO:8 or 10 and each light chain Fab regiwon comprises an amino acid sequence having at least 85% identity, at least 90% identity, or at least 95% identity to SEQ ID NO: 7.

37. The Abeta-targeting protein of claim 36, each heavy chain Fab region comprises the amino acid sequence of amino acids of 1-227 of SEQ ID NO:8 or 10 and each light chain Fab region comprises the amino acid sequence of SEQ ID NO:7.

38. The Abeta-targeting protein of any one of claims 1-37, wherein the Abeta-targeting protein comprises:
(a) a first light chain comprising an amino acid sequence having at least 85% identity, at least 90% identity, or at least 95% identity to SEQ ID NO:7;
(b) a first heavy chain comprising an amino acid sequence having at least 85% identity, at least 90% identity, or at least 95% identity to SEQ ID NO:8 or 10;
(c) a second light chain comprising an amino acid sequence having at least 85% identity, at least 90% identity, or at least 95% identity to SEQ ID NO:7; and
(d) a second heavy chain comprising an amino acid sequence having at least 85% identity, at least 90% identity, or at least 95% identity to SEQ ID NO:9.

39. The Abeta-targeting protein of claim 38, wherein:
(a) the first light chain comprises the amino acid sequence of SEQ ID NO: 7;
(b) the first heavy chain comprises the amino acid sequence of SEQ ID NO: 8 or 10, optionally wherein the C terminal lysine is removed;
(c) the second light chain comprised the amino acid sequence of SEQ ID NO: 7; and
(d) the second heavy chain
comprises the amino acid sequence of SEQ ID NO:9, optionally wherein the C terminal lysine is removed.

40. The Abeta-targeting protein of any one of claims 1-39, wherein the Abeta-targeting protein binds to amyloid plaques, cerebrovascular Abeta, or diffuse Abeta deposits.

41. The Abeta-targeting protein of one of claims 1-40, wherein the Abeta-targeting protein does not deplete reticulocytes in vivo.

42. A composition comprising the Abeta-targeting protein of any one of claims 1-41.

43. A pharmaceutical composition comprising the Abeta-targeting protein of any one of claims 1-41 or the composition of claim 42, and a pharmaceutically acceptable excipient.

44. The pharmaceutical composition of claim 43, wherein the pharmaceutical composition further comprises an additional agent useful for treating Alzheimer's disease.

45. The pharmaceutical composition of claim 43, wherein the pharmaceutical composition is formulated for use in combination with an additional agent useful for treating Alzheimer's disease.

46. A method of reducing amyloid plaques in a brain of a subject, the method comprising administering to the subject the Abeta-targeting protein of any one of claims 1-41, or the composition of claim 42 or the pharmaceutical composition of any one of claims 43-45.

47. The method of claim 46, wherein the subject has cognitive impairment, memory loss, dementia, or loss of neuronal connections in the brain.

48. The method of claim 46 or 47, wherein the subject has or is a risk of developing a disease associated with accumulation of amyloid plaques.

49. The method of any one of claims 46-48, wherein the subject has or has been diagnosed with Alzheimer's disease or is at increased risk of developing Alzheimer's disease.

50. A method of treating a neurodegenerative disease in a subject, comprising administering to the subject the Abeta-targeting protein of any one of claims 1-41, or the composition of claim 42 or the pharmaceutical composition of any one of claims 43-45.

51. The method of claim 50, wherein the neurodegenerative disease is Alzheimer's disease.

52. The method of any one of claims 46-51, wherein the method further comprises administering to the subject at least one additional agent useful for treating Alzheimer's disease.

53. The pharmaceutical composition of any one of claims 43-45 for use in the treatment of Alzheimer's disease.

54. The pharmaceutical composition of any one of claims 43-45 for use in increasing Abeta phagocytosis and/or increasing recruitment of microglia to Abeta-positive plaques in a subject.

55. A nucleic acid encoding the Abeta-targeting protein of any one of claims 1-41.

56. A nucleic acid encoding a polypeptide having at least 85% identity, at least 90% identity, or at least 95% identity, to any of SEQ ID NOs: 8, 10, and 17-31, wherein the encoded polypeptide has the amino acids at positions 384, 386, 387, 388, 389, 390, 413, 415, 416, and 421, according to EU numbering scheme, as shown in Table 1.

57. The nucleic acid of claim 56, wherein the encoded polypeptide contains:
(a) an alanine at position 234 and an alanine at position 235;
(b) a glycine at position 329;
(c) an alanine at position 234, an alanine at position 235, and a glycine at position 329;
(d) a tryptophan at position 366;
(e) a serine at position 366, an alanine at position 368, and a valine at position 407.
(f) an alanine at position 234, an alanine at position 235, and a tryptophan at position 366;
(g) a glycine at position 329 and a tryptophan at position 366;
(h) an alanine at position 234, an alanine at position 235, a glycine at position 329, and a tryptophan at position 366;
(i) an alanine at position 234, an alanine at position 235, a serine at position 366, an alanine at position 368, and a valine at position 407;
(j) a glycine at position 329, a serine at position 366, an alanine at position 368, and a valine at position 407; or
(k) an alanine at position 234, an alanine at position 235, a glycine at position 329, a serine at position 366, an alanine at position 368, and a valine at position 407,
wherein each position is according to EU numbering scheme.

58. The nucleic acid of claim 57, wherein the encoded polypeptide has the amino acid sequence of any of SEQ ID NOs: 8, 10, and 17-31, optionally wherein the polypeptide has a glycine at position 329 and/or a tryptophan at position 366 according to EU numbering scheme.

59. A combination of nucleic acids comprising: a first nucleic acid encoding a first polypeptide having the amino acid sequence of any of SEQ ID NOs: 8, 10, and 17-31 and a second nucleic acid encoding a second polypeptide having the amino acid sequence of SEQ ID NO: 9.

60. The combination of claim 59, wherein the combination further comprises a third nucleic acid encoding a third polypeptide having the amino acid sequence of SEQ ID NO: 7.

61. A method of producing an Abeta-targeting protein comprising:
(a) culturing a recombinant host cell comprising one or more nucleic acids encoding the Abeta-targeting protein of any one of claims 1-41; and
(b) isolating the Abeta-targeting protein from the culture.

62. A cell expressing the Abeta-targeting protein of any one of claims 1-41.

63. The cell of claim 62, wherein the cell comprises:
(a) a first nucleic acid encoding a first polypeptide having at least 85% identity, at least 90% identity, at least 95% identity, or 100% identify to any of SEQ ID NOs: 8, 10, and 17-31, wherein the encoded polypeptide has the amino acids at positions 384, 386, 387, 388, 389, 390, 413, 415, 416, and 421, according to EU numbering scheme, as shown in Table 1;
(b) a second nucleic acid encoding a second polypeptide having at least 85% identity, at least 90% identity, at least 95% identity, or 100% identify to SEQ ID NO:9; and
(c) a third nucleic acid encoding a third polypeptide having at least 85% identity, at least 90% identity, at least 95% identity, or 100% identify to SEQ ID NO:7.

64. The cell of claim 63, wherein the first polypeptide comprises:
(a) an alanine at position 234 and an alanine at position 235;
(b) a glycine at position 329;
(c) an alanine at position 234, an alanine at position 235, and a glycine at position 329;
(d) a tryptophan at position 366;
(e) an alanine at position 234, an alanine at position 235, and a tryptophan at position 366;
(f) a glycine at position 329 and a tryptophan at position 366;
(g) an alanine at position 234, an alanine at position 235, a glycine at position 329, and a tryptophan at position 366;
wherein each position is according to EU numbering scheme.


SEQUENCE LISTING (31 sequences)

SEQ ID NO: 1 - protein, 5 aa, synthetic construct
SYGMH

SEQ ID NO: 2 - protein, 17 aa, synthetic construct
VIWFDGTKKYYTDSVKG

SEQ ID NO: 3 - protein, 15 aa, synthetic construct
DRGIGARRGPYYMDV

SEQ ID NO: 4 - protein, 11 aa, synthetic construct
RASQSISSYLN

SEQ ID NO: 5 - protein, 7 aa, synthetic construct
AASSLQS

SEQ ID NO: 6 - protein, 9 aa, synthetic construct
QQSYSTPLT

SEQ ID NO: 7 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 9 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWVNYKTTPPVLDSDGSFFLYSKLTVTKEEWQQGFVFSCSVMHEALHNHYTQKSLSLSPGK

SEQ ID NO: 11-14 - skipped (4 entries)

SEQ ID NO: 15 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 16 - protein, 181 aa, Homo sapiens
NSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVS

SEQ ID NO: 17 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGNEWSSYKTTPPVLDSDGSFFLYSKLTVTKSEWQQGFVFSCSVMHEALHNHYTQKSLSLSPGK

SEQ ID NO: 18 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWTSYKTTPPVLDSDGSFFLYSKLTVTKSEWQQGFVFSCSVMHEALHNHYTQKSLSLSPGK

SEQ ID NO: 19 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSSYKTTPPVLDSDGSFFLYSKLTVTKSEWQQGYVFSCSVMHEALHNHYTQKSLSLSPGK

SEQ ID NO: 20 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESFGTEWSSYKTTPPVLDSDGSFFLYSKLTVSKSEWQQGFVFSCSVMHEALHNHYTQKSLSLSPGK

SEQ ID NO: 21 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSSYKTTPPVLDSDGSFFLYSKLTVSKSEWQQGYVFSCSVMHEALHNHYTQKSLSLSPGK

SEQ ID NO: 22 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWTNYKTTPPVLDSDGSFFLYSKLTVTKSEWQQGFVFSCSVMHEALHNHYTQKSLSLSPGK

SEQ ID NO: 23 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWVNYKTTPPVLDSDGSFFLYSKLTVTKSEWQQGFVFSCSVMHEALHNHYTQKSLSLSPGK

SEQ ID NO: 24 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVTKSEWQQGYVFSCSVMHEALHNHYTQKSLSLSPGK

SEQ ID NO: 25 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESFGTEWSNYKTTPPVLDSDGSFFLYSKLTVSKSEWQQGFVFSCSVMHEALHNHYTQKSLSLSPGK

SEQ ID NO: 26 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKSEWQQGYVFSCSVMHEALHNHYTQKSLSLSPGK

SEQ ID NO: 27 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESFGTEWSNYKTTPPVLDSDGSFFLYSKLTVSKSEWQQGYVFSCSVMHEALHNHYTHKSLSLSPGK

SEQ ID NO: 28 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWVNYKTTPPVLDSDGSFFLYSKLTVSKSEWQQGFVFSCSVMHEALHNHYTQKSLSLSPGK

SEQ ID NO: 29 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGNEWSNYKTTPPVLDSDGSFFLYSKLTVSKEEWQQGFVFSCSVMHEALHNHYTQKSLSLSPGK

SEQ ID NO: 30 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWTNYKTTPPVLDSDGSFFLYSKLTVSKEEWQQGFVFSCSVMHEALHNHYTQKSLSLSPGK

SEQ ID NO: 31 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESYGTEWSNYKTTPPVLDSDGSFFLYSKLTVSKEEWQQGYVFSCSVMHEALHNHYTQKSLSLSPGK